(19) United States
(12) Patent Application Publication
(10) Pub. No.: US 2005/0053970 A1
(43) Pub. Date: Mar. 10, 2005

(54) Methods and compositions for identifying peptide aptamers capable of altering a cell phenotype

(76) Inventors: John D. Benson, West Roxbury, MA (US); Sylvie Magali Vincent, Somerville, MA (US); Bradley Bryan Brasher, Natick, MA (US); Zhenwei Miao, San Diego, CA (US); Dudley Lamming, Cambridge, MA (US)

Correspondence Address: ELMORE CRAIG, P.C., 209 MAIN STREET, N. CHELMSFORD, MA, 01863, US

(21) Appl. No.: 10/833,951

(22) Filed: Apr. 28, 2004

Related U.S. Application Data
(63) Continuation of application No. PCT/US02/35584, filed on Nov. 6, 2002.
(60) Provisional application No. 60/357,278, filed on Feb. 14, 2002.
(60) Provisional application No. 60/333,262, filed on Nov. 6, 2001.

(51) Int. Cl. C12Q 1/68; A61K 48/00

(52) U.S. Cl. 435/6; 514/44

(57) ABSTRACT

The invention provides methods and compositions for screening and identifying of peptide aptamers that can modulate a cell phenotype and further, can be used for the treatment of a disease involving a misregulated cell phenotype, such as, for example, a cancer. The invention encompasses methods and compositions for producing cyclic peptide aptamers, including peptide aptamers comprising a conotoxin sequence, having improved stability and bioactivity. The invention also provides methods and compositions for improved gene delivery and expression of a peptide aptamer in cell.

### CROSS-REFERENCE TO RELATED PATENT APPLICATIONS

This application is a continuation of PCT patent application number PCT/US02/35584 (filed Nov. 6, 2002), which claims priority to U.S. provisional patent application Ser. Nos. 60/357,278 (filed Feb. 14, 2002) and 60/333,262 (filed Nov. 6, 2001), all of which are herein incorporated by reference in their entirety.

### RELATED INFORMATION

The contents of all patents, patent applications, and publications cited throughout this specification are hereby incorporated by reference in their entireties.

### BACKGROUND OF THE INVENTION

Peptides are often effective starting points for the design of therapeutic molecules. Peptides can themselves be highly effective therapeutic agents, despite the often attendant issues of delivery, bioavailability, or stability, when they serve as ligands for, e.g., membrane-associated cellular receptors (i.e. agonists or antagonists). In the case of intracellular targets for peptide ligands, in vitro assay methods (e.g. biochemical assays) are most often used to identify effective peptides that manifest desirable activities or effects. Strategies for designing peptides in this latter class include the use of a peptide sequence composing the interaction domain within one partner of a known and important protein-protein interaction to compete and inhibit this interaction in assays where both protein interaction partners are provided and their interaction can be measured quantitatively.

An approach for the discovery of novel peptide ligands that modulate the activity of a known protein target involves the survey of peptide libraries to identify tightly binding peptides. Such libraries typically exist either within a phage display format or are chemically synthesized as combinatorial libraries of random peptides. This approach may be used to identify peptides that bind an enzyme in a manner that affects (increases or decreases) its catalytic properties, or may be used to identify high-affinity peptide ligands that affect interaction of the target protein with other proteins.

In each case, the identification of a peptide with desirable biological activities is only the beginning of a long, and sometimes, impossible process of converting that peptide into a molecule with therapeutic utility. Difficulties in this regard may arise from a number of issues: (i) mammalian cells are not permeable to most peptides, rendering them largely ineffective against intracellular targets; (ii) peptides (especially linear peptides) are most often unstable; (iii) linear peptides are structurally unconstrained, allowing essentially free rotation at each intramolecular bond along the peptide backbone; and, (iv) binding affinities of peptide ligands for their target proteins may be relatively low, often no better than μM or even mM. Moreover, in consideration of these significant shortcomings associated with many peptides, much effort has been devoted to the use of linear peptides as chemical entities to serve as effective conceptual starting points for the design of non-peptidic small molecules. This approach, often referred to as “peptidomimetics” has met with very limited success.

Another type of issue that impacts the ability to identify therapeutic peptides, is the difficulty of specifying a priori a protein target within the cell that would result in a specific desired effect. This dilemma is exemplified by the fact that signal transduction pathways within eukaryotic cells are highly complex, convoluted, interconnected, and redundant. Thus, although it is common to formulate hypotheses regarding the utility of targeting a specific protein for the identification of peptides or other small molecules that might modulate its activity, it is quite common to discover either that it is difficult to direct the activity of such a molecule to that specific target, or that the hypothesized target is irrelevant, superfluous, or redundant with respect to the desired effect or function. In this regard, binding-mediated approaches such as phage display or screening of chemically synthesized peptide libraries for interactors may lead to peptides or peptide leads that do not carry out the desired activity within the cell, or that do so with insufficient potency or specificity.

Accordingly, improved methods and compositions for identifying therapeutic peptides capable, e.g., of altering a cell phenotype, are needed.

### SUMMARY OF THE INVENTION

The present invention provides improved methods and compositions for screening and identifying peptides (aptamers) from random libraries that manifest identifiable phenotypes in cells. The methods and compositions of the invention have several important advantages over prior peptide screening technologies which allow for more rapid and accurate identification and characterization of therapeutic peptides from random libraries. As described herein, these advantages include improvements in the manner in which the peptide libraries are generated, such that they can be more easily and accurately screened, as well as improved strategies for screening the libraries.

Accordingly, in one aspect, the present invention provides methods for generating random peptide aptamer libraries which are modified to have a desired property, such as improved stability, intrinsic structural organization, affinity, bioavailability, or detectability. In one embodiment, this is achieved by introducing a sequence within the peptide that contributes to the desired property by, for example, cyclizing the peptide. Preferred cyclizable sequences include, for example, those derived from naturally occurring cyclic proteins, such as conotoxins. In another embodiment, the peptide is covalently linked to a cholesterol or sterol, which can direct it to a specific intracellular location or to a metabolic or signaling pathway within the cell.

The advantages of the present invention further include improved methods for delivering and expressing the peptide libraries in cells using, for example, improved receptor-mediated gene transfer systems and/or genetic regulatory elements to enhance expression of the peptide libraries, such as chromatin insulator elements. Other advantages of the present invention include more efficient methods for screening the peptide libraries by identifying populations of cells that have undergone a desired phenotypic change due to the activity of a given member of the peptide aptamer library. This allows for screening of a much larger number of random peptide sequences (e.g., >10⁸), and does not require that the identity of the target be known or specified as a pre-requisite for carrying out the screen.

The following is a summary of several particular embodiments of the present invention as described above.

In one embodiment, the present invention provides a method for identifying a peptide aptamer capable of modifying a cell phenotype by a) contacting cells with a library of expressible nucleic acid sequences encoding random peptide aptamers linked to a fusion moiety; b) selecting at least one cell having an altered phenotype compared to the phenotype of the cell prior to the contacting step (a); and c) identifying one or more peptide aptamers expressed in the selected cell. The method can further include the step of amplifying the nucleic acid sequences identified in step c) and repeating one or more times steps a)-c) using the amplified nucleic acid sequences as the library of expressible nucleic acid sequences. Preferably the nucleic acid sequences are amplified using the polymerase chain reaction (PCR) and a thermostable nucleic acid polymerase.

In one embodiment, the fusion moiety is a green fluorescent protein (GFP), thioredoxin, a regulatory polypeptide involved in apoptosis, bcl-2, p53, an NFκB-related polypeptide, a caspase, PTEN, myc, a BH3 domain, a death domain (DD), a BIR3 domain, a BIR domain, a nuclear localization signal sequence, a membrane localization signal sequence, a farnesylation signal sequence, a transcriptional activation domain, a transcriptional repression domain, or a functional fragment thereof.

In another embodiment, the fusion moiety is a moiety that undergoes processing (processing-fusion moiety) such as an intein or sterol conjugation domain (e.g., hedgehog derived polypeptide), in which processing of the full length precursor results, respectively, in a cyclic or sterol-conjugated peptide product.

The method can further include the step of expressing the one or more peptide aptamers identified in step c) in a second sample of cells (which can be the same or a different cell type as the first sample), and identifying a change in the phenotype of the second sample of cells.

In another embodiment of the invention, the cells of the first sample discussed above are genetically modified to express a receptor, such as ecotropic receptor, not naturally expressed by the cells (or not abundantly expressed endogenously), to facilitate cellular uptake of the nucleic acid sequences or vectors encoding the random peptide aptamers.

In yet another embodiment of the invention, the methods discussed above further include contacting a sample of cells with a pathogen, for example, a virus or toxin, before or after expression of the one or more peptide aptamers. In another embodiment, a sample of cells is contacted with an agent which stimulates signal transduction before or after expressing the one or more random peptide aptamers.

In yet another embodiment of the invention, cells expressing the random peptide libraries are screened by identifying a change in the expression levels of an endogenous gene or gene products. In a related embodiment, the cells contain a reporter gene such that a change in the expression levels of the reporter gene are measured.

The screening methods of the present invention summarized above are used to identify peptide aptamers capable of altering a particular cell phenotype. Such phenotypes include, for example, a change in levels of apoptosis, signal transduction (e.g., cellular responses mediated by a tyrosine kinase or a G protein coupled receptor), protein trafficking, cell adhesion, membrane transport, cell motility, metabolic state, survival or susceptibility to an infectious agent or toxin, or differentiation, as compared, for example, to a control cell. The methods can further be used to identify peptide aptamers capable of altering cellular lifespan or differentiation, hormone production, or metabolic processes such as cholesterol, sterol, or carbohydrate metabolism. Such peptides or aptamers include those relating to, cellular processes associated with hair growth or hair loss (e.g., propecia). Such peptides include those involved in modulation of hair growth, modulation of hair follicle differentiation, modification of hair follicle development, melanogenesis, modulation of hair shaft elongation, modulation of skin differentiation, or a modulation of the hair growth cycle from telogen (resting stage) to anagen (growing stage).

Suitable cells for use the above-described screening methods of the present invention include, for example, fungal cells (e.g., yeast cells), insect cells, and mammalian cells, preferably human cells, and more preferably, clonal human cancer cells, or cells modified to exogenously express receptors or effectors of signal transduction, or cells that exhibit a hallmark of cellular differentiation, for example skin cells suitable for the study of hair growth or loss. The cells are transduced with a library of expressible nucleic acid sequences encoding random peptide aptamers that can be encoded, for example, in a eukaryotic expression vector, such as a retroviral vector. In a particular embodiment, the vector (e.g., the retroviral vector) includes an element to enhance or sustain gene expression, such as a chromatin insulator element.

The present invention further provides peptide aptamers identified using the improved methods described above. Peptide aptamers of the invention generally comprise between 5-9 (e.g., 5, 6, 7, 8, or 9) amino acid residues or more. In one embodiment, the peptide aptamers are fused to an additional amino acid sequence, such as an intein, green fluorescent protein (GFP), a hedgehog polypeptide or functional derivative thereof, thioredoxin, a regulatory polypeptide involved in apoptosis, bcl-2, p53, an NFκB-related polypeptide, a caspase, PTEN, myc, a BH3 domain, a death domain (DD), a BIR3 domain, a BIR domain, a nuclear localization signal sequence, a membrane localization signal sequence, a farnesylation signal sequence, a transcriptional activation domain, a transcriptional repression domain, or a functional fragment thereof.

In a related embodiment, where the fusion moiety is an intein, suitable functional intein moieties are linked to each end of the peptide aptamer so that the peptide aptamer is flanked by intein splicing elements capable of catalyzing the formation of a cyclic peptide aptamer. The inteins flanking the peptide aptamer can further comprise a sequence which can be induced to join the inteins, such that the intervening peptide aptamer is cyclized in the presence of an inducing agent.

Peptide aptamers of the present invention can also include sequences derived from conotoxins which allow for cyclization of the peptide and enhance its ability to be efficiently and accurately screened. Such peptides are referred to herein as “conotides.” In a particular embodiment, the conotide can be characterized by one of the following structural formulae:

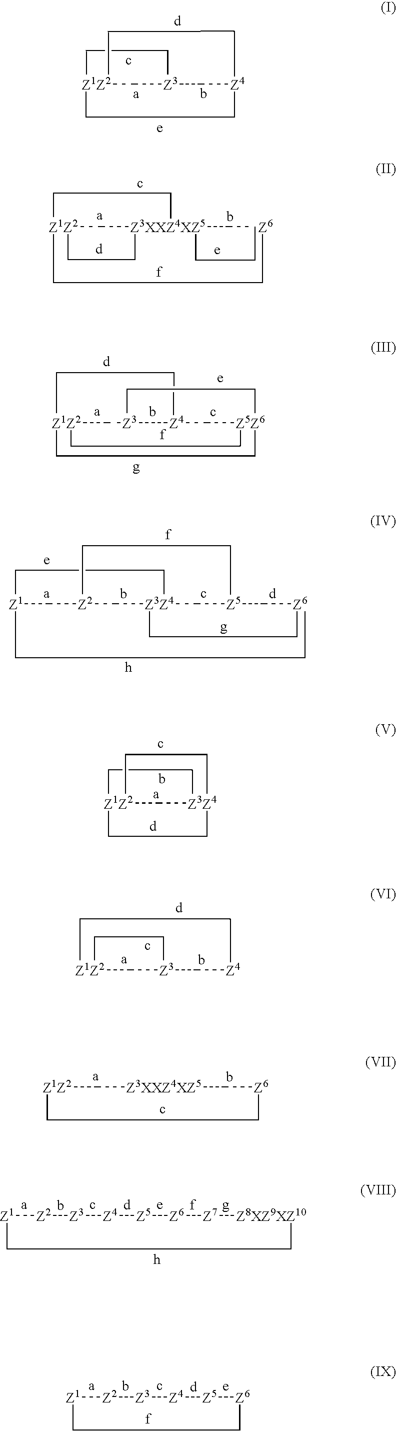

wherein Z¹, Z², Z³, Z⁴, Z⁵, Z⁶, Z⁷, Z⁸, Z⁹, and Z¹⁰ are each independently selected linking residues as described below; and a, b, c, d, e, f, g, and h are each polypeptide linkers as described below. In a particular embodiment, the linkage (Z) is produced by intein-mediated splicing.

In still another embodiment, the invention provides a kit for screening a library of expressible nucleic acid sequences encoding peptide aptamers, conotides, or a panel of peptide aptamers or conotides, optionally with instructions for use.

Peptide aptamers and conotides of the present invention can be used according to art recognized techniques for the molecular modeling of an agent having similar structural and/or functional characteristics as the identified peptide aptamer. The peptides can also be used to treat a disease or condition associated with an aberrant (e.g., misregulated) cell phenotype in a subject by administering to the subject, a therapeutically effective amount of the peptide, or a corresponding expressible nucleic acid (e.g., by gene therapy). The misregulated cell phenotype can be associated, for example, with altered apoptosis, signal transduction, protein trafficking, cell adhesion, membrane transport, cell motility, differentiation, metabolic state, or a disease or condition such as cancer, neurodegenerative disease, viral infection or pathology (for example, due to an toxin), or a disorder associated with cellular differentiation, such as hair growth or loss.

Other features and advantages of the invention will be apparent from the following detailed description and claims.

### BRIEF DESCRIPTION OF THE DRAWINGS

FIG. 1 is an outline of the iterative phenotypic screens encompassed by the invention.

FIG. 2 is an outline of the cyclic peptide libraries, and their structural and functional features, suitable for phenotypic screening and analysis, encompassed by the invention.

FIG. 3 is a schematic illustrating the generation and screening of cyclic peptide libraries in cells using intein fusion moieties that allow for protein splicing and cyclization of the peptide aptamer in cells.

FIG. 4 is an outline of the conotide libraries, and their structural and functional features, suitable for phenotypic screening and analysis, encompassed by the invention.

FIG. 5 is a schematic illustrating the generation and screening of cholesterol or sterol-conjugated protein or peptide libraries through the use of hedgehog catalytic domains.

FIG. 6 is a schematic illustrating the construction and use of a retroviral library to generate random peptide libraries for expression and phenotypic screening in mammalian cells. Nucleotide sequences encoding random amino acid residues are cloned into a retroviral vector for expression either as independent entities, or as fusions to other functional domains such as a functional self-processing domain derived from an intein or sterol-conjugating protein.

FIG. 7 is a schematic illustrating intein-mediated protein splicing (panel A) and the functional residues found within the I_{N} and I_{C} elements of inteins that are directly involved in the splicing reaction and required for activity (panel B).

FIG. 8 shows an alignment of the functional domains within naturally occurring inteins and a representative consensus sequence thereof.

FIG. 9 shows a schematic of the chemistry of intein-mediated protein splicing whereby a cyclized peptide aptamer is produced.

FIG. 10 shows a schematic of inducible intein-mediated protein splicing whereby the intein domains are inactive when fused to interaction domains Prot1 and Prot2 however, in the presence of a ligand, reassociate through Prot1 and Prot2 which restores protein splicing activity such that a cyclic peptide aptamer is produced.

FIG. 11 shows the sequence of an exemplary sterol conjugating protein (hedgehog catalytic domain), peptide aptamer cloning site, and the Gly-Cys-Phe-Pro-Gly-Ser residues necessary for cleavage and sterol conjugation reactions to occur.

FIG. 12 shows a schematic (panel A) and corresponding ribbon structure (panel B) of a constrained epitope (Myc) fused to GFP which is generated upon intein-mediated protein splicing.

FIG. 13 shows the structure of a representative conotoxin (α-conotoxin) with free amino- and carboxy-termini and cysteine-mediated intramolecular disulfide bonds (panel A) and as fusion imbedded between self-splicing intein domains (I_{N}, I_{C}) before and after protein splicing (panel B).

FIG. 14 shows a schematic of the screening method of the invention for identifying a peptide aptamer that alters a cell phenotype, in particular, an aptamer capable of causing apoptosis (cell death) in human leukemic cells (HL60) but not in normal cells.

FIG. 15 shows a schematic of how the screening methods and peptide aptamers of the invention can be used to identify aptamers capable of altering a cell phenotype specific for a particular cancer cell which can then be tested in diverse panel of other cancer cell types to assess its range of action, such that an “aptamer profile” for each type of cancer can be assembled (see text).

FIG. 16 shows a schematic of a screening method for identifying aptamers having antiviral activity such as the ability to block viral infection, viral replication, viral proliferation, or viral induced lysis or apoptosis.

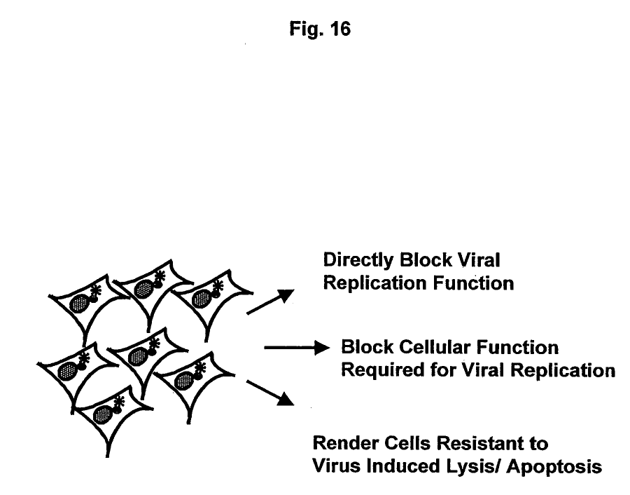

FIG. 17 shows a map of the pIMPS retroviral expression vector which allows for the convenient cloning of a nucleic acid or library of nucleic acids encoding peptide aptamer sequence(s) in frame with adjacent intein domains (I_{C} and I_{N}) for expression and intein-mediated peptide cyclization in cells.

FIG. 18 shows a map of the an altered form of the pIMPS vector, in which a histidine leader epitope tag (6×HIS) is expressed at the carboxy-terminus of the intein flanked peptide for detection and/or quantitation, of either the unspliced or cyclic form of the peptide aptamer, by immunoblot or immunofluorescence.

FIG. 19 shows a digital image of an immunoblot showing intein-mediated splicing of a test cyclic peptide (TCP) or green fluorescent protein (GFP), as compared to controls (i.e., non-splicing (ns) forms), when expressed in mammalian cells.

FIG. 20 shows a digital image of an immunoblot showing that the substitution of alanine residues (AA) for Gly-Pro β-turn structures within a test cyclic peptide (TCP) decreases the efficiency of intein-mediated protein splicing.

FIG. 21 shows a strategy for demonstrating constraint of epitopes presented by linking the amino and carboxy-termini of GFP over the “Top” of the cyclized GFP barrel structure.

FIG. 22 shows a digital image of an immunoblot showing aptamers constrained over the “Top” of a cyclic GFP protein when expressed in mammalian cell extracts.

FIG. 23 shows a digital image of an immunoblot showing the expression and intein-mediated splicing of a test cyclic peptide (TCP) and three different conotides when inducibly expressed in bacteria (i.e., in presence (+) or absence (−) of IPTG). The lower panel shows the amino acid sequences of the test cyclic peptide (TCP) and representative conotides (CGRH, SCGS, and CCNP) expressed.

FIG. 24 shows a schematic of how a sterol conjugation protein (catalytic domain of hedgehog) can be fused to a peptide aptamer (panel A) and, when expressed in cells and in the presence of a sterol(s), yield a sterol conjugated peptide sequence clipped free of the sterol conjugation protein (panel B).

FIG. 25 shows a digital image of an immunoblot of cell extracts showing that a sterol conjugation protein (catalytic domain of hedgehog) can be fused to a test protein (GFP) and, when expressed in cells in the presence of a sterol, yield a sterol conjugated test protein clipped free of the sterol conjugation protein (hedgehog). “−” represents constructs containing alanine substitutions preventing cleavage; “SG” represents constructs with an additional Ser-Gly spacer to improve cleavage; and “*” indicates native test protein (GFP) which migrates faster than the conjugated GFP.

FIG. 26 shows a diagram of the pcDNA4-ATRC-1 expression vector containing a cDNA encoding the mouse ATRC-1 receptor and a selectable marker for zeomycin resistance.

FIG. 27 shows FACS results (panel A), and a graphical representation thereof (panel B), demonstrating that overexpression of the mouse ATRC-1 receptor in murine cells dramatically increases their susceptibility to transduction using a recombinant ecotropic retroviruses, as compared to a control.

FIG. 28 shows a schematic of a strategy for selection and identification of activators of the erythropoietin receptor signal transduction pathway.

FIG. 29 shows digital images of cells expressing two different test proteins (i.e., GFP and activated Ras) fused to a sterol conjugation domain (catalytic domain of hedgehog ). GFP fused to a sterol conjugation domain remains functional (fluoresces) when expressed in cells and sterol conjugated in the presence of a sterol (top panels). Activated Ras (V12Ras) transforms cells in which it is expressed (middle left panel) but not if missing a membrane targeting domain (CAAX domain) (middle right panel). If activated V12Ras without a CAAX membrane targeting domain is fused to a sterol conjugation domain, the membrane targeting and transforming function of the mutant Ras protein is restored (lower left panel). This is dependent on a functional sterol conjugation domain (lower right panel).

### DETAILED DESCRIPTION OF THE INVENTION

The present invention provides improved methods and compositions for screening and identifying peptide aptamers (e.g., from random libraries) that manifest identifiable phenotypes in cells. This includes not only the generation of peptide aptamer libraries, but also the delivery of the libraries into cells and the screening of the libraries by identifying alterations in a cell phenotype. Accordingly, the invention shall be described below under the following four (4) subheadings: 1) the making of peptide aptamers (libraries), 2) delivery and expression within cells of nucleic acids encoding peptide aptamers, 3) screening and identifying peptide aptamers capable of altering a cell phenotype, and 4) use of peptide aptamers for the prognosis, diagnosis, and/or treatment of a disease or disorder.

In describing the invention, the following definitions shall be applied:

Definitions

As used herein, the term “aptamer” or “peptide aptamer” refers to a polypeptide, generally between 2-40, preferably between 5-20 (i.e., 5, 6, 7, 8, 9, 10, 11, 12, 13, 14, 15, 16, 17, 18, 19, or 20), most preferably between 5-10 (e.g., 7) amino acid residues in length, capable of modifying a cell phenotype, e.g., an observable characteristic of a cell, when introduced into or expressed in the cell (or organism). The polypeptide may be linear or cyclic. In a particular embodiment, the peptide aptamer exists intracellularly in a cyclic form generated through intein-mediated processing, or covalently conjugated to cholesterol or derivatized sterols generated through hedgehog-mediated processing (or functional derivative thereof). In another embodiment, the peptide aptamer comprises a sequence derived, in whole or in part, from a conotoxin peptide. Such peptide apatamers are referred to herein as “conotides.” Cyclic peptides aptamers of the invention may also comprise one or more linking moieties. The peptide aptamers of the invention can be expressed intracellularly from a corresponding nucleic acid or produced (e.g., synthesized) and used extracellularly (e.g., for binding to a cell surface receptor) or intracellularly by introducing the peptide into a cell using art recognized techniques.

The term “conotide” refers to a class of peptide aptamers which include sequences and/or structural motifs derived from, or characteristic of, conotoxins. Conotoxins are naturally occurring, stable, and typically cyclic polypeptides having a range of biological activities, including, for example, modulating neuronal signals. The biological activity of conotoxins depends upon a looped structure that occurs through two or more cysteine-mediated intramolecular disulfide bonds. Accordingly, conotides of the invention are engineered to include sufficient conotoxin sequences such that, for example, the conotide exhibits improved stability, a cyclic structure (i.e. a contiguous, cyclic polypeptide backbone), or improved biologic activity. The conotoxin-derived portion of the conotide is typically selected to provide for the cyclization of the peptide aptamer through one or more linkages, and may include, e.g., the modification of one or more conotoxin derived residues, e.g., cysteines, with a different amino acid or modified amino acid. The conotoxin-derived sequence of the conotide also may be substantially modified such that no original conotoxin-derived amino acid sequence remains but the spacing, or a similar spacing, of one or more cyclic linkages from the conotoxin does remain. Conotoxin-derived sequences may be derived from any known conotoxin protein, for example, thos included within the conotoxin superfamilies A, M, O, S, T, P, or non-disulfide rich conotoxins such as, e.g., conantokin, contulakin, or contryphan (see, e.g., Table 1).

The term “conotide library” refers to a group of two or more conotides or conotide-encoding nucleic acids.

The term “random” refers to differing sequences within the peptide aptamer which are not predetermined. Accordingly, a “random peptide aptamer” contains in whole, or in part, random sequences. In certain embodiments, the peptide aptamer further includes certain predetermined sequences, e.g., to impart stability or secondary structure to the polypeptide. For example, certain peptide aptamers, such as conotides, typically contain a discontinuous random sequence that is interrupted by one or more predetermined or semi-random residues, such as linking residues. When referring to a codon, residue, or peptide, the term “random” means that, the codon, residue, or peptide may consist of any series of nucleotides or amino acids, except in the case of a stop codon which does not encode a residue and would result in an incomplete peptide aptamer.

The term “fusion moiety” refers to a polypeptide sequence which is linked (e.g., fused) to a peptide aptamer to improve its stability, affinity, bioavailability, visualization, detection and/or cause localization of the aptamer. Suitable fusion moieties include, but are not limited to, intein sequences (which allow for the cyclization of the peptide aptamer through intein-mediated protein splicing), hedgehog polypeptides and functional derivatives thereof (which can become linked to a sterol for membrane targeting of the peptide aptamer), localization sequences (e.g., membrane localization sequence or nuclear targeting sequence which allow for targeting of the peptide aptamer to a particular cellular location), detectable marker proteins (e.g., GFP (green fluorescent protein) or luciferase, which allow for visual detection of the peptide aptamer).

The term “processing-fusion moiety” refers to a subset of fusion moieties capable of being processed, e.g., undergoing a catalytic cleavage reaction, and includes, e.g., intein splicing moieties and sterol-conjunction moieties (e.g., hedgehog derived proteins).

The term “hedgehog polypeptide” refers to any member of the class of hedgehog polypeptides (e.g., mammalian, murine, or Drosophila), or derivative or homolog thereof, which has autoprocessing activity and the ability to mediate conjugation to a sterol, e.g., cholesterol. The hedgehog polypeptide (or derivative thereof) may be produced by purifying from a native source, recombinantly produced, or synthesized. The hedgehog polypeptide can be modified, even substantially so, as long as its autoprocessing activity and the ability to become conjugated to a sterol remains intact. The catalytic domains for a number of hedgehog polypeptides, i.e., the autoprocessing and/or sterol conjugation domains, have been described.

The term “peptide” or “polypeptide” and “protein” are used interchangeably throughout the specification and refers to two or more amino acid residues linked by a polypeptide bond.

The term “library of expressible nucleic acid sequences encoding random peptide aptamers” refers to a collection or plurality of nucleic acid sequences that encode different peptide aptamers (either alone or linked to a fusion moiety). Peptide aptamers differ randomly by one or more amino acids. Typically, the nucleic acid sequences are contained within a vector, for example, a plasmid, that can be propagated in a host cell, e.g., a prokaryotic host, and can also be used to transfect or infect a eukaryotic cell. It is understood that the invention also encompasses peptide aptamer libraries, such as a collection of random peptide aptamers or conotides (or both), which can be produced recombinantly or synthetically and may exist in solution or in solid phase, for example, bound to a matrix, chip, or array.

The terms “vector”, “vector construct”, “expression vector”, and “plasmid” are used interchangeably. The term “vector” also includes viral vectors, such as retroviral vectors derived from retroviruses, adenoviruses, SV40, adeno-associated viruses, or other suitable viruses recognized in the art.

The term “phenotype” includes any observable characteristic of a cell or organism. Thus, phenotypic changes selected for in the present invention include both histological changes which can be visually determined, as well as functional changes (including, e.g., measurable changes in gene expression). Phenotypic changes also include a change in the ability of the cell or organism to undergo, e.g., undesired cell growth, or survive a viral infection or a change of the ability of an infecting virus to replicate within the cell. The term phenotype also includes, for example, a change in levels of apoptosis, signal transduction (e.g., cellular responses mediated by a receptor or a kinase, e.g., a cell surface receptor, nuclear receptor, or G protein coupled receptor, or a tyrosine kinase or serine and/or threonine kinase), protein trafficking, cell adhesion, membrane transport, cell motility, metabolic state, survival or susceptibility to an infectious agent or toxin, or differentiation, as compared, for example, to a control cell. In particular, the term phenotype includes a change in cellular lifespan or differentiation, hormone production, or metabolic processes such as cholesterol, sterol, or carbohydrate metabolism, or cellular processes associated with hair growth or hair loss (e.g., propecia).

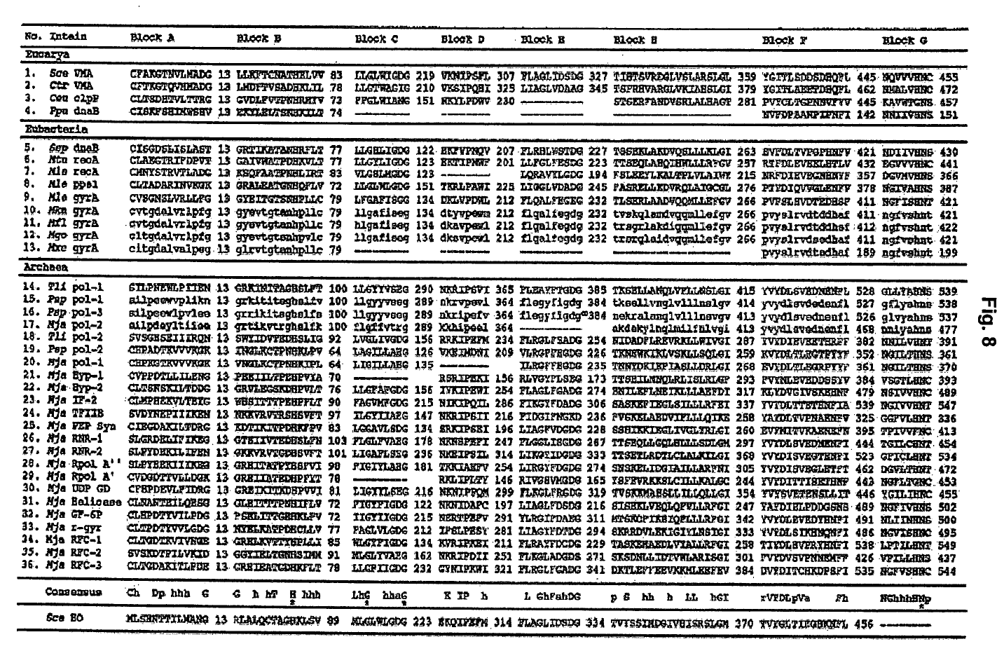

The term “intein” or “intein domain” includes any amino acid sequence which can undergo a protein/protein splicing reaction such that excision and/or cyclization of the amino acid or a linked amino acid sequence occurs. Accordingly, the term intein includes naturally-occurring or artificially engineered polypeptide sequence that can catalyze a protein/protein splicing reaction. A list of known inteins is published at http:www.neb.com/inteins.html and a partial list showing relevant consensus sequences is shown in FIG. 8 (see also Perler et al., NAR, 25:1087-1093 (1997)).

The term “split intein” includes an intein that has two or more separate regions which can catalyze a protein/protein splicing reaction but which are separated by an intervening sequence, e.g., a protein of interest, e.g., a random peptide aptamer.

The term “intein-mediated” includes a polypeptide (e.g., aptamer) splicing or cyclization event that is catalyzed, at least in part, by an intein sequence, e.g., an intein domain.

The term “protein splicing” includes the post translational cleaving and rejoining of a polypeptide.

The term “cyclization” refers to any chemical or enzymatic reaction which results in the cyclization of an amino acid sequence, preferably, e.g., via a covalent bond.

The term “cyclic peptide”, “cyclic polypeptide”, or “cyclic protein” refers to a peptide or polypeptide sequence where at least a portion of its primary sequence is cyclized.

The term “retroviral vector” includes a vector containing, at least in part, retroviral sequences, which is capable of delivering a nucleic acid sequence (e.g., a peptide aptamer encoding sequence) to a cell (e.g., eukaryotic cell). Retroviral vectors (encoding a library of peptide aptamers) can be generated, for example, by transfecting a pre-constructed plasmid library into an appropriate retroviral packaging cell line.

The term “screen” includes an assay which comprises one or more parameters (or measurement thereof) to identify the presence of a particular aspect (e.g., genotype or phenotype), whereas a “counterscreen” holds constant the foregoing aspect in the presence or measurement of other parameters to validate the predetermined aspect.

The term “phenotypic selection” refers to the assaying of any observable characteristic (including any measurable gene expression levels using, e.g., gene array chips) of a cell or organism, e.g., a cell or organism expresses or is contacted with one or more aptamers (e.g., conotides) of the invention.

The term “linked”, when referring to nucleic acids, includes the joining of two or more nucleotide sequences where each encodes an open reading frame and the open reading frame of each is preserved such that a genetic fusion polypeptide results. Accordingly, the term “linked”, when referring to protein sequences, means conjugated as a single fusion protein, or embodied forms of intramolecular chemical conjugation.

The term “linking moiety” includes moieties which link non-adjacent amino acids (e.g., linking residues) of the conotides of the invention. The linking moieties link the amino acid linking residues through covalent linkages (e.g., carbon-carbon bonds, polypeptide linkages, disulfide bonds, thioether bonds, ether bonds, etc.), hydrophobic linkages (e.g., aromatic stacking, e.g., tryptophan stacking), or ionic linkages (e.g., salt bridges, hydrogen bonds, etc.).

The term “linking residues” (“Z”) includes one or more attaching moieties covalently bonded to a backbone (e.g., an amino acid residue) of the conotides of the invention. The linking residue may be a natural amino acid (e.g., cysteine or tryptophan) or it may be chemically modified (e.g., to incorporate specific attaching moieties in the side chain) such that it is capable of performing its intended function. Methods for modifying side chains of amino acids are known in the art.

The term “attaching moiety” includes moieties which are capable of being linked (e.g., independently or through the use of a linking reagent) to another attaching moiety (which may be the same or different) such that a linking moiety is formed. For certain linkages (e.g., polypeptide linkages), the attaching moiety may be the C- or N-terminus of an amino acid.

The term “ionic linkages” includes ionic interactions between attaching moieties which enhance the stability, structure, or activity of the peptide aptamer, e.g., conotide and, preferably, do not inhibit the peptide aptamer or conotide from performing its intended function. Attaching moieties suitable for forming ionic linkages (“ionic attaching moieties”) include, but are not limited to, charged species such as ammonium, and species capable of forming hydrogen bonds (e.g., sulfate, sulfite, phosphate, phosphite, nitrate, nitrite, SH, OH, NH₂, etc.). Generally, the ionic attaching moieties are attached via covalent bonds to a linking residue.

The term “hydrophobic linkages” includes interactions between attaching moieties which are mediated by, for example, van der Waals or other hydrophobic attractions, such as aromatic or tryptophan stacking, which may confer structural features, or such that the stability or activity of the conotide is not inhibited, and is preferably enhanced. Preferably, peptide aptamers or conotides comprising hydrophobic linkages have increased structural and chemical stability in vivo, as compared to such peptides without these linkages. Examples of attaching moieties which interact through hydrophobic interactions include, but are not limited to, aromatic groups capable of aryl stacking, such as the side chains of tryptophan, phenylalanine or tyrosine.

The term “covalent linkages” includes covalently bonded chains of one or more atoms, which covalently link linking residues. The atoms of the chain may be, but are not limited to, carbon, oxygen, nitrogen, boron, phosphorous, sulfur, silicon, etc. In certain embodiments, covalent linkages may require one or more linking reagents for formation. The covalent linkage is generally formed through the interaction and chemical reaction of two or more attaching moieties and any necessary linking reagent, such that the covalent linking moiety is formed. Examples of attaching moieties for covalent linkages include, but are not limited to, alkenes, alkynes, amines, carboxylic acids, halogenated aromatics, aldehydes, and vinyl halides. Covalent linkages may be synthesized using, for example, transition metal catalyzed cross couplings, pinacol couplings, hydrozirconation, olefin metathesis, nucleophilic addition, aldol/Curtius rearrangements, and NBK coupling (Nozaki-Hlyama-Kishii,'Fiirstrier et al., J Am. Chem. Soc. 1996, 118, 12349)). In a further embodiment, the covalently bonded chains of atoms in a particular covalent linkage has zero, one, two, three, four, five, six, seven, eight, nine, ten or more atoms in the chain, independent of any substitutions or cyclic moieties. In certain embodiments, such as when the linking residues of the covalent linkage are each cysteine, such as conotide derived from a naturally occurring conotoxins having such residues, the covalent linkage is not a disulfide linkage.

The term “polypeptide linkages” includes a covalent polypeptide linkage having at least one peptide bond. For example, the polypeptide linkages include moieties which comprise at least one peptide bond. Furthermore, the polypeptide linkages may comprise one or more amino acid residues connected through peptide bonds. The number of amino acid residues present in a particular polypeptide linkage may be selected such that the peptide aptamer or conotide manifests a specific function. The number may be selected for each occurrence separately. In certain embodiments, the polypeptide linkages may comprise zero, one, two, three, four, five, six, seven, eight, nine, ten, eleven, twelve, thirteen, fourteen, or more amino acid residues. In another embodiment, the polypeptide linkage is produced through intein-mediated protein splicing.

The term “membrane transport” includes facilitated movement of a determined ion, protein, nucleic acid, or class of molecules across the plasma, mitochondrial or nuclear membrane of a cell.

The term “sterol” or “sterol derivative” includes any compound which contains a sterol functional group and can be conjugated to a polypeptide by a hedgehog polypeptide as defined above (e.g., 1,7-dehydrocholesterol (5,7-cholestadien-3β-ol), 5-androsten-3β-ol, desmosterol; 24-dehydrocholesterol (5,24-cholestadion-3β-ol), β-silosterol (5-stigmasten-3β-ol), 7β-hydroxycholestrol (5-cholestene-3β,7β-dial), and cholesterol (5-cholestene-3β-ol).

The term “secondary library” includes a library that is derived from, or is a subset of, a first library such that the secondary library has a higher probability or bias for containing or encoding a molecule having a desired characteristic. Typically, the secondary library is the product of a selection or screening method using, e.g., a biochemical assay (e.g., a binding assay), cell-based assay, or in vivo assay (e.g., where an animal is administered a library of aptamer peptides, e.g., expressible aptamer peptides in the form of a gene therapy vector which after, e.g., a period of time or challenge, the remaining expressible aptamers are isolated from the animal, e.g., re-cloned or identified from a cell or tissue source).

The term “chromatin insulator” includes a cis-acting sequence within an expression vector that enhances expression of coding sequences within the vector, and which can perpetuate expression throughout passage or differentiation of the transduced cell.

The term “effector” includes a naturally-occurring cell-associated (e.g., endogenous) polypeptide that is, directly or indirectly, responsible for a cellular phenotype.

The term “cancer” includes any neoplasm, such as a carcinoma (derived from epithelial cells) or sarcoma (derived from connective tissue cells) or a cancer of the blood, such as a leukemia.

The term “viral resistance” includes the level of infection or cytopathic effect measured after a cell is infected, transduced, or otherwise invaded by a virus.

The term “apoptosis” includes any non-necrotic, cell-regulated form of cell death, as defined by criteria well established in the art.

The term “cell” includes any prokaryotic or eukaryotic cell. A eukaryotic cell can be a fungal cell (i.e., yeast cell), insect cell (e.g., Schneider and sF9 cells), or somatic or germ line mammalian cell, or cell line e.g., HeLa cell (human), NIH3T3 (murine), RK13 (rabbit) cells, an embryonic stem cell (e.g., D3 and J1), and cell types such as a hematopoietic stem cell, myoblast, hepatocyte, lymphocyte, and epithelial cell and, e.g., one of the following cancer cell lines. HL-60, HeLa, HepG2, Molt-4, NCI-H23, DMS114, HCC-2998, U251, SK-MEL-28, SK-OV-3, A498, PC-3, and SK-BR-3.

The term “surrogate signaling molecule” refers to a molecule that brings about a cellular response that phenotypically mimics the response of a cell to ligand engagement of a specific signal transducing receptor, but which is brought about in the absence of a ligand that would normally manifest that response. In addition, a surrogate signaling molecule may increase the sensitivity of a cell to receptor-ligand engagement, thereby allowing response to contacting the receptor and ligand, but at ligand concentrations lower than those necessary for the activity of the ligand in the absence of a surrogate signaling molecule.

The term “predetermined” refers to aptamer sequences that are designed to have particular non-random amino acids or motifs.

The term “selecting” refers to the identifying of cells phenotypically or functionally, e.g., as compared to control cells, as well as cells that are physically selected (e.g., cell sorting, survival).

The term “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. Methods for generating such transgenic animals (e.g., via embryo manipulation and microinjection), particularly animals such as mice, are well known in the art as described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, Second Edition (Cold Spring Harbor Laboratory Press, Cold Spring Harbor, N.Y., 1994).

I. Generation of Peptide Aptamers

1.1 Generation of Peptide Aptamers and Libraries Thereof

The peptide aptamers of the invention contain random combinations of a small number of amino acid residues, e.g., 5, 6, 7 or more, but preferably less than 100, more preferably less than 50, and most preferably less than 20. The peptide aptamers of the invention can be produced recombinantly, from a corresponding nucleic acid sequence, or synthetically using art recognized techniques in peptide chemistry. A library of nucleic acid sequences encoding random peptide aptamers can be generated by combinatorial mutagenesis at the nucleic acid level. Alternatively, a variegated library of nucleic acid sequences encoding random peptide aptamers can be produced using chemical synthesis of a degenerate aptamer gene sequence using an automatic DNA synthesizer, where the synthetic gene is then ligated into an appropriate expression vector, e.g., a retroviral vector. Use of a degenerate set of genes allows for, in one mixture, all of the sequences encoding the desired set of potential random peptide aptamer sequences to be represented. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

1.2 Generation of Peptide Aptamers Linked to Fusion Moieties

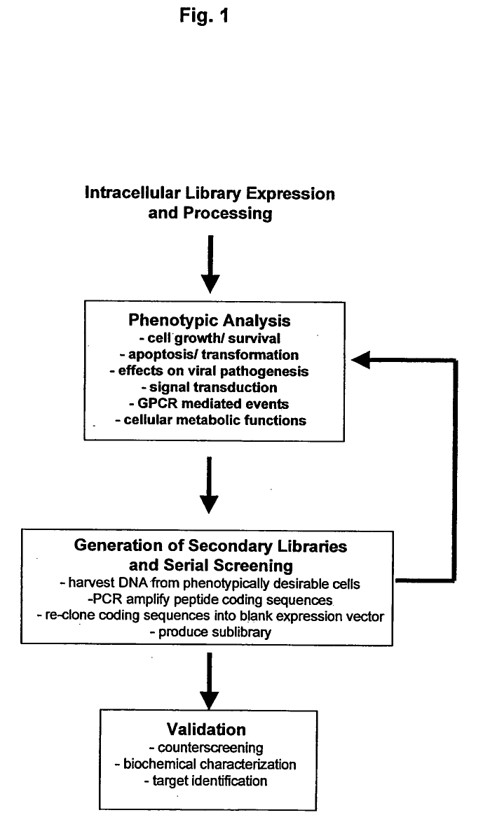

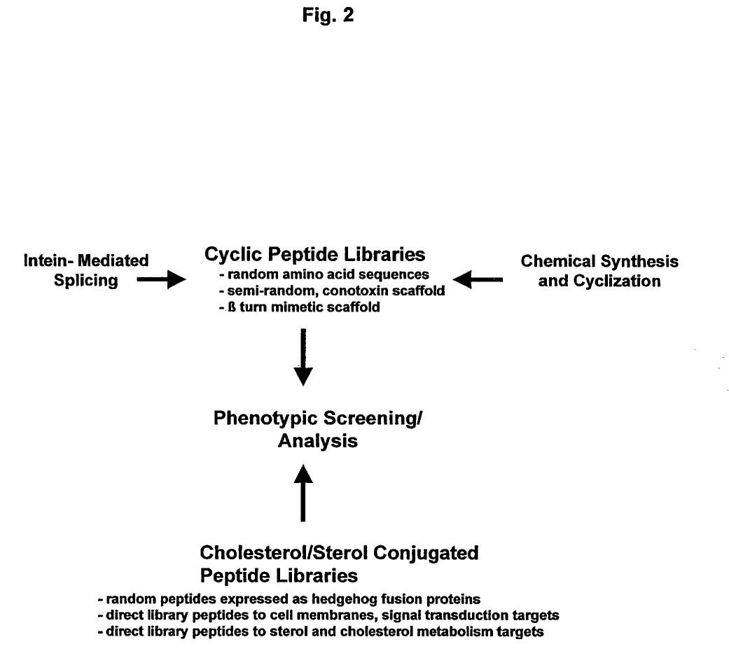

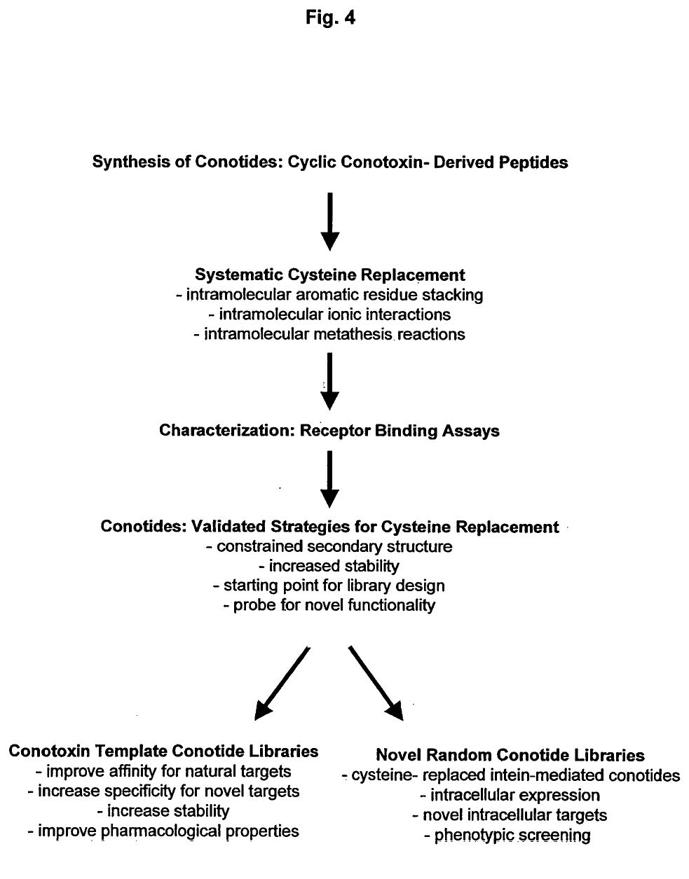

The peptide aptamers of the invention can be produced as free entities, or depending on the target of a given screen, as fusions to a heterologous protein, such as a protein, interchangeably referred to as a “protein scaffold” or “fusion moiety”, e.g., “processing-fusion moiety”, that can promote, e.g., expressibility, intracellular cyclization, or modification, localization, stability, secretability, isolatablitiy, or detectability of the peptide aptamer (see, e.g., FIGS. 1-4).

Fusion moieties can be “neutral” moieties that allow monitoring of expression (e.g. a catalytic or detectable moiety such as chloramphenicol acetyltransferase, β-galactosidase, or green fluorescent protein). Alternatively, fusion moieties that encode targeting domains such as a nuclear or membrane localization signals, transcriptional activation or repression domains, a CAAX farnesylation signal sequence that directs membrane localization, MHC proteins, SH2, or SH3 domains, BH3, DD, BIR3, or BIR domains, can be used to direct peptide aptamers to other functional or regulatory cellular circuits. Still further, the fusion moieties can constrain the peptide aptamer, by for example, reducing flexibility, e.g., rotational degrees of freedom (e.g., using a proline), allowing flexibility (e.g., using a Gly-Pro or Pro-Gly to increase the β-turn character of the peptide and facilitate cyclization), and/or providing certain residues, residue positions, and/or linkages that allow for intrapeptide bonds or cyclic backbone structures to be formed (e.g., as contained within a conotide as described below).

The fusion moieties that constrain or loop a aptamer peptide thereby making it cyclic are desired in such phenotypic screens, because of the intrinsic structural organization they impose upon the peptide (e.g., the backbone of the peptide is not free to swivel and allow for many transient conformations) and because cyclic peptide aptamers are much more stable than linear peptides (e.g., have a longer intracellular half-life). Naturally-occurring peptides with looped structures often contain cysteine-mediated intramolecular disulfide bonds, which are not stable in the reducing environment of the mammalian cell cytoplasm. Thus, the peptide aptamers of the invention overcome the limitations of other strategies that use linear or looping sequences without cyclization by forming stable covalently closed peptide aptamer loops within the cell. Typically, the resulting peptide aptamer spontaneously cyclizes within the cellular milieu and contain no other sequence except random aptamer sequence or, optionally, a fusion moiety such as, e.g., thioredoxin or GFP. The cells expressing such modified peptide aptamers are then monitored for any phenotypic change as described herein.

Other fusion moieties include processing-fusion moieties (and products thereof) which are capable of catalyzing a protein modification, e.g., splicing of the protein (including inducible splicing) or conjugating the protein to a sterol, and these aspects of the invention are introduced in following three subsections (1.3, 1.4, and 1.5).

1.3 Generation of Cyclic Peptide Aptamers Using Protein Splicing

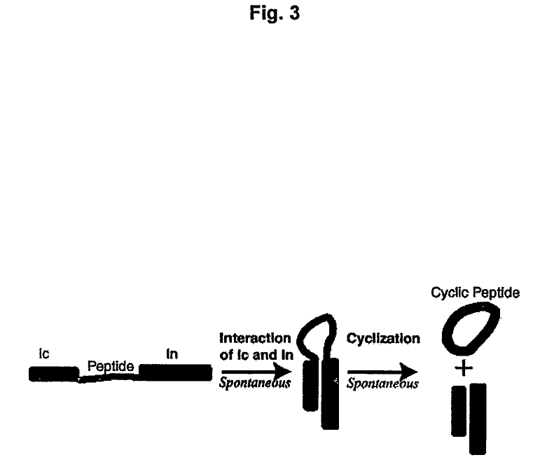

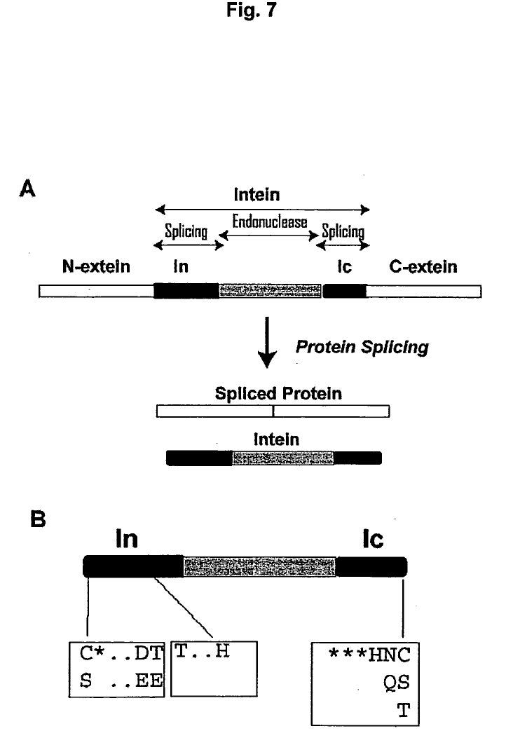

In a particular embodiment, the invention provides for the generation of cyclic peptide aptamers with improved stability using protein splicing. The cyclization of the peptide aptamer is achieved by linking to the peptide aptamer a fusion moiety that contains an intein sequence (or functional derivative thereof) (see FIGS. 3 and 7). Intein sequences are naturally-occurring protein sequences which have been identified in a number of proteins and can catalyze self-splicing of the protein in which they are found (see FIG. 8).

Inteins are the protein equivalent of introns, in the sense that they are internal sequences that are removed from a full-length immature host protein in order to form a functional product. This catalytic reaction results in the excision of the intein and in the formation of a mature host protein. This naturally occurring, spontaneous process is depicted in FIG. 7. Inteins exist in a large number of organisms and so far, all known inteins have been compiled in a database (http://www.neb.com/neb/inteins.html; see also FIG. 8.). Exemplary intein sequences which can be fused to a random peptide sequence include, e.g., those comprising or derived from DnaE from Synechocysti (see e.g. Example 1).

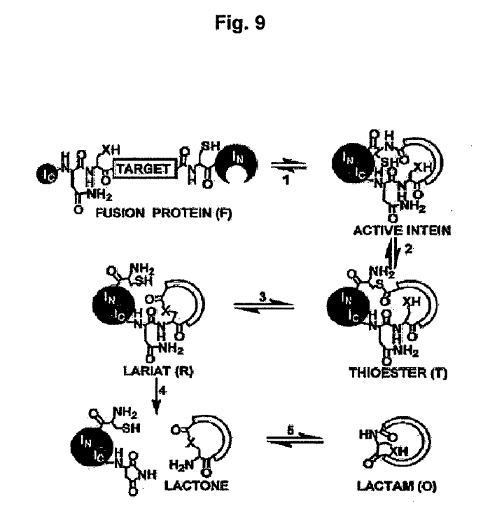

Importantly, inteins can be split to generate cyclic peptides (see, e.g., WO 00/36093). In that case, intein moieties capable of carrying out the intein splicing reaction are split in two parts surrounding the peptide to be cyclized. The chimeric immature product is composed of the carboxyl-terminus half-part of the intein (Int-C), the peptide of interest, followed by the amino-terminus half-part of the intein (Int-N). The association of Int-C and Int-N reconstitutes a functional intein and results in the auto-catalytic excision of the central peptide in a cyclic form (cyc-Pep). The generation of a cyclic peptide as opposed to a linear peptide (lin-Pep) presents several advantages: an increased stability of cyc-Pep compare to lin-Pep due to its potential resistance to peptidases and a conformational stability due to the structural constraints imposed by the cyclization (see FIG. 9).

Accordingly, using genetic engineering techniques, these elements can be used to modify a target sequence such that, when expressed as a polypeptide, the added intein motifs will catalyze a protein splicing reaction (as described, for example in U.S. Pat. No. 5,496,714 and WO 00/36093). Spontaneous self-splicing of the intein yields a cyclized form of the intervening peptide: the product of this reaction is a cyclized peptide in which the boundary features have been extruded during the intein slicing reaction. Accordingly, in one embodiment, the nucleic acids encoding the peptide aptamers are artificially engineered to further encode self-splicing intein elements flanking the intervening random peptide aptamers.

The self-splicing peptide aptamer libraries can be expressed in any eukaryotic cell, e.g., mammalian cells, by means well known in the art, including standard expression plasmids, e.g., retroviral expression vectors. Typically, such libraries contain flanking sequences capable of self splicing the intervening peptide, which is composed of random amino acid sequences of specified length (e.g., at least three, preferably four, five, six, seven or more amino acids).

The vectors encoding the self-cyclizing peptide libraries are then introduced into the test cell, e.g., a mammalian cell, and the cells are subsequently analyzed for acquisition of a specified peptide aptamer-dependent phenotypic change.

1.4 Generation of Cyclic Peptide Aptamers Using Inducible Protein Splicing

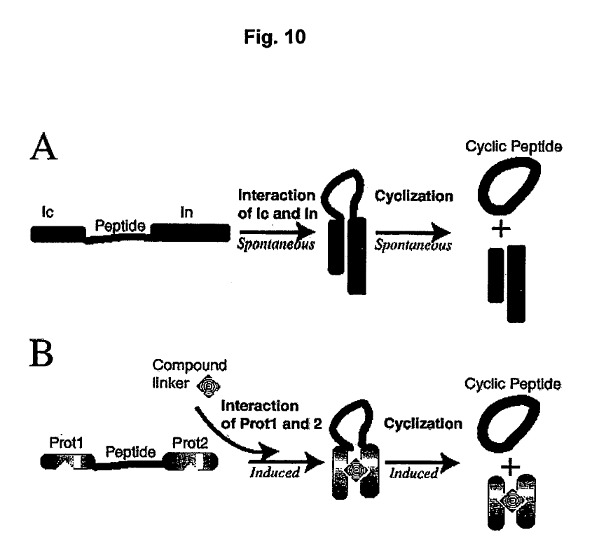

The invention can also be performed using aptamers that are first expressed as inteins which then are processed under inducible control. Normally, the split inteins, as discussed above, undergo cyclic catalysis without regulation. By contrast, the invention provides a system where the formation of the cyc-Pep can be controlled at any time by engineering an intein where the critical residues are retained that participate directly in the chemical reactions leading to peptide backbone cleavage and rejoining, but where the 2 independent components of the split-intein that act to bring together these reactive sites are replaced, in whole or in part, by proteins or domains capable of interacting only under controlled conditions (see FIG. 10). This confers the advantage of inducing the production of cyclic peptides at very defined times and under very defined conditions. Accordingly, an important advantage of this inducible system is the ability to identify cyclic peptides during a biological screen that may have secondary effects or toxicity that otherwise would not normally allow their evaluation or detection.

Inteins have been well studied and the critical residues necessary for the self-splicing catalysis and for the endonuclease activity have been identified (Liu 2000, Paulas 2000, and Perler 1997). Accordingly, the invention provides two polypeptide domains, Prot1 and Prot2, which replace respectively, Int-C and Int-N, for producing cyclic peptides. As opposed to the constitutive splicing activity catalyzed by split-inteins, the catalytic reaction of the engineered intein Prot1/Prot2 is based on the inducible propensity of Prot1 and Prot2 to interact under precise conditions. These conditions can occur by the addition of a chemical compound that promotes the interaction of Prot1 and Prot2 or the induction of a signaling pathway in the cell that results in the covalent modification of either Prot1 or Prot2 which will then induce their binding to each other.

Preferably, Prot1 and Prot2 are selected for their small size; that they do not interact in the absence of the defined conditions or compound; that they do interact with a strong affinity in the presence of the defined conditions or compound; that the compound allowing the interaction of Prot1/Prot2 is preferentially cell permeable and with no additional or strong interfering effect than its binding to Prot1 and Prot2; and that once associated, the C-terminal domain of Prot1 is within a relatively small distance of the N-terminal domain of Prot2.

Accordingly, in one embodiment, the Prot1 and Prot2 is the immunophilin FKBP12 which forms a dimer in presence of the chemical compound FK1012. The dimerization of FKBP12 can be reverted by the monomeric molecule FK506.

In another embodiment, the Prot1 and Prot2, are, respectively, calmodulin and the regulatory domain of the protein kinase CaMPKII, which forms a dimer in the presence of calcium. Release of intracellular calcium can be achieved by, e.g., various art recognized cellular stimuli or by the addition of a calcium ionophore, such as A23187. Calcium levels can also be modulated through calcium chelators.

In another embodiment, Escherichia coli dihydrofolate reductase enzyme (DHFR) binds the ligand methotrexate (MTX) and can form a dimer in presence of bisMTX, a homobifunctional version of MTX. Thus, this domain can be used for the Prot1 and Prot2 domains.

In yet another embodiment, the vitamin D receptor (VDR) binds to NCoR or to Alien and these proteins are used for the Prot1 and the Prot2 domains. The 1,25-dihydroxyvitamin D3 dissociates these complexes and stimulates the transcriptional activity of the receptor.

In still another embodiment, the human growth hormone (hGH) and the extracellular portion of the growth hormone receptor (hGHR) are engineered so that they can no longer interact. Mutations introduced into human growth hormone (hGH) (Thr175 Gly-hGH) and the extracellular domain of the hGH receptor (Trp104 Gly-hGHbp) created a cavity at the protein-protein interface that resulted in binding affinity being reduced by a factor of 10⁶. A small library of indole analogs was screened for small molecules that bind the cavity created by the mutations and restore binding affinity. The ligand 5-chloro-2-trichloromethylimidazole was found to increase the affinity of the mutant hormone for its receptor more than 1000-fold (Guo et al., 2000). These hGH domains can be fused to catalytic intein sequences, such that the Ic and In catalytic residues are brought in to proximity by addition of 5-chloro-2-trichloromethylimidazole, thereby inducing intein mediated splicing and peptide cyclization. Accordingly, these domains can be used for Prot1 and Prot2, as depicted in FIG. 10.

Finally, the SH2 domains are structural protein domains that recognize phosphorylated-tyrosine (P-Tyr) in a specific peptide context. The phosphorylation of tyrosine is a common event that follows the activation of tyrosine kinase in signal transduction pathways in response to a specific treatment of the cells. Thus, SH2 can be used, e.g., for Prot1 and the domain containing a particular Tyr, as Prot2.

1.5 Generation of Sterol-Conjugated Peptide Aptamers

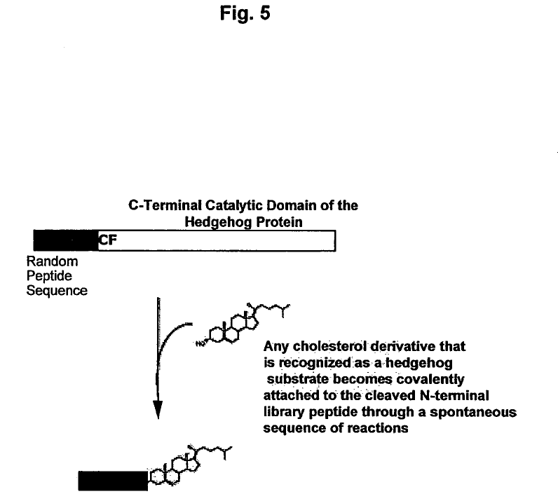

In another preferred embodiment, the invention also includes fusion moieties that can be attached to a peptide aptamer and are capable of conjugating the peptide aptamer to a ligand, e.g., a sterol. In a particular embodiment, the aptamer sequence is linked to a sufficient portion of the hedgehog polypeptide such that the aptamer is subsequently linked to a sterol, e.g., cholesterol, and targeted to a membrane (see, e.g., FIGS. 5, 11, and 24)

As described in detail herein, the peptide aptamer libraries can be generic (i.e., encode only minimal, random peptide aptamers), incorporate structural features for stability (e.g., the peptides aptamers are fused to scaffold proteins or produced as cyclic peptides) and/or detection (e.g., fused to GFP), or incorporate features that suit them for the study of particular phenotypes associated with certain processes or specific intracellular locations (e.g., the aptamer is fused to a heterologous localization domain).

1.6 Generation of Peptide Aptamers “Conotides” Derived from Conotoxins

Peptide aptamers (and libraries thereof) can also be generated using sequence motifs derived from conotoxins to produce peptide aptamers (referred to herein as “conotides”) which have desired structural and biologic properties (Jones et al, Composition and Therapeutic Utility of Conotoxins from Genus Conus. Patent Status 1996-2000. Exp. Opin. Ther. Patents 11: 603-623 (2001)). Conotoxins were originally isolated from predatory cone snails (genus Conus) which can immobilize and kill their prey using a cocktail of neurotoxic peptides referred to as conotoxins. These conotoxins are small, 10-30 amino acids in length, and they are constrained, usually by two or more sets of cysteine-mediated disulfide bonds. Naturally occurring conotoxins function by inhibiting the function of various ion channels present in neuronal or muscular tissues.

The present invention uses in vivo and synthetic combinatorial peptide chemistry to exploit the use of conotoxin backbones, to identify novel conotoxin or conotoxin-like peptides (conotides) with improved or altered target affinity or specificity. In addition, the invention also provides a variety of novel strategies that can be used alone or in combination to generate and screen peptide libraries in which certain conotoxin-like structural elements are retained, but which improve various characteristics of the conotoxin peptides, including but not limited to, increasing peptide stability, or removing the need for one or more of the disulfide bonds present in the naturally occurring prototypic backbone structures.

In accordance with the present invention, one or more cysteine residues is removed and the conotides generated are preferably highly stable and constrained. Cysteine independent constraints, e.g., linking moieties, featured in this type of molecule are designed to be less susceptible to the redox environment within the cell, thereby maintaining their structural integrity in various cellular microenvironments (e.g. endoplasmic reticulum, Golgi apparatus, lysosomes), or to degradation or redox effects in the extracellular environment or bloodstream of an animal or human. Moreover, synthesis of such random peptide libraries within the cell allows examination of their effects upon intracellular targets. This is in contrast to naturally occurring conotoxins, which evolved to act upon extracellular targets or ligands.

The constrained structural features and characteristics of the conotoxin peptides represent an ideal scaffolding strategy for the design and synthesis of peptide aptamer libraries. Indeed, the processes of natural selection that have led to their existence and prevalence in nature suggest that these are optimized formats for the generation of biologically active peptides, particularly peptides that act upon ionic or voltage gated channels. Due to their specificity, the effectiveness of small helical shapes, and the availability of a cysteine framework, conotoxin-based conotides are suitable for use as the basis for drugs with importance in a number of therapeutic areas.

The known, naturally occurring conotoxins have been divided into families based on conserved cysteine residues and function. A summary of the various conotoxins, their classification, structure, and mode of action is shown in Table 1, below. The several major superfamilies include the A, M, S, and O superfamilies; at least 4 other superfamilies may exist. A given conotoxin is usually highly specific for its target. Some examples include the α-conotoxins, which inhibit nicotinic acetylcholine receptors; ω-conotoxins, which block voltage-gated calcium channels; and δ-conotoxins, which alter activation of voltage-gated sodium channels

TABLE 1
Summary of the various conotoxins, their classification, structure, and mode of
action.

| Super | | | Linkage Pattern in Native |
|---|---|---|---|
| Family | Family | Mode of Action | Conotoxin |
| A | αβ | a Competitive antagonist of nAchRs Antagonist of α₁-adenoreceptors | 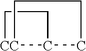 |
| | αA | Competitive antagonist of nAchRs | 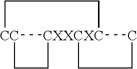 |
| | κA | Inhibits conductance of voltage- gated K⁺ channels | 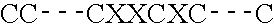 |
| M | μψ | Blocks Na⁺ channels by binding to Site I Non-competitive inhibitor of nAchRs | 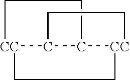 |
| O | δωγμO κBromosleeper Conotoxin GS | Delays Na⁺ channel inactivation Blocks Ca⁺² channels Modulates channels Blocks Na⁺ channels but does not bind to Site I Blocks K⁺ channels Unknown Blocks Na⁺ channels | 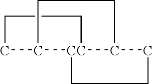 |
| S | σ | Competitive antagonist of the 5- HT₃ Receptor | 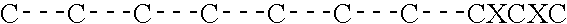 |
| T | τ | Unknown | 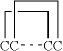 |
| | χ | Noradrenaline transporter blocker | 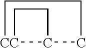 |
| P | N/A | Unknown | 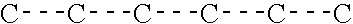 |
| Non- disulfide rich | Conantokin Contulakin Contryphan | Antagonist at NMDA receptor Agonist at neurotensin receptor Unknown | linear linear 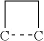 |

In Table 1, C represents cysteine residues, X represents a single amino acid residue, the dotted line represent a polypeptide linkage and the solid line represents a disulfide covalent linkage.

The A superfamily includes the α and αA families, which target nicotinic acetylcholine receptors, the ρ family, which is an antagonist of α1-adreno receptors, and the κA family, which inhibits conductance of voltage-gated K⁺ channels. The M superfamily includes the μ family of conotoxins, which block Na⁺ channels, as well as the ψ family, which noncompetitively inhibit nicotinic acetylcholine receptors.

The O superfamily consists of a number of families with an identical cysteine pattern. Members of the O superfamily include the δ, ψ, γ, μO, and κ families. These families can be used to block or deactivate ionic channels, including Na⁺, Ca²⁺, and K⁺ channels. The O superfamily also includes the conotoxin-GS family, which can be used to block Na⁺ channels, and this family also includes Bromosleeper.

There are several other families of conotoxins. Three other currently known superfamilies are S, T, and P. These superfamilies include the σ, τ, and χ. There are also several conotoxin-like peptides; these include conantokins, contulakin, and contryphan. Members of the conotoxin-related conantokin peptides function similarly to conotoxins, but do not contain disulfide bonds. Instead, the conantokin structure is dependent on the presence of calcium binding by numerous gamma-carboxy-glutamic acid residues within these peptides (Prorok et al., Biochemistry, 35(51):16528-34 (1996)). Contryphan contains only one disulfide bond. The activity of these conotoxin-like peptides requires the constrained helical shape present in the conotoxin/conantokin family and the disulfide bonds that sponsor and stabilize this helical configuration.

One exemplary conotoxin isolated and characterized was α-conotoxin GI. The 13 amino acid α-conotoxin GI peptide (ECCNPACGRHYSC-NH₂) has a pair of disulfide bonds. The intramolecular disulfide bond between Cys3 and Cys13 serves as a “clamp”, to keep the N- and C-terminal ends of the peptide in proximity. The N-terminal glutamic acid residue of GI can be deleted without affecting biological activity. The disulfide bond between Cys2 and Cys7 establishes the conserved small-loop structure characteristic of all α-conotoxins. The importance of this structure is evidenced by the observations that mutations in this small-loop region lead to a loss of biological activity, and substitution of Pro5 eliminates the biological activity of α-conotoxins MI and GI.

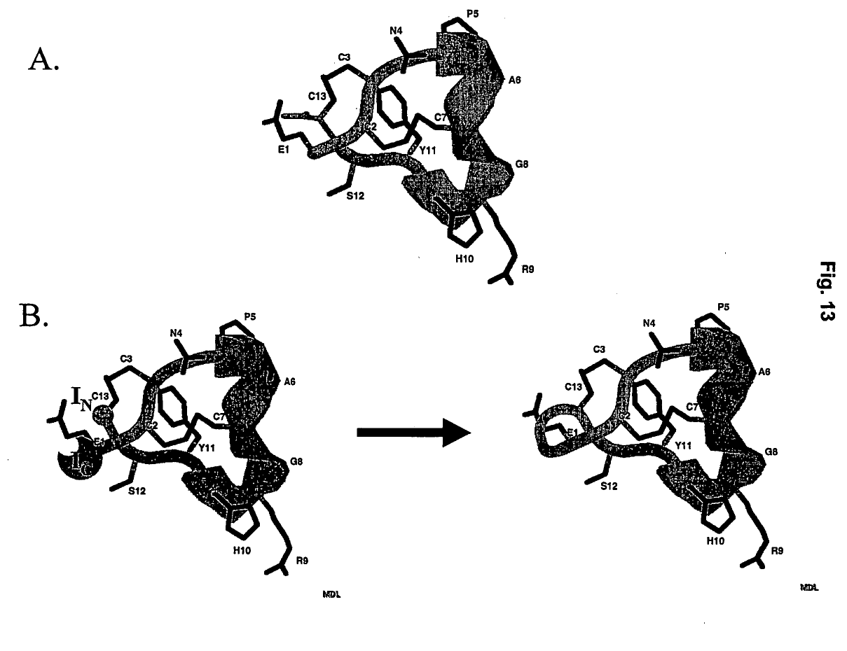

It is understood that any of the conotides described herein can, in lieu of or in addition to any actual or potentially linkages, can be cyclized using intein-mediated protein splicing (see, e.g., FIG. 13), for example, as indicated below in Table 2. Here, the left panel shows the cysteine positioning and spacing associated with the various subtypes of naturally occurring conotoxins, along with the configuration of structurally essential cysteine-mediated intramolecular disulfide bonds that are known to occur in these peptides. The right panel exemplifies the manner in which these respective subtypes can be equivalently integrated into an intein-splicing format and expressed in cells for the generation of conotides.

TABLE 2
Exemplary Conotides

| Canonical Conotoxin Sequences | I_{C}-Conotide-I_{N} |
|---|---|
| 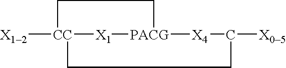 | 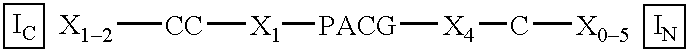 |
| 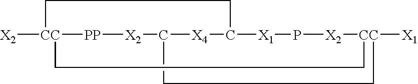 | 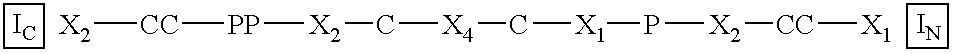 |
| 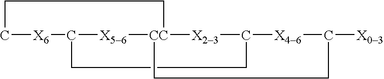 | 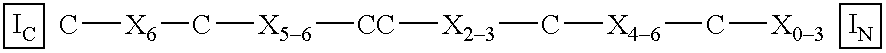 |

1.7 Generation of Synthetic Conotides

This invention also describes synthetic chemical approaches to the improvement of conotoxins or conotoxin derived peptides. The linking moieties of the conotides of the invention generally replace one or more of the disulfide bridges of the native conotoxins, respectively. Linking moieties can also be introduced at positions where a linkage would not inhibit the conotide from performing its intended function. Advantageously, the linking moieties enhance the stability of the aptamer in in vivo environments.

Conotides linked through polypeptide linking moieties may be synthesized in vivo through intein mediated cyclization chemistry described above. Alternatively, a variety of chemical ligation or linking methods, such as those described below, may also be used. Examples of linking moieties, include, for example, moieties which comprise disulfide bonds (1) and moieties which comprise thioether linkages (2). Disulfide bonds and thioether bonds are found in some naturally occurring peptides such as conotoxins and antimicrobial tachyplesins, protegrins and defensins are shown in the formulae below.

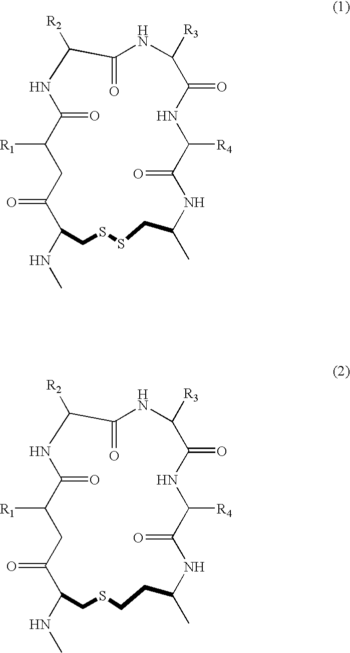

The linking moiety may also comprises carbon atoms in the atom chain. For example, the linking moiety may be alkyl, alkenyl, or alkynyl. Alkenyl linking moieties may be synthesized using ring-closing-metathesis (RCM) reactions. This reaction has been exploited for the synthesis of cyclic aptamers with alkenyl linking moieties (3, 4). The RCM reaction can be performed either in solution or on a solid support giving the cyclic peptide in a yield of 50-65%.

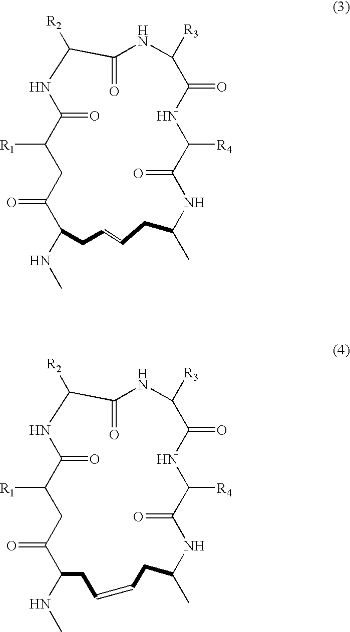

Other linking moieties also include those which link the N- and C-termini of the polypeptide aptamers of the invention together. End-to-end cyclic peptides are comparatively more stable to enzymatic digestion and have a better bioavailability due to the lack of N- and C-termini. Examples of linking moieties which can be used to link the ends of an aptamer include those shown in formulae 5, 6, 7, and 8. Other linking moieties which may be used include linking moieties which comprise one or more peptide bonds.

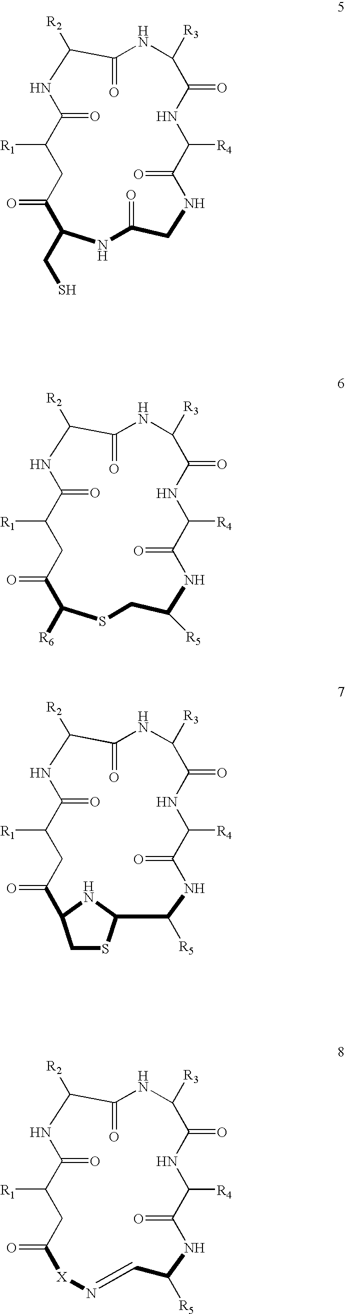

Conotides with peptidic linking moieties (such as those shown in 5) can be synthesized using an intramolecular thioester ligation reaction using art recognized techniques. For example, the unprotected N-Cys peptide thioester precursor undergoes a two-step reaction in 6 M Gua.HCl at pH 7.6 to afford the conotide in a quantitative yield [24]. Cyclic aptamers of type 6 can be obtained by a cascade thioester ligation.

Other linking moieties also include moieties such as the thiozolidine linking moiety shown in 7. The key intermediate for the synthesis of this compound is the unprotected peptide aldehyde. The unprotected peptide aldehyde can also be used for the synthesis of the cyclic aptamer 8 through an oxime ligation.

Conotides also can be formed by reacting at least two attaching moieties of linking residues with each other under appropriate conditions, such that the linking moiety is synthesized such as shown in structures 9-17 below. For example, in the formation of certain linking moieties the appropriate conditions comprise one or more linking reagents.

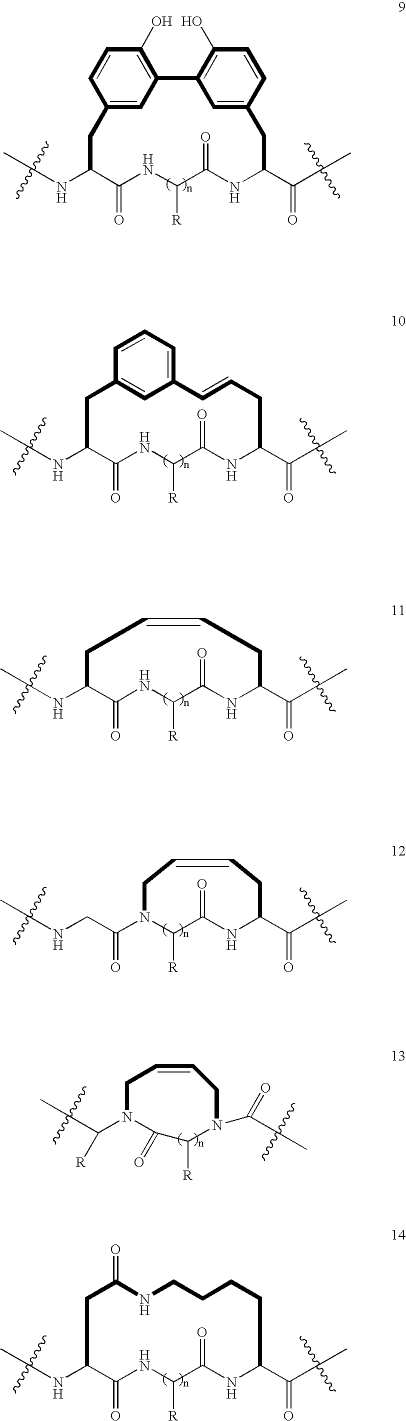

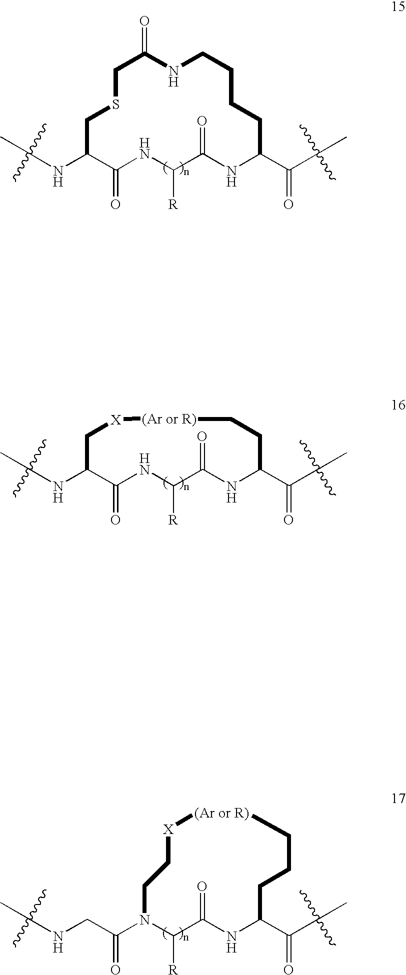

Linking reagents, such as sulfonyl chloride, dimethyl dichlorosilane, diisopropylamino, chlorophosphoramidite and metals (such as boron and titanium) are capable of temporary binding as a linking moiety. One of ordinary skill in the art will realize that other linking reagents can be employed to generate mixed carbonates, disulfides, ureas, acetals, ortho esters, phosphates and oxides as linking moieties.

The linking moiety may be synthesized at any amino acid residue site which includes a covalent attaching moiety, e.g., functionality capable of reacting with another covalent attaching moiety, optionally in the presence of a linking reagent, to form a linking moiety. Preferably, the attaching moiety comprises a hydroxyl, amino, or thiol group. Examples of attaching moieties include hydrogen, protecting groups, alkenes, alkynes, amines, carboxylicacids, halogenated aromatics, aldehydes and vinyl halide.

In one embodiment, the linking moiety comprises a silicon atom. In this embodiment, the linking residues (“Z”) are linked by a linking reagent. In a further embodiment, the linking residues each have a heteroatom-containing attaching moieties and are linked together by means of a silicon linking reagent such as dimethyldichlorosilane. In another embodiment of the invention, the linking residues each contain a hydroxyl or amine attaching moiety and diisopropylamino chlorophosphoramidite is used as the linking reagent.

In one embodiment, the linking residues each comprise alkene attaching moieties. These linking residues are then subjected to conditions appropriate for an intramolecular metathesis reaction, such that an alkene linking moiety is generated. Examples of suitable linking reagents include, but are not limited to transition metal catalysts (e.g., Grubbs' catalyst and others cited in Tet. Lett., 1999,40., 2247).

Transition metal mediated crosscoupling reactions may also be used to synthesize linking moieties. Examples of transition metal mediated cross coupling reactions which can be used include but are not limited to, palladium catalyzed Heck Reactions (e.g., in which a haloaromatic linking residue is reacted with a second linking residue under palladium (0) catalysis to yield aromatic and alkenyl linking moieties).

In an embodiment, the invention pertains to conotides of the formula (1):

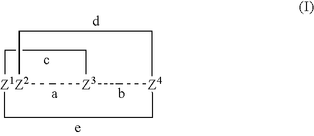

wherein
Z¹, Z², Z³, and Z⁴ are each independently selected linking residues;
a and b are each polypeptide linkers comprising one or more amino acid residues; and
c, d and e are each independently selected linking moieties or absent, provided that if each of Z¹, Z², Z³ and Z⁴ are cysteine, then at least one of c, d, and e is not a disulfide linking moiety; and provided that at least one of c, d, and e is not absent.

In a further embodiment, conotides of formula (I) comprise one or more linking moieties which are covalent linkages (e.g., c, d, or e), as described above. In a further embodiment, at least one of c, d, or e is a disulfide covalent linkage and at least two of Z¹, Z², Z³, and Z⁴ are cysteine. In another embodiment, at least one of c, d, and e is not a disulfide linkage. In certain embodiments, at least one of c, d, or e is a linking moiety with a hydrophobic linkage. For this embodiment, at least two of Z¹, Z², Z³, and Z⁴ comprise an aryl group. In a further embodiment, at least one of Z¹, Z², Z³ and Zare tryptophans. Examples of e include a polypeptide linkage (e.g., comprises at least a peptide bond). Other examples of linking moieties which may be used for one or more of c, d, or e, include ionic linkages. Preferably, the conotide of formula (I) is more stable to the redox environment in vivo, than a conotide with disulfide bonds at c and d and with e absent. In a further embodiment, a and b comprise between zero and twenty independently selected amino acid residues. In particular embodiments, a and b each comprise from about one to about ten amino acid residues. The number of amino acid residues present in a and b polypeptide linkers may vary, although the number of amino acid residues is selected such that the conotide is capable of performing its intended function.

Examples of conotides of the invention of formula (I), include, but are not limited to, conotides of the formulae:

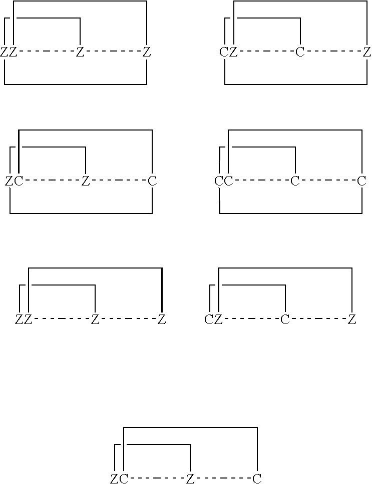

wherein each occurrence of Z is an independently selected linking residue other than cysteine, C is a cysteine residue, the solid lines are covalent linkages and the dotted lines represent polypeptide linkages.

The invention also pertains to conotides of the formula (II):

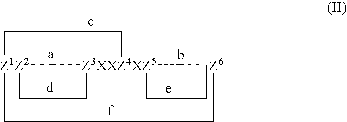

wherein
Z¹, Z², Z³, Z⁴, Z⁵, and Z⁶ are each independently selected linking residues;
X is an independently selected amino acid residue for each occurrence;
a and b are each independently selected polypeptide linker moieties comprising one or more amino acid residues; and
c, d, e, and f are each independently selected linking moieties or absent, provided that if each of Z¹, Z², Z³, Z⁴, Z⁵ and Z⁶ are cysteine, then at least one of c, d, e, and f is not a disulfide liking moiety; and provided that at least one of c, d, e, or f is not absent.

In a further embodiment, conotides of formula (II) comprise one or more linking moieties which are covalent linkages (e.g., c, d, e, or f), as described above. In a further embodiment, at least one of c, d, e, or f is a disulfide covalent linkage and at least two of Z¹, Z², Z³, Z⁴, Z⁵, and Z⁶ are cysteine. In another embodiment, at least one of c, d, e, and f is not a disulfide linkage. In certain embodiments, at least one of c, d, e, and f is a linking moiety with a hydrophobic linkage. For this embodiment, at least two of Z¹, Z², Z³, Z⁴, Z⁵, and Z⁶ comprise an aryl group. In a further embodiment, at least one of Z¹, Z², Z³, Z⁴, Z⁵ and Z⁶ are tryptophans. Examples of f include a polypeptide linkage (e.g., comprises at least a peptide bond). Other examples of linking moieties which may be used for one or more of c, d, e, or f, include ionic linkages. Preferably, the conotide of formula (II) is more stable to the redox environment in vivo, than a conotide with disulfide bonds at c, d, and e and with f absent. In a further embodiment, a and b comprise between zero and twenty independently selected amino acid residues. In particular embodiments, a and b each comprise from about one to about ten amino acid residues. The number of amino acid residues present in a and b polypeptide linkers may vary, although the number of amino acid residues is selected such that the conotide is capable of performing its intended function.

Examples of conotides of the invention of formula (II), include, but are not limited to, conotides of the formulae:

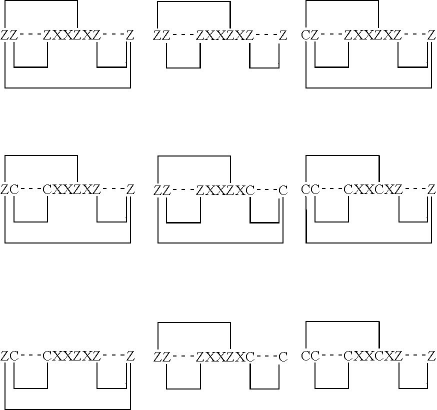

wherein each occurrence of Z is an independently selected linking residue other than cysteine, C is a cysteine residue, the solid lines are covalent linkages and the dotted lines represent polypeptide linkages.

In another embodiment, the invention pertains to conotides of formula (III): 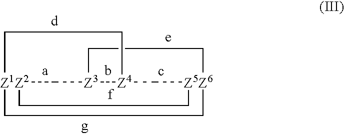
wherein
Z¹, Z², Z³, Z⁴, Z⁵, and Z⁶ are each independently selected linking residues;
a, b, and c are each independently selected polypeptide linkers comprising one or more amino acid residues; and
d, e, f, and g are each independently selected linking moieties or absent; provided that if each of Z¹, Z², Z³, Z⁴, Z⁵ and Z⁶ are cysteine, then at least one of e, d, f, and g is not a disulfide linking moiety; and provided that at least one of d, e, f, or g is not absent.

In a further embodiment, conotides of formula (III) comprise one or more linking moieties which are covalent linkages (e.g., d, e, f, or g), as described above. In a further embodiment, at least one of d, e, f, or g is a disulfide covalent linkage and at least two of Z¹, Z², Z³, Z⁴, Z⁵, and Z⁶ are cysteine. In another embodiment, at least one of d, e, f, and g is not a disulfide linkage. In certain embodiments, at least one of d, e, f, and g is a linking moiety with a hydrophobic linkage. For this embodiment, at least two of Z¹, Z², Z³, Z⁴, Z⁵, and Z⁶ comprise an aryl group. In a further embodiment, at least one of Z¹, Z², Z³, Z⁴, Z⁵ and Z⁶ are tryptophans. Examples of g include a polypeptide linkage (e.g., comprises at least a peptide bond). Other examples of linking moieties which may be used for one or more of d, e, f, or g include ionic linkages. Preferably, the conotide of formula (III) is more stable to the redox environment in vivo, than a conotide with disulfide bonds at d, e, and f and with g absent. In a further embodiment, a, b, and c comprise between zero and twenty independently selected amino acid residues. In particular embodiments, a, b, and c each comprise from about one to about ten amino acid residues. The number of amino acid residues present in a, b and c polypeptide linkers may vary, although the number of amino acid residues is selected such that the conotide is capable of performing its intended function.

Examples of conotides of the invention of formula (III), include, but are not limited to, conotides of the formulae:

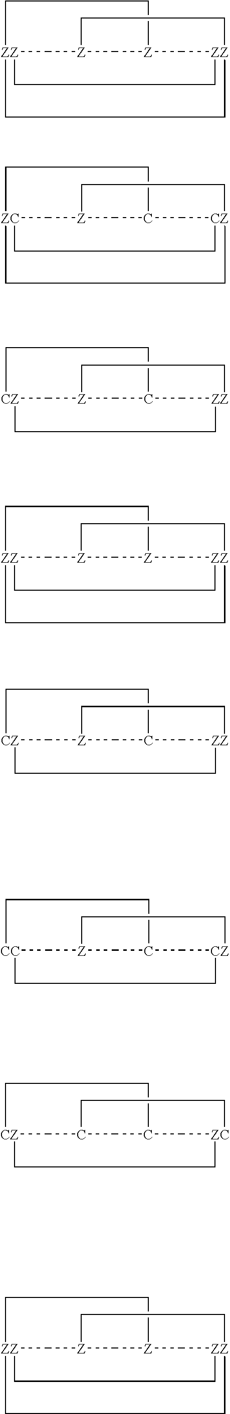

wherein each occurrence of Z is an independently selected linking residue other than cysteine, C is a cysteine residue, the solid lines are covalent linkages and the dotted lines represent polypeptide linkages.

The invention also pertains to conotides of the formula (IV): 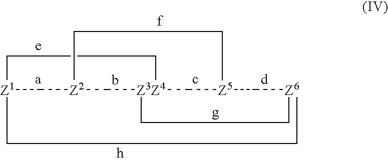
wherein
Z¹, Z², Z³, Z⁴, Z⁵, and Z⁶ are each independently selected linking residues;
a, b, c, and d are each independently selected polypeptide linkers comprising one or more amino acid residues; and
e, f, g, and h are each independently selected linking moieties or absent; and, provided that if each of Z¹, Z², Z³, Z⁴, Z⁵ and Z⁶ are each cysteine, then at least one of e, f, g, and h is not a disulfide linking moiety; and provided that at least one of e, f, g, or h is not absent.

In a further embodiment, conotides of formula (IV) comprise one or more linking moieties which are covalent linkages (e.g., e, f, g, or h), as described above. In a further embodiment, at least one of e, f, g, or h is a disulfide covalent linkage and at least two of Z¹, Z², Z³, Z⁴, Z⁵, and Z⁶ are cysteine. In another embodiment, at least one of e, f, g and h is not a disulfide linkage. In certain embodiments, at least one of e, f, g, and h is a linking moiety with a hydrophobic linkage. For this embodiment, at least two of Z¹, Z², Z³, Z⁴, Z⁵, and Z⁶ comprise an aryl group. In a further embodiment, at least one of Z¹, Z², Z³, Z⁴, Z⁵ and Z⁶ are tryptophans. Examples of h include a polypeptide linkage (e.g., comprises at least a peptide bond). Other examples of linking moieties which may be used for one or more of e, f, g, and h include ionic linkages. Preferably, the conotide of formula (IV) is more stable to the redox environment in vivo, than a conotide with disulfide bonds at e, f and g and with h absent. In a further embodiment, a, b, c, and d comprise between zero and twenty independently selected amino acid residues. In particular embodiments, a, b, c, and d each comprise from about one to about ten amino acid residues. The number of amino acid residues present in a, b, c, and d polypeptide linkers may vary, although the number of amino acid residues is selected such that the conotide is capable of performing its intended function.

Examples of conotides of the invention of formula (IV), include, but are not limited to, conotides of the formulae:

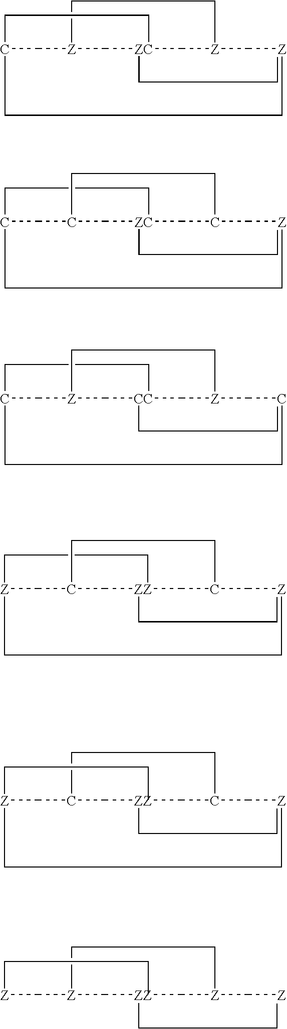

wherein each occurrence of Z is an independently selected linking residue other than cysteine, C is a cysteine residue, the solid lines are covalent linkages and the dotted lines represent polypeptide linkages.

In yet another embodiment, the invention pertains to conotides of the formula (V):

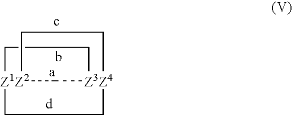

wherein
Z¹, Z², Z³, and Z⁴ are each independently selected linking residues;
a is a polypeptide linker comprising one or more amino acid residues; and
b, c and d are each independently selected linking moieties or absent, provided that if each of Z¹, Z², Z³ and Z⁴ are cysteine, then at least one of b, c, and d is not a disulfide linking moiety; and provided that at least one of b, c, and d is not absent.

In a further embodiment, conotides of formula (V) comprise one or more linking moieties which are covalent linkages (e.g., b, c, or d), as described above. In a further embodiment, at least one of b, c, or d, is a disulfide covalent linkage and at least two of Z¹, Z², Z³, and Z⁴ are cysteine. In another embodiment, at least one of b, c, and d is not a disulfide linkage. In certain embodiments, at least one of b, c, or d is a linking moiety with a hydrophobic linkage. For this embodiment, at least two of Z¹, Z², Z³, and Z⁴ comprise an aryl group. In a further embodiment, at least one of Z¹, Z², Z³ and Z⁴ are tryptophans. Examples of d include a polypeptide linkage (e.g., comprises at least a peptide bond). Other examples of linking moieties which may be used for one or more of b, c, or d , include ionic linkages. Preferably, the conotide of formula (V) is more stable to the redox environment in vivo, than a conotide with disulfide bonds at b and c and d absent. In a further embodiment, a comprises between zero and twenty independently selected amino acid residues. In particular embodiments, a comprises from about one to about ten amino acid residues. The number of amino acid residues present in a polypeptide linkers may vary, however, generally the number of amino acid residues is selected such that the conotide is capable of performing its intended function.

Examples of conotides of the invention of formula (V), include, but are not limited to, conotides of the formulae:

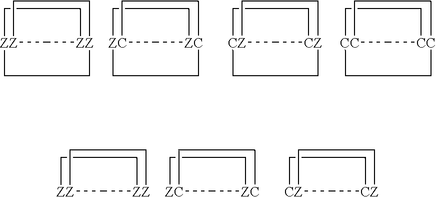

wherein each occurrence of Z is an independently selected linking residue other than cysteine, C is a cysteine residue, the solid lines are covalent linkages and the dotted lines represent polypeptide linkages.

In a further embodiment, the invention pertains to conotides of the formula (VI):

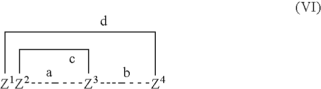

wherein
Z¹, Z², Z³, and Z⁴ are each independently linking residues;
a and b are polypeptide linkers comprising one or more amino acid residues; and
c and d are each independently selected linking moieties or absent, provided that if each of Z¹, Z², Z³ and Z⁴ are cysteine, then at least one of c and d is not a disulfide linking moiety; and provided that at least one of c and d is not absent.

In a further embodiment, conotides of formula (VI) comprise one or more linking moieties which are covalent linkages (e.g., c or d) as described above. In a further embodiment, at least one of c or d is a disulfide covalent linkage and at least two of Z¹, Z², Z³, and Z⁴ are cysteine. In another embodiment, at least one of c, and d is not a disulfide linkage. In certain embodiments, at least one of c or d is a linking moiety with a hydrophobic linkage. For this embodiment, at least two of Z¹, Z², Z⁴, and Z⁴ comprise an aryl group. In a further embodiment, at least one of Z¹, Z², Z³ and Z⁴ are tryptophans. Examples of d include a polypeptide linkage (e.g., comprises at least a peptide bond). Other examples of linking moieties which may be used for one or more of c or d include ionic linkages. Preferably, the conotide of formula (I) is more stable to the redox environment in vivo, than a conotide with disulfide bonds at c and d. In a further embodiment, a and b comprise between zero and twenty independently selected amino acid residues. In particular embodiments, a and b each comprise from about one to about ten amino acid residues. The number of amino acid residues present in a and b polypeptide linkers may vary, however, generally the number of amino acid residues is selected such that the conotide is capable of performing its intended function.

Examples of conotides of the invention of formula (VI), include, but are not limited to, conotides of the formulae:

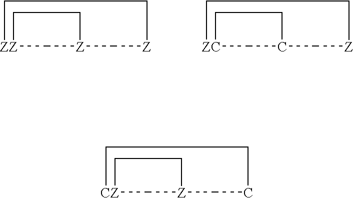

wherein each occurrence of Z is an independently selected linking residue other than cysteine, C is a cysteine residue, the solid lines are covalent linkages and the dotted lines represent polypeptide linkages.

In one embodiment, the invention pertains to conotides of the formula (VII):

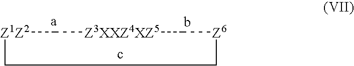

wherein:
Z¹, Z², Z³, Z⁴, Z⁵, and Z⁶ are each independently selected linking residues;
X is an independently selected amino acid residue for each occurrence;
a and b are each independently selected polypeptide linkers comprising one or more amino acid residues; and
c is a linking moiety or absent, provided that if each of Z¹, Z², Z³, Z⁴, Z⁵, and Z⁶ are cysteine, then c is not absent.

In a further embodiment, conotides of formula (VII) comprise a covalent linkage (e.g., c), as described above. In a further embodiment, c is a disulfide covalent linkage and Z¹ and Z⁶ are each cysteine. In another embodiment, c is not a disulfide linkage. In certain embodiments, c is a linking moiety with a hydrophobic linkage. For this embodiment, Z¹ and Z⁶ may comprise an aryl group. In a further embodiment, Z¹ and Z⁶ are tryptophans. The linkage c also may be a polypeptide linkage (e.g., comprises at least a peptide bond). The linkage c also may be an ionic linkage. Preferably, the conotide of formula (VII) is more stable to the redox environment in vivo, than a conotide without the linkage at c. In a further embodiment, a and b comprise between zero and twenty independently selected amino acid residues. In particular embodiments, a and b each comprise from about one to about ten amino acid residues. The number of amino acid residues present in a and b polypeptide linkers may vary, although the number of amino acid residues is selected such that the conotide is capable of performing its intended function.

The invention also pertains, at least in part, to conotides of the formula (VIII):

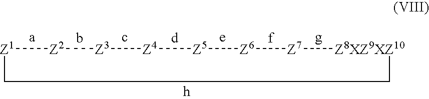

wherein
Z¹, Z², Z³, Z⁴, Z⁵, Z⁶, Z⁷, Z⁸, Z⁹, and Z¹⁰ are each independently selected linking residues;
X is an independently selected amino acid residue for each occurrence;
a, b, c, d, e, f, and g are each independently selected polypeptide linkers comprising one or more amino acid residues; and
h is a linking moiety or absent, provided that if each of Z¹, Z², Z³, Z⁴, Z⁵, Z⁶, Z⁷, Z⁸, Z⁹, and Z¹⁰ are cysteine, then h is not absent.

In a further embodiment, conotides of formula (VIII) comprise a covalent linkage (e.g., h), as described above. In a further embodiment, h is a disulfide covalent linkage and Z¹ and Z¹⁰ are each cysteine. In another embodiment, h is not a disulfide linkage. In certain embodiments, h is a linking moiety with a hydrophobic linkage. For this embodiment, Z¹ and Z¹⁰ may comprise an aryl group. In a further embodiment, Z¹ and Z¹⁰ are tryptophans. The linkage h also may be a polypeptide linkage (e.g., comprises at least a peptide bond). The linkage h also may be an ionic linkage. Preferably, the conotide of formula (VIII) is more stable to the redox environment in vivo, than a conotide without the linkage at h. In a further embodiment, a, b, c, d, e, f, and g each comprise between zero and twenty independently selected amino acid residues. In particular embodiments, a, b, c, d, e, f, and g each comprise from about one to about ten amino acid residues. The number of amino acid residues present in a, b, c, d, e, f, and g polypeptide linkers may vary, although the number of amino acid residues is selected such that the conotide is capable of performing its intended function.

In yet another embodiment, the invention pertains to conotides of the formula (IX):

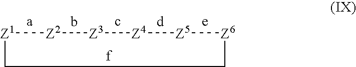

wherein
Z¹, Z², Z³, Z⁴, Z⁵, and Z⁶ are each independently selected linking residues;
a, b, c, d, and e are each independently selected polypeptide linkers comprising one or more amino acid residues; and
f is a linking moiety or absent, provided that if each of Z¹, Z², Z³, Z⁴, Z⁵, and Z⁶, are cysteine, then f is not absent.

In a further embodiment, conotides of formula (IX) comprise a covalent linkage (e.g., f), as described above. In a further embodiment, f is a disulfide covalent linkage and Z¹ and Z⁶ are each cysteine. In another embodiment, f is not a disulfide linkage. In certain embodiments, f is a linking moiety with a hydrophobic linkage. For this embodiment, Z¹ and Z⁶ may comprise an aryl group. In a further embodiment, Z¹ and Z⁶ are tryptophans. The linkage f also may be a polypeptide linkage (e.g., comprises at least a peptide bond). The linkage f also may be an ionic linkage. Preferably, the conotide of formula (IX) is more stable to the redox environment in vivo, than a conotide without the linkage at f. In a further embodiment, a, b, c, d, and e, each comprise between zero and twenty independently selected amino acid residues. In particular embodiments, a, b, c, d, and e each comprise from about one to about ten amino acid residues. The number of amino acid residues present in a, b, c, d, and e polypeptide linkers may vary, although the number of amino acid residues is selected such that the conotide is capable of performing its intended function.

1.8 Additional Chemical Modifications of Peptide Aptamers

In addition to the above described modifications of peptide aptamers using fusion moieties, cysteine replacement, and intramolecular linkages, it is also understood that the peptide aptamer of the invention can be further modified to alter a specific property of the peptide aptamer while retaining the ability of the aptamer to, e.g., bind to a ligand. For example, in one embodiment, the peptide aptamer is further modified to alter a pharmacokinetic property of the peptide aptamer, such as in vivo stability or half-life. In another embodiment, the peptide aptamer is further modified to label the peptide aptamer with a detectable substance. In yet another embodiment, the peptide aptamer is further modified to couple the peptide aptamer to an additional therapeutic moiety.

To further chemically modify the peptide aptamer, such as to alter the pharmacokinetic properties (e.g., stability or half-life) of the peptide aptamer, reactive groups can be derivatized. For example, linear peptide aptamers can be modified at the carboxy-terminal end to reduce the ability of the peptide aptamer to act as a substrate for carboxypeptidases. Examples of preferred C-terminal modifiers include an amide group (i.e., a peptide amide), an alkyl or aryl amide group (e.g., an ethylamide group or a phenethylamide group) a hydroxy group (i.e., a peptide alcohol) and various non-natural amino acids, such as D-amino acids and β-alanine. Alternatively, the amino-terminal end of the peptide aptamer can be further modified, for example, to reduce the ability of the peptide aptamer to act as a substrate for aminopeptidases.

A peptide aptamer of the invention can also be reacted and labeled with a detectable substance. Suitable detectable substances include various prosthetic groups, fluorescent materials, luminescent materials, and radioactive materials. Examples of suitable prosthetic groups include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹⁴C, ¹²³I, ¹²⁴I, ¹²⁵I, ¹³¹I, ⁹⁹Tc, ³⁵S or ³H. In a preferred embodiment, a peptide aptamer is radioactively labeled with ¹⁴C, either by incorporation of ¹⁴C into the modifying group or one or more amino acid structures in the peptide aptamer. Labeled aptamer compounds can be used to assess the in vivo pharmacokinetics of the peptide aptamer, as well as to detect a desired activity of the peptide aptamer, e.g., affinity, stability, bioavailability, for example, for diagnostic purposes. Such activities can be detected using a labeled peptide aptamer either in vivo or in an in vitro sample derived from a subject or art recognized animal model.

In an alternative chemical modification, a peptide aptamer of the invention is prepared in a “prodrug” form. For example, in this type of peptide aptamer, the modulating group can be present in a prodrug form that is capable of being converted upon metabolism into the form of an active modulating group. Such a prodrug form of a modifying group is referred to herein as a “secondary modifying group.” A variety of strategies are known in the art for preparing peptide prodrugs that limit metabolism in order to optimize delivery of the active form of the peptide-based drug (see e.g., Moss, J. (1995) in Peptide-Based Drug Design: Controlling Transport and Metabolism, Taylor, M. D. and Amidon, G. L. (eds), Chapter 18. Additionally strategies have been specifically tailored to achieving CNS delivery based on “sequential metabolism” (see e.g., Bodor, N., et al. (1992) Science 257:1698-1700; Prokai, L., et al. (1994) J. Am. Chem. Soc. 116:2643-2644; Bodor, N. and Prokai, L. (1995) in Peptide-Based Drug Design: Controlling Transport and Metabolism, Taylor, M. D. and Amidon, G. L. (eds), Chapter 14. In one embodiment of a prodrug form of a peptide aptamer of the invention, the modifying group comprises an alkyl ester to facilitate blood-brain barrier permeability. In another embodiment, the prodrug aptamer is in the form of an unspliced intein/aptamer fusion.

II. Gene Delivery and Gene Expression

2.1 Improved Gene Delivery Using Receptors to Increase Vector Uptake

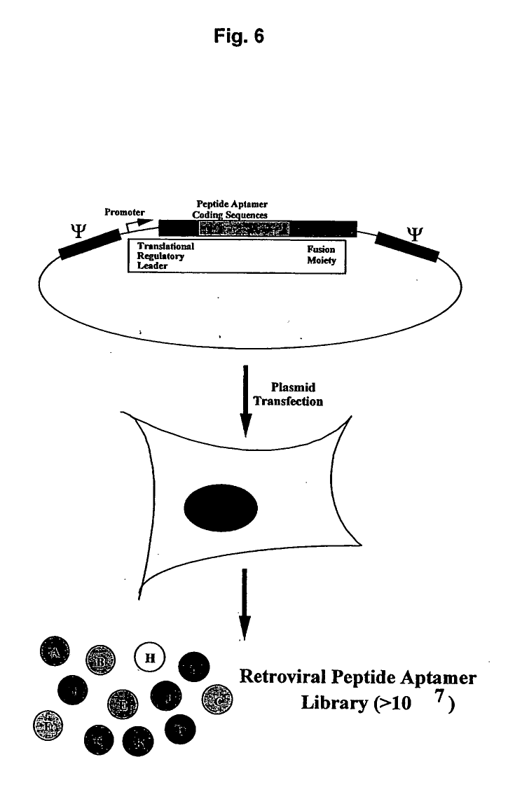

The above described peptide aptamers (and libraries thereof) can be generated using art recognized nucleic acid synthesis and vector systems suitable for expressing nucleic acid sequences in cells, e.g., prokaryotic or eukaryotic cells. Various vectors, as well as amphotropic and ecotropic packaging cell lines, can be used for production of high titers of retroviruses that infect eukaryotic cells, e.g., mouse or human cells. In a preferred embodiment, the invention employs high-titer retroviral packaging systems to produce peptide aptamer libraries (Burns et al., 1993; Pear et al., 1993) (see FIG. 6). These delivery and expression systems can be adapted for the efficient infection of a variety of cell types.

However, the efficient screening of retroviral peptide aptamer libraries requires that the percentage of cells in a population of cells undergoing transduction (gene uptake) be optimized. Like most other viruses, retroviruses use endogenous proteins associated with the cell membrane to mediate attachment and entry into the host mammalian cell. Proteins associated with the retroviral envelope interact specifically with host cell proteins, therefore the pattern of expression of a receptor associated with a given receptor is a major determinant of the animal species (or even specific tissues within a given species) that are susceptible to infection by a given retrovirus. Thus, the proteins associated with the envelope of a specific infectious retrovirus, combined with the identity of mammalian cells expressing the cognate receptor for such viral envelope proteins, determines the range of infectivity of a given retroviral particle.

Accordingly, the invention includes the efficient gene delivery of peptide aptamer libraries using libraries incorporated into a retrovirus where, either the retrovirus, recipient cell type, or both, are optimized for virus uptake. For example, retroviral particles can be packaged to present amphotropic receptors, which mediate viral transduction and entry into all types of mammalian cells, and even a variety of non-vertebrate cell types. In the case of ecotropic retroviruses, packaging is carried out in cell lines that produce virus containing the env protein of Moloney murine leukemia virus (MoMuLV). The MuLV receptor has been identified as a membrane spanning amino acid transporter protein known as ATRC-1 (Albritton et al., 1989). Only cells expressing the mouse form of ATRC-1 are susceptible to infection or transduction by ecotropic retroviruses or retroviral vectors. Thus, cells that are not normally susceptible to ecotropic infection by MuLV infection (e.g. Chinese hamster ovary cells, human cells) become infectable upon expression of the ATRC-1 receptor (Albritton et al., 1993). Moreover, as disclosed in this invention, mouse cells that are not naturally susceptible to efficient retroviral gene transduction using recombinant ecotropic retroviral vectors, are rendered highly susceptible upon stable over-expression of mouse ATRC-1.

The invention provides an additional use of the ATRC-1 receptor in the screening of retrovirus-encoded peptide aptamer libraries which addresses the safety of use and handling of retroviral libraries encoding random peptides. By definition, libraries encoding highly diverse random peptide libraries have the potential to produce peptides with a wide variety of biological functions. Moreover, in cells in which stable integration and expression of the retroviral genome occurs. the effects manifest by the encoded peptide aptamer are stable and irreversible. As such, a randomly derived retrovirus within a library could potentially encode a peptide with biological activity that by definition renders it potentially hazardous. For example, a peptide aptamer capable of immortalization or even transformation of mammalian cells would pose a limited but real biological threat to individuals handling it, were it able to infect human cells.

Accordingly, the invention provides for the use of retroviruses containing ecotropic receptors in the production and screening of peptide aptamer libraries. The invention further provides for the screening of such libraries in human (or other cell lines not normally susceptible to infection by ecotropic retroviruses) using cell lines that have been genetically altered to stably express the ATRC-1 ecotropic receptor. This approach offers the opportunity to screen and test retroviral aptamer libraries in mammalian cells, but using methods that minimize or eliminate potential risk to the experimenter.

2.2 Improved Gene Expression Using Chromatin Insulator Elements

Even with efficient gene delivery, optimal gene expression must be achieved in order to carry out an effective phenotypic screen of peptide aptamer libraries. It is known that, even though gene delivery using retroviral transduction generally results in stable integration and maintenance of the gene, expression per se occurs in only a subset of the recipient cells, and that expression frequently declines even in those cells that initially expressed the gene at high levels. For phenotypically screening peptide libraries that are transduced (delivered) into cells, it important to optimize the percentage of cells in which expression of the insert encoding the library aptamer occurs, and furthermore, under most conditions, to perpetuate that expression to the greatest extent possible.

This invention provides methods and compositions for using chromatin insulator elements for the improvement of retroviral vectors and their use in generating and screening retroviral libraries in mammalian cells.

The invention also encompasses the use of chromatin insulator elements to optimize the establishment of populations of cells expressing retroviral libraries early after transduction and proviral integration, as well as their utility in perpetuating expression throughout the growth of these cells, and the use of these cells in phenotypic selection assays either in tissue culture or in animals.

The chromatin insulator is a genetic element that is engineered into the retroviral library vector itself, and serves to augment and perpetuate expression of the retroviral library inserts at each site of integration within a plurality of cells, and throughout the continued growth or differentiation of the cells. This is of particular use in perpetuated expression in transgenic mouse models, or in experiential systems where expression in myeloid or lymphoid cell systems is desired.

III. Screening Methods

3.1 Screening Methods for Identifying Peptide Aptamers Capable of Altering a Cell Phenotype

The above peptide libraries and improved methods for gene delivery and expression can be used to carry out high efficient screens to identify peptide aptamers capable of altering a cell phenotype.

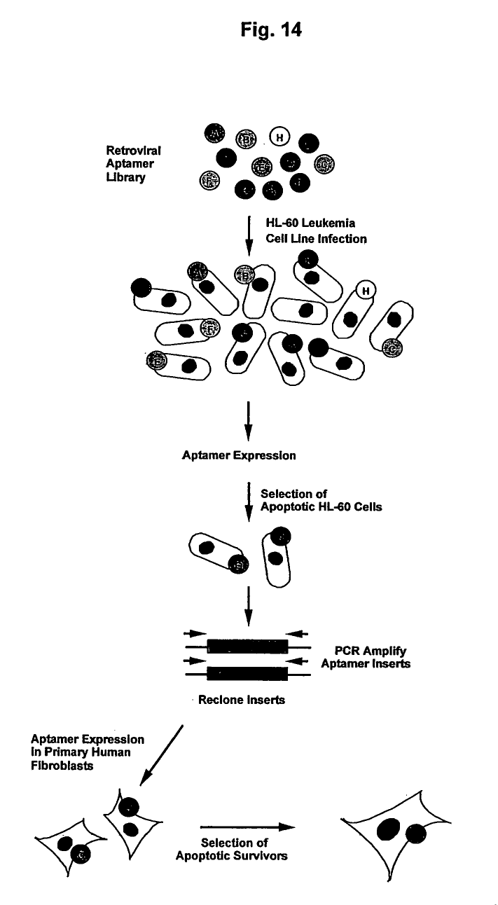

To carrying out a typical screening method of the invention, a population of cells, preferably a clonal population of eukaryotic cells is transfected or infected and cultured under appropriate conditions. Then, a cell or cells exhibiting a desired phenotype or a phenotype which differs from other cells in the cell population, is selected or isolated, for example, using FACS. Coding sequences of aptamers selected in the first round of screening can be amplified by PCR, re-cloned, and re-introduced into naïve cells. Phenotypic selection can then be repeated in order to validate individual aptamers within the original pool (e.g., as shown in FIG. 14). Aptamer coding sequences within cells identified in subsequent rounds of selection can be iteratively amplified and subcloned and the sequences of active aptamers can then be determined by DNA sequencing using standard techniques. This strategy can be applied to the identification of aptamers associated with a wide variety of cellular processes including, e.g., cell proliferation, regulation of apoptosis, protein trafficking or transport, cell motility or differentiation, metabolic state, and modulation of various signal transduction.

Peptide aptamer libraries of the present invention can have a complexity of up, e.g., up to ˜10⁸ or more allowing for the screening of a large numbers of aptamers (e.g.,) in a single experiment using any appropriate cell type (e.g., to test 10⁷ or preferably, 10⁹ combinations, 100 tissue culture vessels each containing 10 mls of media with 10⁶ cells/ml can be used). Thus, the present invention surpasses existing strategies that rely on target identification and selection, including those based on elucidation of specific protein-protein interactions, phenotypic gene expression profiling, or genotypic analysis. This is especially advantageous in the study of complex and highly diverse diseases, such as cancer and other diseases involving signal transduction pathways, as well as infectious diseases, such as viral diseases, where the virus has co-opted one or more cellular pathways (e.g., in order to replicate or avoid detection by the host immune system).

In a particular embodiment, the invention is used for the identification of aptamers that modulate measurable cellular processes (i.e., phenotypes). These include, but are not limited to: apoptosis, susceptibility to pathology associated with infection by a virus, bacterium, toxin, or prion, induction or repression of expression of one or more genes or gene products, differentiation, senescence, transport of intracellular molecules, including ions, nucleic acids, proteins, lipids, hormones, or metabolites, stability of any specified intracellular molecules, including ions, nucleic acids, proteins, lipids, or metabolites, or modulation of signalling by cellular receptors or their respective downstream signaling effectors. Other cellular processes may also be probed using the above approach, such as hormone or lipid metabolism, metabolism or homeostasis, and pathological conditions thereof.

In a specific embodiment, the cellular process of hair growth or hair loss, hair follicle differentiation, modification of hair follicle development, melanogenesis, modulation of hair shaft elongation, modulation of skin differentiation, or modulation of the hair growth cycle from telogen (resting stage) to anagen (growing stage) can be carried out using the peptides of the invention and any art recognized assay for measuring a change in one of the foregoing characteristics associated with hair growth or loss (see e.g., U.S. Pat. Nos. 5,767,152; 5,840,690; 5,527,772; and 5,407,944).

3.2 In Vitro Screening Methods for Identifying a Peptide Aptamer Capable of Killing a Cancer Cell

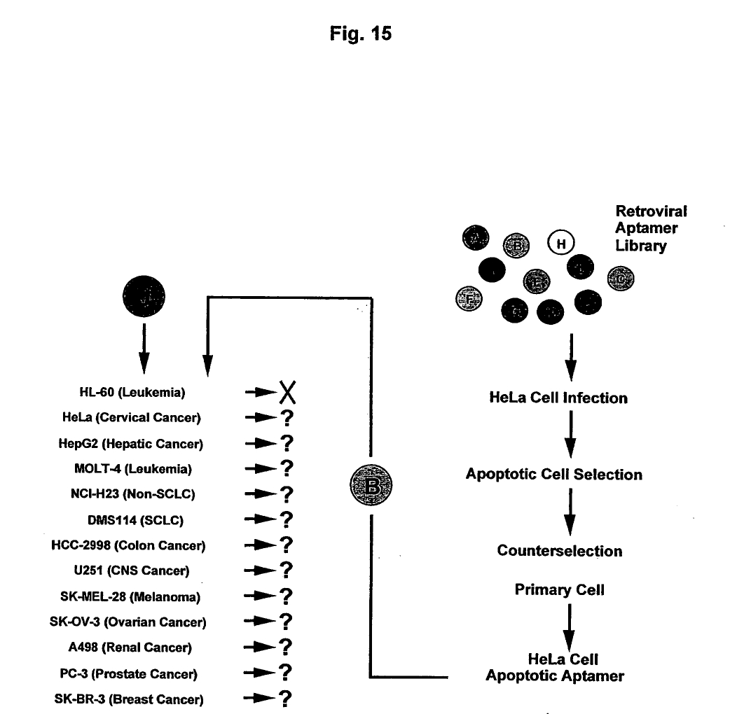

As indicated above, the screening methods of the invention can be adapted to identify aptamers that can alter any number of cellular phenotypes, including, e.g., the cancer phenotype. Accordingly, in one embodiment, a peptide aptamer library of the invention is tested for its ability to inhibit the growth, induce apoptosis, or kill a cancer cell, as compared to a normal cell. For example, the peptide aptamer can be tested in a clonal cancer cell line, and preferably, a panel of clonal cancer cell lines, such as those in the DTP Human Tumor Cell Line Screen which represents a diverse set of clonal cancer cells derived from various types of human tumors (Monks et al., 1991; see also FIG. 15). The susceptibility of each cancer cell type to exhibit a phenotypic change, for example inhibited growth or apoptosis when expressing the peptide aptamer, is then measured. This approach allows for determining the specificity of the bioactivity of the peptide aptamer across a range of different cancer types.

The library screening process can then be repeated in a second cell line in which expression of the first aptamer did not induce apoptosis. Aptamers identified in screening of this second cell line can also be tested against an entire cell line panel representative, e.g., of different cell types or cancers. This iterative process of identifying an aptamer with activity in one cell line, determining its activity against other cell lines, followed by identification of additional aptamers active against other cell lines, eventually leads to coverage of the entire cell line panel: a set of aptamers that induces apoptosis in at least one type of cancer cell line in the panel. An idealized compilation of the outcome of this process is shown in FIG. 15. It is important to reiterate that the pattern of susceptibility of a cancer cell to the identified set of aptamers can serve as a phenotype in and of itself, since this susceptibility can indicate the manifestation of the aggregate of changes that the cell underwent in its pathogenesis. Thus, this functional categorization of cancer cells can be extremely valuable, even in the absence of a full understanding of the molecular basis of the action of a given aptamer.

3.3 Screening Methods for Identifying Aptamers Having Antiviral Activity

The screening methods of the invention can also be used to identify peptide aptamers that have antiviral activity. In particular, the screening methods of the invention are suitable for identifying aptamers that fall into three classes: i) aptamers that modulate cellular processes that render the cell resistant to lysis or other pathological effects resulting from viral infection, but in which virus production by the cell can still occur; (ii) aptamers that inhibit or alter viral functions required for viral replication; and/or (iii) aptamers that inhibit a cellular function required for viral replication.

A typical screen for identifying an aptamer having antiviral activity in one of the above-mentioned classes is carried out by introducing the peptide aptamers into an appropriate cells as described herein that have either been previously infected, coinfected, or are later infected with a virus. Aptamers that alter the viral-mediated cell phenotypes and/or viral activity (e.g., viral replication, production) are determined using art recognized techniques (e.g., PCR, histological criteria, infectivity assays, or cell viability).

The screening methods of the invention are uniquely well suited for identifying aptamers that influence a phenotypic property associated with an infected cell that is not observed in an equivalent or comparable non-infected cell. For example, such a phenotypic property can be the expression of a specific cellular or viral antigen by the infected cell, or other functions that influence the pathogenesis or disease-causing functions mediated by infection of an animal by a given virus.

Accordingly, the first class of aptamers identified includes aptamers that alters any cellular function that, as a result of a virus-induced response, leads to cell death or pathogenesis. For example, induction of cellular PKR activity or transcriptional activation by NF-kappaB is associated with apoptosis of virally infected cells; thus attenuation of signals that manifest this response will render the infected host cell refractory to virus-induced death. Identifying peptide aptamers capable of modulating interferon response or other events associated with the intracellular signaling cascade response to viral infection is also within the scope of this invention.

The second class of aptamers identified includes aptamers that inhibit functions of virus encoded enzymes, as well as cell entry, aberrant localization of viral proteins, inhibition of viral packaging, or inhibition of virus release.

The third class of aptamers identified includes aptamers that effect cellular events necessary for the virus life cycle.

It is also understood that the above screening methods of the invention can also be applied to identifying aptamers that alter the activity of pathogens that have certain viral-like properties but are not a virus per se, for example, a prion.

3.4 In Vivo Screening Methods for Identifying Peptide Aptamers Capable of Altering a Cell Phenotype Using Transgenic Animals

The screening methods of the invention can also be used to identify peptide aptamers capable of altering a cell phenotype in a non-human transgenic or gene-knockout animal. For example, in one embodiment, a library of peptide aptamers encoded in an eukaryotic expression vector, e.g., a retroviral vector can be introduced into a transgenic animal having a detectable phenotype. The detectable phenotype may be a visually or molecularly recognizable occurrence and includes, for example, an alteration in the growth, maintenance, migration, or function of a cell type or tissue of the animal.

Transgenic animals suitable for introducing an aptamer library include animals engineered to have, e.g., a cancer (e.g., an animal having a constitutive promoter driving the expression of an oncogene or, alternatively, an animal engineered to lack a tumor suppressor) thereby allowing for the screening of aptamers which can abrogate a cancer phenotype. Alternatively, animals engineered to have a gene disruption, i.e., a transgenic “knock-out” animal, can be used to screen the aptamer library for peptide aptamers that can rescue the function normally provided by the disrupted gene. Using either of the foregoing strategies, peptide aptamers that can affect a “gain of function” or “loss of function” can be screened or selected for in vivo. Then, the cells or tissue from an animal exhibiting the desired phenotype are then used as a source of biological material for the isolation and identification of the nucleic acid encoding the peptide aptamer associated with the phenotype using art recognized techniques.

The invention also encompasses the ex vivo treatment of cells, e.g., cells derived from one of the transgenic animal described above, with a peptide aptamer library. The treated cells can then be studied in vitro or introduced into a host animal and monitored using art recognized techniques. For example, desired cell types or tissues that can be treated ex vivo and then reintroduced into a host animal following exposure to an aptamer library include, but are not limited to cells of the nervous system, muscle cells, and hematopoietic cells. In a preferred embodiment, hematopoietic cells lacking a gene needed for normal blood cell development or function, for example, a growth factor or a receptor, e.g., a T cell receptor, are contacted with an aptamer library and then introduced into a host animal, for example a host animal that has been treated so as to lack its normal blood cell repertoire (using, e.g., radiation). The animals treated with the cells exposed to the aptamer library are then monitored for the appearance of a desired phenotype (e.g., the repopulation of a particular blood compartment or outgrowth of a certain cell type), and such cells can then be isolated and used as a source of material for identifying an aptamer associated with the phenotype. Using the foregoing approach, the invention is suitable for screening peptide aptamers that are capable of affecting, e.g., cancers of the blood (e.g., mechanisms of leukemogenesis), immune cell function (e.g., T cell receptor function and/or other immune cell interactions), and various other diseases of the blood (e.g., hemochromatosis, or viral infections, e.g., an HIV infection).

3.5 Other Screening Advantages of the Invention

Another advantage of the present invention is integration of the primary screen with effective counter-screens that demonstrate the specificity of the phenotype. For example, cancer cells frequently undergo epigenetic changes that allow them to ignore normal growth regulatory signals, including apoptosis. In searching for aptamers that induce apoptosis in a given type of tumor cell, it is important to make sure that these aptamers do not induce apoptosis in normal cells.

In other examples, where an aptamer might be identified that modulates trafficking or transport of a particular protein, the invention provides the ability to incorporate counterscreens to determine that aptamer induced changes in localization are relatively specific to the phenotype of interest.

4.1 Use of Peptide Aptamers as a Prognostic Tool

The peptide aptamer of the invention can also be used as a prognostic tool for, for example, determining the likelihood of a cell to respond to a certain therapy based on the ability of an aptamer or panel of aptamers to alter the cell phenotype.

In a particular embodiment, the invention provides the ability to generate an apoptotic aptamer phenotype, i.e., a profile of aptamers that induces apoptosis and therefor represents important information about a given tumor cell. The aptamers allows for the categorization of any clonal population of tumor cells with respect to the most pertinent and important types of information, for example, how to destroy the cancer and with what selectivity and specificity.

In a clinical setting, for example, profiling of aptamer-associated apoptosis, conducted using, for example, a kit containing a panels of aptamers that can be used by investigators to draw correlations between the aptamer apoptosis phenotype and clinical prognosis, or serve as a predictive tool for the effectiveness of a given therapeutic strategy.

4.2 Use of Peptide Aptamers as a Diagnostic Tool

The methods and compositions of the invention can also be used for diagnostic purposes.

Accordingly, the retroviral aptamer libraries disclosed herein, or aptamers encoded by these libraries, can be packaged into kits with instructions for use. These kits can be used to screen for desirable aptamers using a format described herein for phenotyping, e.g., a cancer cell or tissue derived from, e.g., a biopsy sample. For example, a panel of vectors expressing peptide aptamers that induce apoptosis in cancer cells can be used for the “functional phenotyping” of tumor biopsy tissue, in which susceptibility to a particular aptamer can have predictive value for determining the efficacy of a certain treatment regimen. Alternatively, the kit may be used in conjunction with the cancer cell lines disclosed herein, other art recognized cell lines, or a combination thereof. In addition, the aptamer libraries may be used in conjunction with other screening technology involving, e.g., phage display and/or yeast two-hybrid systems for testing or validating a given aptamer. Still further, the methods and compositions described herein may also be used in conjunction with various art recognized gene chip technologies to, e.g., phenotype or diagnose a cancer. For example, the aptamer approach can be combined with gene chip technologies in order to enable the high-throughput quantitation of the expression of thousands of genes in a sample. This combined approach can be applied to the study of, e.g., diffuse large B-cell lymphoma (DLBCL), the most common subtype of non-Hodgkin's lymphoma, in order to discover identifiable differences in aptamer susceptibility and also gene expression patterns that correlate with and distinguish tumor proliferation rate, host response, and differentiation state of the tumor (Alizadeh et al., Nature 403:503-511 (2000)). Any of the forgoing composite diagnostic approaches are understood to be within the scope of the invention.

4.3 Use of Peptide Aptamers for Altering a Cell Phenotype

Aptamers can be identified that induce apoptosis in cells associated with other hyperproliferative disorders. These include, for example, prostatic hyperplasia in aging men and psoriasis. In addition, apoptosis is associated with diseases like osteoporosis, in which induction of osteoclast apoptosis (thus, decreasing cells that resorb bone), is of potential therapeutic benefit (Rezka et al., 1999). Conversely, aptamer screens can also be performed to identify inhibitors of osteoblast apoptosis. The therapeutic benefit of inhibition of apoptosis in the bone for generating needed cells is a desirable result (Plotkin et al., 1999).

4.4 Use of Peptide Aptamers in Gene Therapy

The therapeutic peptide aptamers capable of modulating a cell phenotype can be delivered to cells by methods of gene therapy. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Viral vectors include, for example, recombinant retroviruses, adenovirus, adeno-associated virus, and herpes simplex virus-1. Retrovirus vectors and adeno-associated virus vectors are generally understood to be the recombinant gene delivery system of choice for the transfer of exogenous genes in vivo, particularly into humans. Alternatively they can be used for introducing exogenous genes ex vivo into cells in culture.

A major prerequisite for the use of viruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271).

Thus, recombinant retrovirus can be constructed in which part of the retroviral coding sequence (gag, pol, env) is replaced by a gene of interest rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) Proc. Natl. Acad. Sci. USA 86:9079-9083; Julan et al. (1992) J. Gen. Virol. 73:3251-3255; and Goud et al. (1983) Virology 163:251-254); or coupling cell surface receptor ligands to the viral env proteins (Neda et al. (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). Thus, in a specific embodiment of the invention, viral particles containing a nucleic acid molecule containing a gene of interest, e.g., encoding a suitable peptide aptamer operably linked to appropriate regulatory elements, are modified for example according to the methods described above, such that they can specifically target desired cells.

Other methods relating to the use of viral vectors in gene therapy can be found in, e.g., Kay, M. A. (1997) Chest 111(6 Supp.): 138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N.Y. Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

For the particular treatment of a cancer, for example, following optional testing of the cancer biopsy sample, a vector encoding a predetermined aptamer can be injected directly into the tumor, or delivered in any other art-recognized manner of gene therapy. An advantage of the invention is that treatment of a given cancer in a subject with a vector encoding a therapeutic aptamer is an acute undertaking, which does not require perpetual expression of the introduced gene, which has been a difficulty in most other gene therapy approaches (Verma and Somia, 1997). In one approach, aptamer expressing viruses are used either as stand-alone therapeutics, or as adjuncts to other therapeutic regimens. For example, the viruses (e.g., retroviruses) can be injected directly into solid tumor sites to minimize the possibility of side-effects. In addition, aptamers can be identified that act in concert with other cancer therapeutic drugs or radiation therapy in a manner that lowers their effective doses, thereby decreasing toxicity or side effects of these treatments. Indeed, library screens can be undertaken in which aptamer expression results in apoptosis of a cell line in the presence of a sub-apoptosis inducing concentration of a conventional cancer therapeutic agent (e.g. tamoxifen or camptothecin).

4.5 Use of Peptide Aptamers as Therapeutics

Still further, the aptamer peptides of the invention, e.g., the conotoxin-derived peptides of the invention, i.e., conotides (and libraries thereof), are particularly well suited for use or for identifying an agent for any of the indications that the naturally-occurring conotoxins can be used for with the advantage being that the conotide of the invention has been improved in one or more aspects such as, e.g., affinity, e.g., to a cognate receptor, stability, or bioavailability.

Accordingly, conotides based on a backbone sequence from the A superfamily, i.e., conotoxins from the family, α, β, αA, and κA, can be used to modulate nAchRs, α₁-adenoreceptors, or voltage-gated K⁺ channels (see Table 1). Conotides based on a backbone sequence from the M superfamily, i.e., conotoxins from the family, μ and ψ, can be used to modulate Na⁺ channels or nAchRs . Similarly, conotides for the O superfamily, i.e., conotoxins from the families such as δ, ω, γ, μO, κ, Bromosleeper, and Conotoxin GS, can be used to modulate a variety of different ion channels (see Table 1). Other conotides based on a backbone sequence from a conotoxin from the S, T, P, and non-disulfide rich superfamilies (i.e., superfamilies that include σ, τ, χ, Conantokin, Contulakin, and Contryphan) can be used to, e.g., modulate the 5-HT₃ receptor, noradrenaline transporter, NMDA receptor or neurotensin receptor.

Disease or disorders which can be modulated (i.e., prevented or treated) using the conotides of the invention include, but are not limited to, pain, epilepsy stroke/ischemia, Parkinson's disease, dystonias, demyelinating disorders, congestive heart failure, cardiovascular disorders, neuromuscular disorders, chemotherapy-induced nausea and vomiting, psychiatric indications, cerebrovascular ischemia, cardiovascular disease, neurodegenerative disease, head injury, spasticity, neurotoxic injury associated with conditions of hypoxia, anoxia or ischemia which typically follows stroke, cerebrovascular accident, brain or spinal cord trauma, Alzheimer's disease, senile dementia, Amyotrophic Lateral Sclerosis, Multiple Sclerosis, Parkinson's disease, Huntington's disease, Down's Syndrome, Korsakoff's disease, schizophrenia, AIDS dementia, multi-infarct dementia, Binswanger dementia, psychiatric disorders, anxiety, major depression, manic-depressive illness, obsessive-compulsive disorder, schizophrenia and mood disorders, ophthalmic disorders, urinary incontinence, memory/cognition enhancement, i.e., treating memory, learning or cognitive deficits, analgesic agents, treatment of migraine, acute pain or persistent, non-addictive (e.g., non opioid based) pain management, convulsions, HIV infection and ophthalmic indications, agents and anti-pain agents for the treatment of acute and persistent pain, decrease bladder/sphincter dyssynergia (see, e.g., Jones et al., Exp Opin. Ther. Patents 11:603-623 (2001).

4.6 Use of Peptide Aptamers for the Research and Development of Other Therapeutics

Elucidation of aptamer targets can also serve as a powerful tool for the discovery of novel cellular targets that advance our understanding of the impacted cellular pathway/s. As such, the invention also encompasses retroviral aptamer libraries (e.g., in the form of kits), for use by basic researchers for genetic exploration of complex pathways in mammalian cells.

Accordingly, the invention can be used for the molecular classification of tumors and identification of previously undetected and clinically significant subtypes of cancer. In addition, the invention can be used as therapies that regulate or manage tumor growth (Balis, 1998).

Another embodiment of the invention includes the use of the aptamers as lead molecules for drug development. For example, using any art recognized molecular modeling techniques, an aptamer can be used for designing and synthesizing other molecules having the desirable function of the aptamer but also having other desirable traits such as cell solubility, potency, time-release properties, etc.

4.7 Use of Peptide Aptamers as Pharmaceutical Compositions

Another aspect of the invention pertains to pharmaceutical compositions of the peptide aptamers of the invention. In one embodiment, the composition includes an peptide aptamer in a therapeutically or prophylactically effective amount sufficient to alter, a desired activity, e.g., a cellular activity or a disorder or disease indication as described herein, and a pharmaceutically acceptable carrier. In another embodiment, the composition includes a peptide aptamer in a therapeutically or prophylactically effective amount sufficient to modify one or more of the modes of a action listed in Table 1 and a pharmaceutically acceptable carrier. A “therapeutically effective amount” refers to an amount effective, at dosages and for periods of time necessary, to achieve the desired therapeutic result, such as improved modulation of a receptor. A therapeutically effective amount of a peptide aptamer may vary according to factors such as the disease state, age, sex, and weight of the individual, and the ability of the aptamer compound to elicit a desired response in the individual. Dosage regimens may be adjusted to provide the optimum therapeutic response. A therapeutically effective amount is also one in which any toxic or detrimental effects of the aptamer compound are outweighed by the therapeutically beneficial effects. The potential toxicity of the aptamer compounds of the invention can be assayed using the cell-based assays or art recognized animal models and a therapeutically effective modulator can be selected which does not exhibit significant toxicity. In a preferred embodiment, a therapeutically effective amount of a peptide aptamer is sufficient to alter, and preferably inhibit, undesired cell growth (e.g., a cancer or a viral pathology or mechanism). A “prophylactically effective amount” refers to an amount effective, at dosages and for periods of time necessary, to achieve the desired prophylactic result, such as preventing or inhibiting undesired cell growth (e.g., a cancer or a viral pathology or mechanism). A prophylactically effective amount can be determined as described above for the therapeutically effective amount. Typically, since a prophylactic dose is used in subjects prior to or at an earlier stage of disease, the prophylactically effective amount will be less than the therapeutically effective amount.

It is to be noted that dosage values may vary with the severity of the condition to be alleviated. It is to be further understood that for any particular subject, specific dosage regimens can be adjusted over time according to the individual need and the professional judgment of the person administering or supervising the administration of the compositions, and that dosage ranges set forth herein are exemplary only and are not intended to limit the scope or practice of the claimed composition.

The amount of active aptamer compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

As used herein “pharmaceutically acceptable carrier” includes any and all solvents, dispersion media, coatings, antibacterial and antifungal agents, isotonic and absorption delaying agents, and the like that are physiologically compatible. In one embodiment, the carrier is suitable for parenteral administration. Preferably, the carrier is suitable for administration into the central nervous system (e.g., intraspinally or intracerebrally). Alternatively, the carrier can be suitable for intravenous, intraperitoneal or intramuscular administration. In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, monostearate salts and gelatin. Moreover, the aptamers can be administered in a time release formulation, for example in a composition which includes a slow release polymer. The active aptamer compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active aptamer compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

A peptide aptamer of the invention can be formulated with one or more additional compounds that enhance the solubility of the aptamer compound. Preferred compounds to be added to formulations to enhance the solubility of the aptamer compounds are cyclodextrin derivatives, preferably hydroxypropyl-γ-cyclodextrin. Drug delivery vehicles containing a cyclodextrin derivative for delivery of peptides to the central nervous system are described in Bodor, N., et al. (1992) Science 257:1698-1700.

Another preferred formulation for the aptamer compounds to enhance brain uptake comprises the detergent Tween-80, polyethylene glycol (PEG) and ethanol in a saline solution. A non-limiting example of such a preferred formulation is 0.16% Tween-80, 1.3% PEG-3000 and 2% ethanol in saline.

In another embodiment, a pharmaceutical composition comprising a peptide aptamer of the invention is formulated such that the modulator is transported across the blood-brain barrier (BBB). Various strategies known in the art for increasing transport across the BBB can be adapted to the aptamer compounds of the invention to thereby enhance transport of the aptamers across the BBB (for reviews of such strategies, see e.g., Pardridge, W. M. (1994) Trends in Biotechnol. 12:239-245; Van Bree, J. B. et al. (1993) Pharm. World Sci. 15:2-9; and Pardridge, W. M. et al. (1992) Pharmacol. Toxicol. 71:3-10). In one approach, the aptamer compound is chemically modified to form a prodrug with enhanced transmembrane transport. Suitable chemical modifications include covalent linking of a fatty acid to the modulator through an amide or ester linkage (see e.g., U.S. Pat. No. 4,933,324 and PCT Publication WO 89/07938, both by Shashoua; U.S. Pat. No. 5,284,876 by Hesse et al.; Toth, I. et al. (1994) J. Drug Target. 2:217-239; and Shashoua, V. E. et al. (1984) J. Med. Chem. 27:659-664) and glycating the aptamer compound (see e.g., U.S. Pat. No. 5,260,308 by Poduslo et al.). Also, N-acylamino acid derivatives may be used in an aptamer to form a “lipidic” prodrug (see e.g., U.S. Pat. No. 5,112,863 by Hashimoto et al.).

In another approach for enhancing transport across the BBB, the aptamer is conjugated to a second peptide or protein, thereby form ring a chimeric protein, wherein the second peptide or protein undergoes absorptive-mediated or receptor-mediated transcytosis through the BBB. Accordingly, by coupling the modulator to this second peptide or protein, the chimeric protein is transported across the BBB. The second peptide or protein can be a ligand for a brain capillary endothelial cell receptor ligand. For example, a preferred ligand is a monoclonal antibody that specifically binds to the transferring receptor on brain capillary endothelial cells (see e.g., U.S. Pat. Nos. 5,182,107 and 5,154,924 and PCT Publications WO 93/10819 and WO 95/02421, all by Friden et al.). Other suitable peptides or proteins that can mediate transport across the BBB include histones (see e.g., U.S. Pat. No. 4,902,505 by Pardridge and Schimmel) and ligands such as biotin, folate, niacin, pantothenic acid, riboflavin, thiamin, pryridoxal and ascorbic acid (see e.g., U.S. Pat. Nos. 5,416,016 and 5,108,921, both by Heinstein). Additionally, the glucose transporter GLUT-1 has been reported to transport glycopeptides (L-serinyl-β-D-glucoside analogues of [Met5]enkephalin) across the BBB (Polt, R. et al. (1994) Proc. Natl. Acad. Sci. USA 91:7114-1778).

A peptide aptamer can be coupled to such a glycopeptide to target the modulator to the GLUT-1 glucose transporter. For example, a modulator compound which is modified at its amino terminus with the modifying group Aic (3-(O-aminoethyl-iso)-cholyl, a derivative of cholic acid having a free amino group) can be coupled to a glycopeptide through the amino group of Aic by standard methods. Chimeric proteins can be formed by recombinant DNA methods (e.g., by formation of a chimeric gene encoding a fusion protein) or by chemical crosslinking of the aptamer to the second peptide or protein to form a chimeric protein. Numerous chemical crosslinking agents are known in the art. A crosslinking agent can be chosen which allows for high yield coupling of the modulator to the second peptide or protein and for subsequent cleavage of the linker to release bioactive aptamer. For example, a biotin-avidin-based linker system may be used.

In yet another approach for enhancing transport across the BBB, the aptamer is encapsulated in a carrier vector which mediates transport across the BBB. For example, the aptamer can be encapsulated in a liposome, such as a positively charged unilamellar liposome (see e.g., PCT Publications WO 88/07851 and WO 88/07852, both by Faden) or in polymeric microspheres (see e.g., U.S. Pat. No. 5,413,797 by Khan et al., U.S. Pat. No. 5,271,961 by Mathiowitz et al. and U.S. Pat. No. 5,019,400 by Gombotz et al.). Moreover, the carrier vector can be modified to target it for transport across the BBB. For example, the carrier vector (e.g., liposome) can be covalently modified with a molecule which is actively transported across the BBB or with a ligand for brain endothelial cell receptors, such as a monoclonal antibody that specifically binds to transferrin receptors (see e.g., PCT Publications WO 91/04014 by Collins et al. and WO 94/02178 by Greig et al.).

In still another approach to enhancing transport of the modulator across the BBB, the modulator is coadministered with another agent which functions to permeabilize the BBB. Examples of such BBB “permeabilizers” include bradykinin and bradykinin agonists (see e.g., U.S. Pat. No. 5,112,596 by Malfroy-Camine) and peptidic compounds disclosed in U.S. Pat. No. 5,268,164 by Kozarich et al.

A peptide aptamer of the invention can be formulated into a pharmaceutical composition wherein the aptamer is the only active compound or, alternatively, the pharmaceutical composition can contain additional active compounds. For example, two or more aptamer compounds may be used in combination.

In another embodiment, a pharmaceutical composition of the invention is provided as a packaged formulation. The packaged formulation may include a pharmaceutical composition of the invention in a container and printed instructions for administration of the composition for treating a subject having a disorder, e.g., undesired cell growth (e.g., a cancer) or a viral pathology.

Other features of the invention will be apparent from the following examples which should not be construed as limiting.

### EXEMPLIFICATION

Throughout the examples, the following materials and methods are used unless otherwise stated.

Materials and Methods

Cyclic Peptide for Drug Discovery

Unless otherwise noted, cyclic peptides for drug discovery using the methods and compositions of the invention can made according to Al-Obeidi et al., J. Med. Chem. 32:2555 (1989); Carpentier et al., J. Med. Chem. 32:1184 (1989); Rizo et al., Annu. Rev. Biochem. 61: 387 (1992); Hruby, Life Sci. 31:189 (1981); Hruby et al., Biochem J. 268: 249 (1990); Shan et al., J. Pharm. Sci. 86: 765-768 (1997); Takasaki et al., Nature Biotechnology, 15, 1266-1270 (1997); and Clarke et al., Biochemistry, 32:4322 (1993).

Cyclic Peptide Synthesis by Conventional Methods

Unless otherwise noted, cyclic peptides for use in the methods and compositions of the invention can made according to Kopple et al., J. Pharm. Sci. 61, 1345 (1972); Perlow et al., J. Org. Chem., 57, 4394 (1992)1; McGuiness et al., in “Peptides: Chemistry and Biology, Proceedings of the Fourteenth American Peptide Symposium” (P. T. P. Kuaumaya and R. S. Hodges, eds.), p.125. Mayflower Worldwide, Birmingham, UK, (1996); and Arttamangkul et al., Lett. Pept. Sci.m, 3:357 (1996).

Peptide Ligation Methods

Unless otherwise noted, peptide ligation methods for use in the methods and compositions of the invention can be performed according to Tam et a., Biopolymers (Peptide Science) 51:311-322 (1999); Dawson et al., Science, 266:776 (1994); and Tam et al., Proc. Natl. Acad. Sci. U.S.A., 92:12485 (1995).

Cyclic Peptide Synthesis by Thioester Ligation

Unless otherwise noted, cyclic peptide synthesis by thioester ligation for use in the methods and compositions of the invention can made according to Zhang et al., J. Am. Chem. Soc., 119:2363-2370 (1997); Tam et al., Tetrahedron Lett. 38:5599 (1997); Camarero et al., Chem. Inlt Ed., 37:347-349 (1998); and Tam et al., Protein Sci. 7:1583-1592(1998); Zhang et al., J. Am. Chem. Soc., 121: 3311-3320 (1999); Sun et al., Org. Lett., 3 (11):1681-1684 (2001); Tam et al., J. Am. Chem. Soc., 121:4316-4324 (1999); and Yan et al., J. Am. Chem. Soc., 123:526-533 (2001).

Cyclic Peptide Library Production in Retroviruses

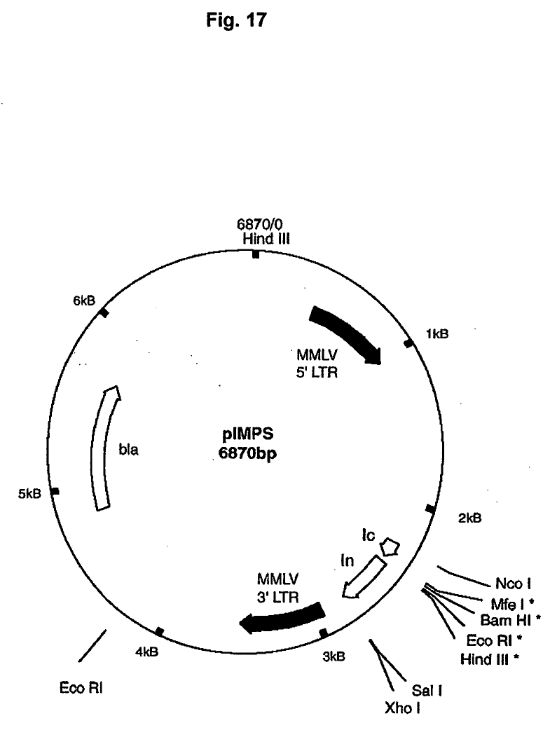

Oligonucleotides were synthesized using standard nucleic acid synthesis to typically encode 9 amino acid residues structured as a cyclic-Ser-X-X-X-Gly-Pro-X-X-X (SEQ ID NO: 1). The Serine residue is an acceptor of protein splicing, each X is represents any amino acid residue (except for a stop codon) and the Glycine and Proline (Gly-Pro) motif is designed to facilitate the cyclization of the peptide. The synthetic oligonucleotides were cloned in between the Mfe I and the Hind III sites between the N-terminal (In) and C-terminal (Ic) intein fragments of the DnaE gene engineered into a murine retroviral vector (i.e., the pIMPS vector, see also FIG. 17, SEQ ID NO: 2). Recombinant products were electroporated into Top10 bacteria to generate a library having a complexity of approximately 10⁸ independent colonies (clones).

Cell transfection and Virus Production

To generate infectious virions, 293T cells were transfected with 15 μg of retroviral peptide library DNA and 5 μg of helper virus DNA (EcoPac vector) using standard calcium phosphate precipitation. Briefly, 62.5 μl of calcium chloride (2M) was added to the DNA, diluted in 425 μl of distilled water, and a precipitate was obtained by adding 500 μL of Hepes buffer 2× dropwise while vortexing slowly. The precipitate was then added to 293T cells in 5 ml of DMEM+10% FCS and allowed to incubate at 37° C. for 7 hours. The cells were then washed and incubated with fresh media and supernatants containing infectious virions were collected at 24 and 48 hours post-transfection. Typically, packaging of the retroviruses was accomplished by transfecting a proviral vector (e.g., pIMPS) into a commercially available packaging cell line such as, e.g., EcoPack-293 (HEK293 cells; Cat. No. C3200-1; available from Clontech). For producing pantropic virus, co-transfections were performed using the VSV-G plasmid and GP-293 packaging cell line (available from Clontech; Cat. No. K1063-1)

Transduction of Retroviral Libraries

Cells were plated at 5×10⁵ cells per 10 cm diameter plate 24 hours prior to retroviral transduction infected using supernatant from 293T cells transfected with the library. Infections were carried out for 6 hours in the presence of 8 mg/ml of polybrene and continued overnight after the addition of additional growth media. The following day, cells were rinsed once with PBS and allowed to recover one day in growth media before phenotypic selection.

Titering of Retroviral Libraries

The titer of retroviral libraries was carried out either by packaging retroviral vectors encoding green fluorescent protein (GFP), transduction of these retroviruses, and quantitation of the number of cells demonstrating fluorescence by microscopy. Preferably, retroviral titer is also more directly established by Southern blot analysis of transduced cells. Serial dilutions of packaged retroviral stock are used to transduce host cells, and copy number is assessed by quantitative Southern blot; titer is extrapolated by determining the viral dilution at which transduction of host cells occurs at an average of one copy per cell.

Titering of EMCV Virus

Virions were produced by infection of L929 cells in a large 150 cm² flask with 0.5 ml aliquot of viral supernatant which were then allowed to incubated overnight. The next day, supernatants bearing infectious virions were harvesting and titered using a plaque forming unit (PFU) assay as described below. Briefly, murine L929 cells were plated as an ˜80% confluent monolayer and inoculated with a 100 μl aliquot of various virus dilutions, incubated and then covered in media and agar and allowed incubate for 2 days. Cells were then fixed with formalin, the agar was removed, and cells attached to the plate were stained with 2% crystal violet/20% ethanol to allow for enumeration of the clear zones or plaque forming units (PFU) formed by as a result of successful infection by virus.

Titering of EMCV Virus by Cell Viability Assay: An Alternative High-Throughout Method

Cells (L929) were plated at a density of 10⁴ cells/well (in 96 well plates), incubated overnight, and then infected with cell supernatants or serial dilutions of EMCV preparations in 100 μl of RPMI+10% FBS. Infections were performed in a 37° C. humidified CO2 incubator for about 20 hours. The viability of cells after the lytic infection of EMCV was measured using a colorimetric assay, according to the manufacturer's instructions (CellTiter 96® AQᵤₑₒᵤₛ One Solution Cell Proliferation Assay; Promega).

Immunoblot Analysis

Cellular protein extracts were prepared by harvesting the cells, after transfection or transduction, washing the cells once with PBS and resuspending the cells in 1 ml of cold lysis buffer (50 mM Tris. HCl (pH 7.5), 150 mM NaCl, 1 mM EGTA, and 0.5% Triton X-100). The cells were then incubated on ice for 15 min, vortexed, centrifuged at low speed to eliminate nuclei, and the resultant supernatant was recovered for analysis. Protein samples were analyzed by SDS-PAGE gel (4-20% polyacrylamide pre-cast gel, Invitrogen) followed by immunoblotting onto nitrocellulose membranes using standard techniques. Proteins of interest were then resolved using immunodetection with an appropriate primary antibody (e.g., murine anti-HIS or murine anti-GFP), blotting with a secondary antibody coupled to horseradish peroxidase (anti-mouse-HRP diluted at 1/2000), and visualized using ECL (Enhanced ChemoLuminescence; Amersham).

Throughout the examples, unless otherwise indicated, the practice of the present invention will employ conventional techniques of chemistry, molecular biology, microbiology, recombinant DNA technology, cell culture, and animal husbandry, which are within the skill of the art and are explained fully in the literature. See, e.g., Sambrook, Fritsch and Maniatis, Molecular Cloning: Cold Spring Harbor Laboratory Press (1989); DNA Cloning, Vols. 1 and 2, (D. N. Glover, Ed. 1985); Oligonucleotide Synthesis (M. J. Gait, Ed. 1984); PCR Handbook Current Protocols in Nucleic Acid Chemistry, Beaucage, Ed. John Wiley & Sons (1999) (Editor); Oxford Handbook of Nucleic Acid Structure, Neidle, Ed., Oxford Univ Press (1999); PCR Protocols: A Guide to Methods and Applications, Innis et al., Academic Press (1990); PCR Essential Techniques: Essential Techniques, Burke, Ed., John Wiley & Son Ltd (1996); Nucleic Acid Hybridization (B. D. Hames and S. J. Higgins, Eds. 1984); the series Methods In Enzymology (Academic Press, Inc.), particularly Vol. 154 and Vol. 155 (Wu and Grossman, Eds.; Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); Coffin, et al., Retroviruses, Cold Spring Harbor Laboratory Press (1997); Weiner, et al., Chemical and Structural Approaches to Rational Drug Design (Pharmacology and Toxicology), CRC Press (1995); Bodanszky, et al., Peptide Chemistry: A Practical Textbook, Springer Verlag Press (1993); McClelland, et al., Expression Genetics: Accelerated and High-Throughout Methods (Biotechniques Update Series), Eaton Pub Co., (1999); Devlin, High Throughout Screening, Marcel Dekker Press, (1997); Sucholeike, High Throughput Synthesis, Marcel Dekker Press (2001); Murray, Principles and Practice of High Throughput Screening, Blackwell Science, Inc. (2001); Weiner, et al., Biological Approaches to Rational Drug Design (Pharmacology and Toxicology), CRC Press (1994); Roth, Methods in Cell Biology: Protein Expression in Animal Cells, Academic Press, (1994), Large-Scale Mammalian Cell Culture Technology, Lubiniecki, A., Ed., Marcel Dekker, Pub., (1990); Molecular and Cell Biology of Yeasts, Yarranton et al., Ed., Van Nostrand Reinhold, Pub., (1989); Yeast Physiology and Biotechnology, Walker, G., John Wiley & Sons, Pub., (1998); Baculovirus Expression Protocols, Richardson, C., Ed., Humana Press, Pub., (1998); Methods in Plant Molecular Biology: A Laboratory Course Manual, Maliga, P., Ed., C.S.H.L. Press, Pub., (1995); Current Protocols in Molecular Biology, eds. Ausubel et al., John Wiley & Sons (1992) and Manipulating the Mouse Embryo, Hogan et al., C.S.H.L. Press, Pub (1994)).

### Example 1

### Methods and Compositions for Producing Cyclic Peptide Aptamer Libraries using Protein Splicing

In this example, methods and compositions are described for the production and phenotypic screening of peptide aptamers that undergo spontaneous cyclization through intein-mediated protein splicing.

An intein derived from Synechocysti (see also WO/36093) is used as proof of principle that intein sequences can be used to bring about targeted protein splicing in mammalian cells for the purpose of creating a cyclic polypeptide, e.g., a cyclic peptide aptamer (e.g., cPEP).

In brief, the pMFG vector, which is based on the Moloney Murine Leukemia Virus (MMLV), was modified to generate the pIMPS vector (SEQ ID NO: 2). Using methods well known in the art, the pIMPS vector, and derivatives thereof, can be transfected into appropriate cell lines for packaging and production of recombinant retrovirus. The pIMPS vector (see FIG. 17) was designed to encode and express an I_{C} intein splicing element, followed by a multiple cloning site and an -I_{N} intein splicing element. (The endogenous Hind III m site in pMFG was removed by Hind III cleavage, Klenow fill-in, and religation.) A split intein, derived from the two DnaE genes of Synechocysti PCC6803 (Accession Nos: D90917 AB001339 and D90904 AB001339), was incorporated into the resulting vector by standard cloning techniques. A schematic drawing of the pIMPS vector is shown in FIG. 17. (see also SEQ ID NO: 2).

A general strategy for the insertion of nucleotide inserts encoding library peptides or proteins that undergo intein-mediated splicing upon intracellular expression using the pIMPS vector is described below and in Table 3. The pIMPS vector is digested with Mfe I and Hind III. The I_{C}-I_{N} junction at the Hind III site was engineered to create a frameshift in the absence of an insert between I_{C} and I_{N}. In addition, the pIMPS vector is designed not to encode key catalytic residues at the I_{c}/Iₙ insertion site. Instead, the 5′ end of the aptamer peptide or polypeptide insert is designed to be compatible with the Mfe I overhang and digesting with Mfe I, EcoR I, or Apo I enzymes will generate a suitable 5′ ‘AATT’ extension. The ‘AAT’ of the extension is the codon for the catalytically-required asparagine of I_{C}. The remaining ‘T’ of the overhang is the first nucleotide of the I_{C} nucleophile. Depending on the restriction enzyme used to digest the insert, and therefore the nucleotide immediately following the restriction site, the resultant I_{C} nucleophile will vary as provided below in Table 3.

TABLE 3

| | Codon/I_{C} | splicing |
|---|---|---|
| Insert restriction site (5′) | nucleophile | competent |
| CAATTGy (Mfe I) | TGY/C | yes |
| CAATTGa (Mfe I) | TGA/STOP | no |
| CAATTGg (Mfe I) | TGG/W | yes |
| GAATTCn (EcoR I) | TCN/S | yes |
| RAATTYn (Apo I) | TTN/F or L | yes |
| RAATTYn (Apo I) | TCN/S | yes |

The 3′ end of the insertion site of pIMPS is designed to contain a Hind III site, but lacks coding sequences for cysteine and leucine residues that are required for optimal splicing. Accordingly, the optimal aptamer insert ends with the following sequence: TGY YTA AGC TT (SEQ ID NO: 5), has a Hind III site and sticky end, and its insertion reconstitutes the correct number of nucleotides and codons necessary for I_{N} to function in the intein splicing reaction. If desired, the cysteine can be converted to other residues to modulate activity by changing the ‘TGY’ sequence to a codon encoding another amino acid. Combining the guidelines from above, the “top” or “coding” strand of a “functional” pIMPS insert (following digestion or other modifications necessary to prepare it for insertion into the pIMPS vector) has the following sequence: AAT TSY (NNN)_{X} TGY YTA (SEQ ID NO: 6), and therefore has a total length of 3X+12 where X is the number of random amino acid residues desired in the translated and circularized final peptide product.

Using the methods described above, a library of self-cyclizing random peptides was constructed in the following manner. Sequences encoding and flanking the peptide aptamer sequence were designed to be cleavable by EcoR I and Hind III sites, which is then digested and inserted in frame with the upstream I_{C} coding sequences and the downstream I_{N} coding sequences:

| | |
|---|---|
| G AAT TCT (NNN)ₓ TGC TTA AGC TT | (SEQ ID NO: 7) |
| N S X C L S | (SEQ ID NO: 8) |

Accordingly, the complete nucleotide sequence will have a total length of 3X+18 basepairs (counting from the ‘G’ in EcoR I to the second ‘T’ in Hind III) plus any additional residues used to flank the EcoR I and Hind III sites. The resulting plasmids are then transformed and propagated in E. coli using standard techniques. The plasmids are then harvested, transfected into packaging cells and the resultant virions produced are used to transduce the random peptide library into cells in which expression and subsequent splicing of the library inserts is allowed to occurs in cells. The cells containing peptides that bring about a phenotypic change are then identified as described herein.

Upon completion of requisite screening and counter-screening steps or procedures, DNA from the population of cells possessing the desired phenotype is harvested by standard methods. For example, the PUREGENE DNA Isolation kit from Gentra Systems yields genomic DNA in sufficient amounts and purity to generate PCR products from as few as 100 cells. The primer pair for retrieving the sequence encoding the identified candidate aptamer having functional activity is:

| | |
|---|---|
| (forward, for I_{C}, termed ENOL 351: | |
| GCCGTCTAGAGCCGCCATGGTTAAAGTTATCGGT | (SEQ ID NO: 9) |
| CGTCGTTCCCTC (Nco I site in bold) | |
| (reverse, for I_{N}, termed ENOL 354) | |
| GCTAGTCGACTTATTTAATTGTCCCAGCGTCAAG | (SEQ ID NO: 10) |
| TAATGGAAAGGG (Sal I site in bold). | |

The resultant PCR product using this primer pair encodes the entire ORF (open reading frame) of a retrovirally transduced I_{C}-CP-I_{N}, flanked by Nco I and Sal I cloning sites. In the example shown, the expected PCR product is 533 bp with a 507 bp ORF (aptamer library insert). This insert can be re-cloned into pIMPS (or any other vector appropriate for expression in mammalian cells or repackaging into a secondary library. Alternatively, or in parallel, the amplified insert can be sequenced using standard techniques.

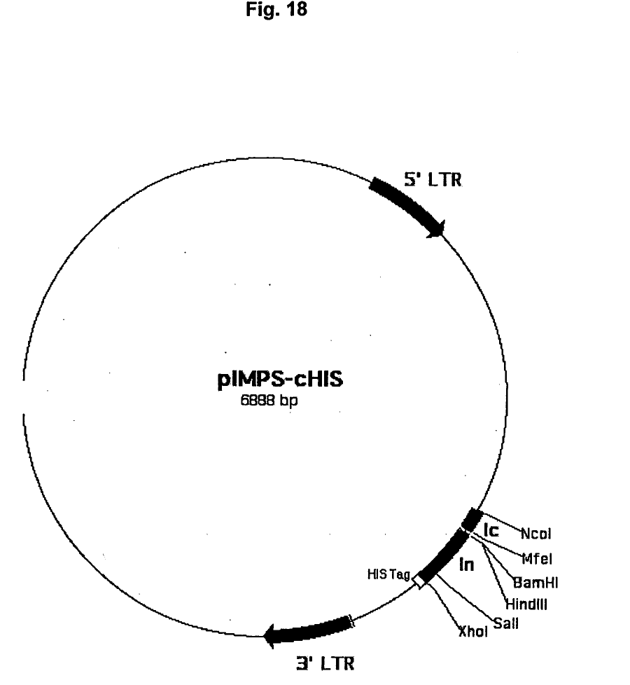

In addition to the pIMPS vector, a modified derivative was produced to allow facile monitoring of intein splicing reaction. This vector, referred to as the pIMPS-cHIS and shown in FIG. 18, contains I_{C} encoding sequences and a site for insertion of peptide aptamer coding sequences identical to those of pIMPS. In addition, a sequence encoding a 6×HIS tag (i.e. six consecutive histidine codons) was added to the I_{N} moiety by insertion of a short synthetic fragment.

This modification of pIMPS-cHIS provides two useful features. First, cell lysates from cells transfected with a pIMPS-cHIS plasmid into which peptide or protein coding sequences have been cloned into the insertion site, can be analyzed by standard methods (i.e., immunoblot) to confirm expression of an I_{C}-Insert-I_{N} protein. The presence of a 6×HIS tagged protein confirms that an insert (or family of inserts in the case of a peptide library) is present in pIMPS-cHIS, since, as described above, an appropriate insertion event is necessary to adjust the reading frame downstream of the insertion site to encode an In moiety that contains a polyhistidine tag, in the case of pIMPS-cHIS.

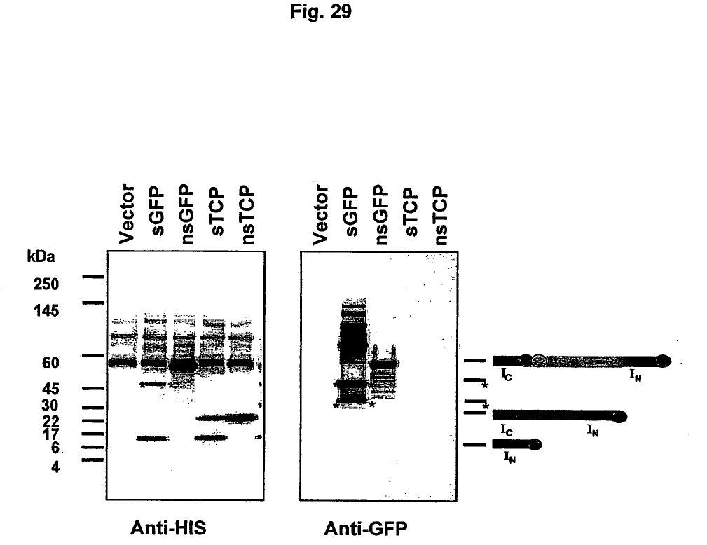

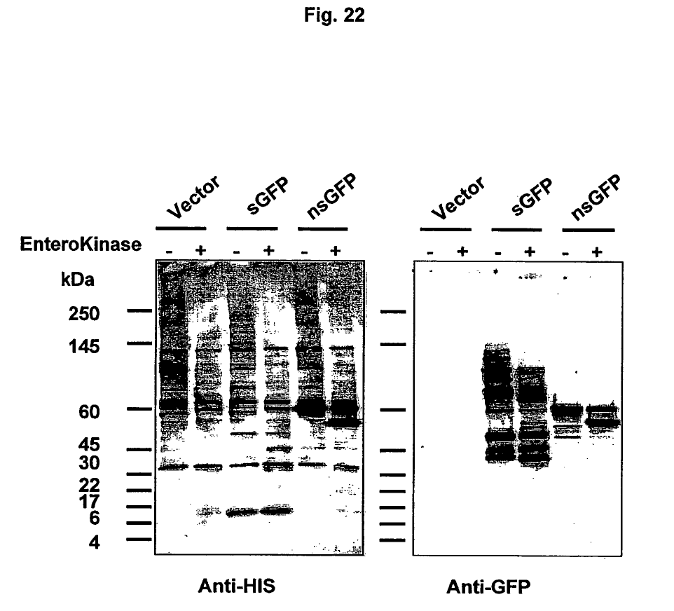

Results of intein mediated splicing of proteins or peptides expressed by the pIMPS-cHIS vector in mammalian cells are shown in FIG. 19. pIMPS-cHIS vectors into which coding sequences for the green fluorescent protein (GFP) or a short test cyclic peptide (TCP) were cloned and expressed in frame between the I_{C} and I_{N} intein elements. Cell lysate samples in which splicing-competent forms of these respective precursors were transfected are denoted as sGFP and sTCP, and non-splicing controls are denoted as nsGFP and nsTCP. Unprocessed and processed forms of these proteins are detected in the left panel by probing by immunoblot with an antibody that recognizes the 6×HIS tag at the carboxy-terminus of the I_{C}-INSERT-I_{N}-HIS precursor and the smaller In-HIS splicing product, respectively (FIG. 22).

Analogous experiments can be performed analyzing lysates of cells that have been transduced by recombinant pIMPS-cHIS-derived retroviruses or retroviral libraries.

### Example 2

### Incorporation of β-Turn Motifs to Improve Splicing Efficiency and Intracellular Peptide Cyclization through Intein-Mediated Protein Splicing

In this example, methods and compositions for producing intein peptide aptamers that comprise β-turn motif for improved intein-mediated protein splicing, are described.

Libraries encoding random peptides in which all residues in the cyclized product are random, with the exception of residues that are required for splicing itself (i.e., the serine or cysteine nucleophiles that are necessary for intein-mediated splicing, and that remain in the cyclic peptide product after the reaction), can also be engineered to contain one or more β-turn motifs. For example, Gly-Pro or Pro-Gly residues can be fixed within the random peptide.

| | |
|---|---|
| AAT NNN NNN NNN NNN GGN CCN NNN NNN NNN NNN NNN | SEQ ID NO: 12 |
| NN NNN NNN NNN CCN GGN NNN NNN NNN NNN NNN TCGA | SEQ ID NO: 13 |
| X X X X G P X X X X | SEQ ID NO: 14 |

Peptide libraries containing this sequence have the following advantages which are: (i) the cyclic peptide product contains a structural motif that serves as context for the remaining random residues, and (ii) the flexibility within the cyclic peptide coding sequence improves the protein splicing efficiency of the modified peptide flanked by intein domains (I_{C}-cPEP-I_{N}) (Cochran et al., Proc. Natl. Acad. Sci. USA 98: 5578-5583 (2001)). This was demonstrated with the following experiment.

The following Hind III compatible/Mfe I compatible double-stranded oligonucleotide sequences were cloned into the Mfe I-Hind III sites of the pIMPS-cHIS vector:

| | |
|---|---|
| AAT TTG TTC CTG TAC GGG CCC TGG AAA GAC TTG TTA | SEQ ID NO: 15 |
| AC AAG GAC ATG CCC GGG ACC TTT CTG AAC AAT TCGA | SEQ ID NO: 16 |
| L F L Y G P W K D L | SEQ ID NO: 17 |
| AAT TTG TTC CTG TAC GCC GCC TGG AAA GAC TTG TTA | SEQ ID NO: 18 |
| AC AAG GAC ATG CGG CGG ACC TTT CTG AAC AAT TCG AAA | SEQ ID NO: 19 |
| L F L Y A A W K D L L | SEQ ID NO: 20 |
| (G) (P) | |
| AAT TCG TTC CTG TAC GCC GCC TGG AAA GAC TGC TTA | SEQ ID NO: 21 |
| GC AAG GAC ATG CGG CGG ACC TTT CTG ACG AAT TCG A | SEQ ID NO: 22 |
| S F L Y A A W K D C L | SEQ ID NO: 23 |
| (G) (P) | |

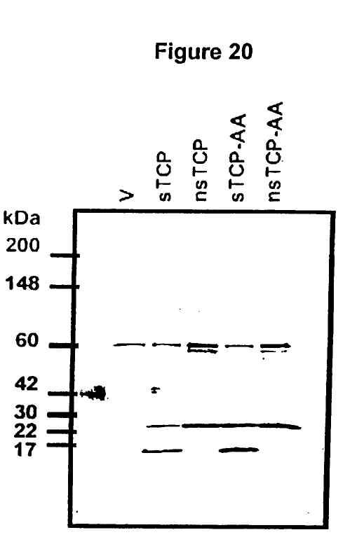

The resulting plasmids were transfected into 293T cells, and splicing efficiency was determined by immunoblot detection of the I_{N}-HIS reaction product in cell lysates of the transfected cells (see FIG. 20). Comparison of the ratio of unspliced products in extracts from cells expressing a test cyclic peptide (TCP) containing a Gly-Pro β-turn structure compared to an analogous peptide in which the Gly-Pro β-turn structure is substituted by Ala-Ala, were measured and indicated a splicing efficiency of the peptide containing a Gly-Pro β-turn structure to be approximately 30% more efficient.

### Example 3

### Methods and Compositions for Producing Cyclic Peptide Aptamer Libraries Fused to Green Fluorescent Protein (GFP)

In this example, methods and compositions for producing peptide aptamers fused to an intein that can undergo protein splicing and are fused to green fluorescent protein (GFP), are described.

While several contexts for insertion of random amino acid domains into GFP have been described, these strategies have focused on identification of looped or structurally constrained domains within GFP that are presented and accessible on the outer surface of the GFP protein, which can tolerate insertion of exogenous amino acids without decreasing fluorescence (see U.S. Pat. Nos. 6,025,485 and 6,180,343). Several such domains within GFP have been identified and used in this manner. However, in each case, the fluorescent properties of GFP are significantly lower than the native protein: even in the best cases, fluorescence is decreased by at least an order of magnitude upon insertion of peptide coding regions within looped domains of GFP.

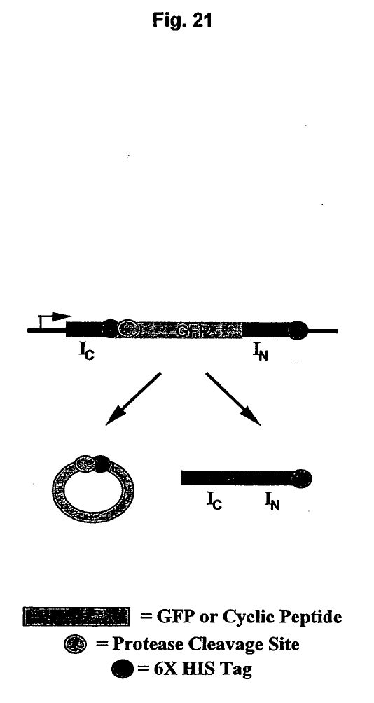

The present invention provides specific regions of the green fluorescent protein which can be altered to accommodate an aptamer sequence without any deterioration of the fluorescent activity of the GFP. The GFP protein is composed of a series of β-strands arranged as a cylinder, with the free N- and C-termini of the protein projecting upward, away from the “top” of the barrel structure (see FIG. 21). The projection of these free N- and C-termini facilitate the fusion of heterologous proteins to either end of GFP without adversely affecting the function of the fused protein or the fluorescent properties of GFP. This invention uses I_{C} and I_{N} intein splicing domains that are fused to the amino and carboxy termini of native GFP. These intein domains then carry out peptide ligation, resulting in a covalently closed, cyclized GFP protein with no free N- or C-terminus, and producing liberated I_{N} and I_{C} products.

This reaction is used to generate libraries in which random peptides are presented in a constrained loop that extends across the space that normally exists between the N- and C-termini of native GFP. This is carried out as follows: nucleotides encoding random amino acids are inserted into a retroviral vector downstream of an I_{C} coding sequence, and upstream of the beginning of GFP. Sequences encoding a second set of random amino acids are positioned in frame following the GFP coding sequence, followed by coding sequences for the I_{N} intein domain. These respective random amino acid sequences, which flank the GFP coding region, and which are themselves flanked by I_{C} and I_{N}, are referred to respectively as “X” and “Y”. Accordingly, these random elements are expressed in the cell as part of a contiguous precursor protein (I_{C}-X-GFP-Y-I_{N}). The I_{C} and I_{N} intein elements of this precursor protein undergo splicing to produce a cyclic GFP product that joins the X and Y peptide domains, generating a contiguous (XY) peptide that extends across the top of the GFP barrel. The resulting GFP product has no free N- or C-termini, but instead presents the peptide aptamer as a constrained peptide fused to GFP.

By expressing a chimeric GFP, intein-mediated splicing results in a covalent cyclization of a GFP protein in which the aptamer is presented in a way that does not intervene in any GFP sequence such that its fluorescence is diminished. The expression of cyclized GFP proteins that present random peptide aptamers can then be accurately monitored by the presence of green fluorescent protein activity using standard techniques, e.g., fluorescent activated cell sorting (FACS).

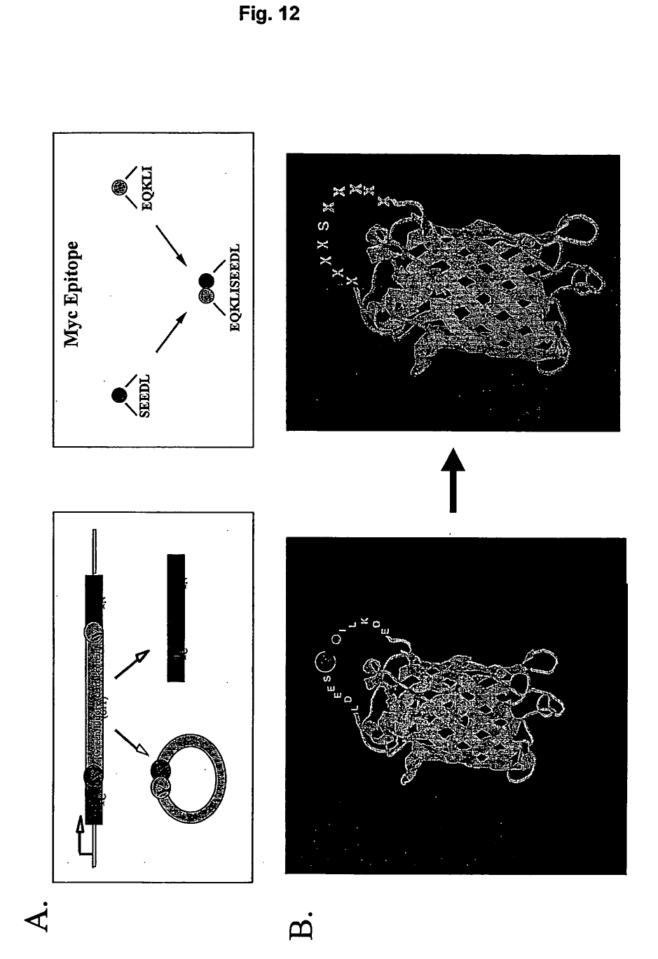

As a proof of principle, the myc epitope consisting of the amino acid sequence EQKLISEEDL (SEQ ID NO: 11) that is recognized by the 9E10 monoclonal antibody was genetically fused to GFP using standard genetic engineering. Specifically, oligonucleotide linkers were designed to place half of the myc epitope in front of the GFP reporter protein and the other half behind the GFP protein, i.e., as SEEDL [green fluorescent protein (GFP)] EQKLI and as shown in FIG. 12 (SEQ ID NOS: 24-25). The subsequent intein-mediated splicing reaction generates a contiguous EQKLISEEDL polypeptide sequence, which is presented as a novel loop “across the top” of the GFP, resulting in a restoration of the myc epitope sequence which is not present in the absence of cyclization. This can be confirmed using the 9E10 monoclonal antibody that only detects the restored, i.e., correctly spliced sequence of the myc epitope.

To determine that the above myc GFP fusion encoded the expected protein, cells were transfected with a vector also containing a histidine epitope (for purification and immunodetection; SEQ ID NOS: 3-4) and transduced using packaged retrovirus and the resultant HIS tagged proteins were harvested and purified using standard NiNTA chromatography followed by elution with imidazole. The purified HIS tagged proteins were then cleaved with enterokinase protease and analyzed by SDS-PAGE and determined by immunoblot (FIG. 22).

### Example 4

### Methods for Producing Conotides

In this example, methods and compositions for making cyclized conotoxins or conotides, by intein-mediated protein splicing and/or disulfide bond replacement, are described.

Briefly, conotoxins or conotides are generated either through chemical cyclization of a linear synthesized peptide, or through the intein-mediated splicing as described herein. In either approach, the naturally occurring proximity of the amino- and carboxy-termini of the conotoxin peptide are maintained through a covalent (amide) bond, thereby rendering these molecules structurally constrained but protease resistant. In the case of cyclic peptides (conotides) derived from naturally occurring conotoxins, the cyclic peptides produced in this manner are tested for improved activity corresponding to that of the naturally occurring conotoxin peptide. For example, the cyclized form of the α-conotoxin GI peptide sequence (ECCNPACGRHYSC; SEQ ID NO: 26) and the improved activity of this cyclized form compared to that of the naturally occurring α-conotoxin GI peptide can be measured by standard binding assays.

In one embodiment, forms of these cyclic peptides are produced in which one or more cysteine pairs are eliminated. In these peptides, the stable structural constraints that are normally provided by cysteine-mediated disulfide bonds are instead provided by the amide closure. For example, in the cyclized α-conotoxin GI peptide, the cysteine residues at positions 3 and 13, which normally form a structurally important disulfide linkage, are replaced in the cyclic peptide with other non disulfide forming amino acids (i.e. alanine, methionine, serine, threonine, glycine, leucine, isoleucine, valine), and the resulting cyclized peptides are tested for α-conotoxin GI activity. The same process is carried out for replacement of the cysteines that mediate the disulfide bridge between residues 2 and 7 of the α-conotoxin GI peptide. In addition to these strategies that examine replacement of these disulfide bond functions separately, peptides can also be generated and tested that replace all four structurally important cysteines, thereby eliminating both endogenous disulfide bonds, for improved activity.

Within the process described above, in which replacement of one or more disulfide bonds is examined systematically, additional replacement strategies are contemplated. In the case of the α-conotoxin GI peptide, replacement of disulfide bond forming cysteine pairs with aromatic amino acid residues (e.g. tryptophan, tyrosine, or phenylalanine) allows ring stacking, thereby stabilizing critical secondary structures within the cyclic peptide, but alleviating the need for one or both disulfide bridges. Such ring stacking by aromatic residues has been shown to facilitate the formation of stable monomeric β-hairpins in linear peptides (Cochran et al., 2001) which sponsors the formation of functional conotoxin structures in conotoxin-derived peptides where one or more cysteine pairs have been replaced. This added structural organization associated with cyclization of the conotides augments the efficacy of aromatic ring stacking in forming secondary structures associated with conotoxin activity.

An alternative to this approach is the use of salt bridges to fulfill the role of cysteine mediated disulfide bonds. For example, in the case of the α-conotoxin GI peptide, a positively charged amino acids such as lysine or arginine is used to replace the cysteine in position 3, in composed of two α units and one each β, γ, and δ unit. Other α and αA-conotoxins, which are composed of several α and β units, target neuronal nAChRs

Conotides based on the foregoing preferably have an insertion of charged amino acids that result in changes in the affinity of the conotide for a particular target, as for example, compared to a conotoxin control. For instance, in α-conotoxin SI (ICCNPACGPLYSC-NH2; SEQ ID NO: 33), Pro9 replaces the Arg9 found in α-conotoxin GI. This change results in α-conotoxin GI that are lethal to mammals at low concentrations. α-conotoxin SI, however, while active in T. californica, produces essentially no effect in mammals. The sequence differences between the conotoxins also results in different affinity for different sites on the same receptor. α-conotoxin SI does not distinguish between the α/δ and α/γ sites in T. californica electric organ, but α-conotoxin GI finds the α/δ site preferable to the α/γ site. The exemplary α-conotoxin GI and its derivatives described above can be screened for receptor binding and specificity.

### Example 6

### Methods and Compositions for Producing and Screening Conotide Libraries

As described above, the conotoxin peptides, or more particularly, structural motifs found in naturally occurring conotoxin peptides, are privileged structures that have been evolutionarily selected to be ideally suited for binding to receptors, including nAch receptors, as well as various ion and voltage gated channels. This invention contemplates the use of recombinant DNA techniques to generate and phenotypically screen random peptide libraries in which residues of a given conotoxin that do not have important structure-determining roles are replaced by random amino acids to produce a conotide library. Conotide libraries are generated by insertion of the coding sequences below into a vector suitable for expression of the encoded peptide. Although this expression can be carried out in a variety of cells (e.g. bacteria, yeast, insect cells or mammalian cells), in preferred embodiments, retroviral vector systems are used to generate and express the random peptide libraries in mammalian cells, where phenotypic screening can be carried out.

Conotoxin peptides, as they exist in nature, are secreted and act extracellularly upon target membrane proteins of cells within the prey organism. Thus, although the targets of individual conotoxins may vary, the tasks for which they were evolutionarily selected did not require that they act upon intracellular targets. However, in consideration of the observation that conotoxin-like molecules are uniquely suited for highly specific interaction with various membrane receptors and channels, intracellular expression of conotoxin-like peptides, accompanied by screening of cell populations expressing random peptides that incorporate important to maintain a cysteine or serine nucleophile at the first position of the conotide coding sequence, in frame and immediately downstream of the I_{C} intein splicing element, in order for intein splicing to occur.) Exemplary amino acid and oligonucleotide sequences for the α-conotoxin GI peptide are listed below:

| | |
|---|---|
| tgc tgc aac ccg gcg tgc ggt cgt cat tat agc tgc ggt agc gaa | (SEQ ID NOS: 27, 28) |
| C C N P A C G R H Y S C G S E | |
| tgc ggt cgt cat tat agc tgc ggt agc gaa tgc tgc aac ccg gcg | (SEQ ID NOS: 29, 30) |
| C G R H Y S C G S E C C N P A | |
| tcg tgc ggt agc gaa tgc tgc aac ccg gcg tgc ggt cgt cat tat | (SEQ ID NOS: 31, 32) |
| S C G S E C C N P A C G R H Y | |

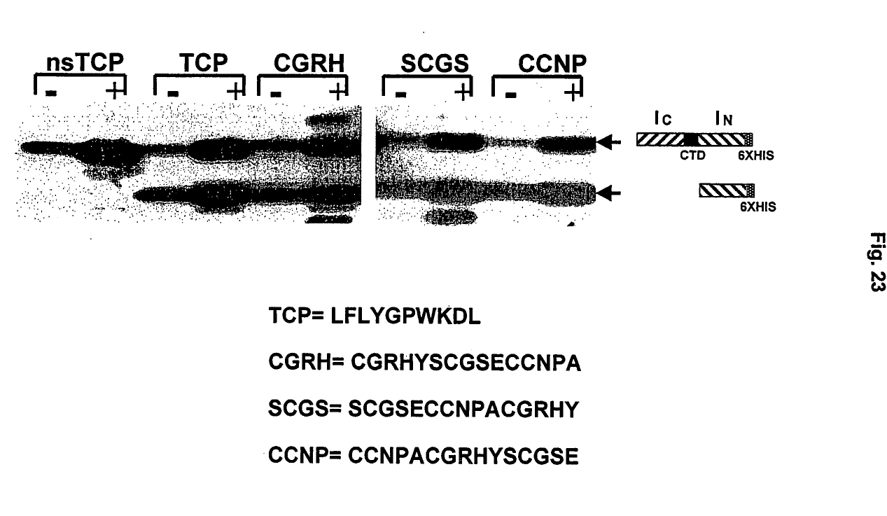

An analogous approach, using an appropriate bacterial expression vector, such as the pET derived vector, can be used to generate such conotoxin-derived peptides in bacteria. Results from the expression and processing of these respective intein flanked conotides (i.e., I_{C}-conotoxin-I_{N}-HIS) in E. coli are shown in FIG. 23. In particular, ratios of spliced products and unspliced precursors were determined by immunoblot analysis of bacterial lysates using antibodies against a engineered epitope (6×HIS epitope tag).

In addition, replacement strategies analogous to those described above can be performed, using recombinant DNA techniques to replace designated cysteine residues within the cyclizing peptides with codons encoding other amino acids, and the resulting peptides are then tested for activity. One advantage associated with the use of intein splicing to generate cyclic conotoxin-derived peptides is the economy and high throughput of this approach, which permits the systematic replacement of one or both sets of cysteines in the α-conotoxin GI peptide with amino acids that feature non-disulfide forming side chains described above. Therefore, all possible permutations can be readily generated and tested. The use of recombinant DNA techniques to generate all possible permutations facilitates this.

One class of conotides of the invention is derived from the α and αA-conotoxins which are competitive antagonists of nicotinic acetylcholine receptors (nAChRs). There are two distinct families of nAChRs in vertebrates; muscle-type nAChRs are located at skeletal neuromuscular junctions, while neuronal nAChRs are located in the central and peripheral nervous system. Muscle and neuronal nAChRs consist of five homologous subunits arranged around a central pore. α-conotoxins with subtype specificity for muscular and neuronal nAChRs have been characterized. α-conotoxins EI, GI, MI and SI target the muscular nAChRs, which are conjunction with glutamic acid or aspartic acid substitution of the partnering cysteine residue at position 13 to create a conotide with improved activity. The opposite configuration, in which an acidic residue is substituted for cysteine at position 3 and a basic residue replaces the cysteine at residue 13 can also be used. This substitution strategy is applied to either disulfide-forming cysteine pair (e.g. 3-13 and 2-7 pairs in the α-conotoxin GI peptide example) either singly (one at a time) or in combination (one or more disulfide bond forming cysteine pairs at a time).

The conotides described above are then tested by comparing the conotide against the conotoxin it was derived from (e.g., synthesizing α-conotoxin GI in cyclic and non-cyclic form) for binding to, e.g., nAChRs, e.g., using the T. californica electric organ assay, as recognized in the art.

### Example 5

### Methods and Compositions for Producing Conotides Using Intein-Mediated Protein Splicing

In this example, methods and compositions for using intein-mediated protein splicing to generate cyclic conotoxin-derived peptides (“conotides”), are described.

Briefly, conotoxin coding sequences are expressed as a fusion protein between the I_{C} and I_{N} intein splicing domains. Upon translation of the I_{C}-conotoxin-I_{N} fusion protein or conotide, followed by spontaneous intein-mediated protein splicing, a cyclic peptide product composed of conotoxin residues is produced, along with the I_{C} and I_{N} side products. This can be carried out in a variety of contexts, including in vitro translation of the I_{C}-conotoxin-I_{N} fusion protein, as well as expression of the I_{C}-conotoxin-I_{N} fusion protein in bacteria, yeast, insect cells (e.g. recombinant baculoviral expression), or in mammalian cells (e.g., CHO cells).

For expression of conotoxin-derived peptides (conotides) in mammalian cells, conotoxin derived coding sequences are inserted between the I_{C} and I_{N} elements of the pIMPS vector. Expression of the I_{C}-conotoxin-I_{N} peptide results in intein splicing and production of the cyclized conotide. A preferred class of conotides are derived from a class of conotoxins that do not depend on extensive post-translational modification. The α and αA-conotoxins meet this requirement as well as other members of conotoxin families that have an amidated C-terminus such that there are no post-translational modifications in the “small loop” structure believed to be the binding site.

In generation of cyclized conotoxin-derived peptides (conotides) such as the α-conotoxin GI peptide with the naturally occurring sequence ECCNPACGRHYSC, gene cassettes express the following cyclic permutations of this sequence. Scanning of cyclic permutations allows the examination of several configurations of the splicing reaction and insert, and allows selection of the most efficient intein splicing form for conotide production. (However, it is structural elements of conotoxin-derived backbones, allows for the ability to screen random, structurally constrained conotide libraries for unique functions. By expressing and screening these libraries inside the cell, a broad range of conotides capable of altering a cell phenotype can be identified.

Several types of libraries are contemplated. For example, libraries based on naturally occurring conotoxin families can be generated by inserting DNA fragments shown below into a vector suitable for their expression and translation.

| | |
|---|---|
| α-Conotoxin-Based Random Peptide Libraries: | |
| ATG (NNN)₁₋₂ TGT TGT NNN CCC GCC TGT GGG (NNN)₄ TGT (NNN)₀₋₄ TGA | (SEQ ID NOS: 34, 35) |
| M X C C X P A C G X C X * | |
| μ-Conotoxin-Based Random Peptide Libraries: | |
| ATG (NNN)₂ TGT TGT (NNN)₂ CCC CCC TGT (NNN)₄ TGT NNN CCC (NNN)₂ TGT TGT NNN TGA | (SEQ ID NOS: 36, 37) |
| M X C C X P P C X C X P X C C X * | |
| ω-Conotoxin-Based Random Peptide Libraries: | |
| ATG TGT (NNN)₆ CTT (NNN)₅₋₆ TGT TGT (NNN)₂₋₃ TGT (NNN)₄₋₆ TGT (NNN)₀₋₆ TGA | (SEQ ID NOS: 38, 39) |
| M C X P X C C X C X C X * | |

In the libraries described above, the cysteine residues are retained. However, additional libraries can be made that incorporate the substitution strategies described above. Namely, codons encoding charged amino acids capable of forming salt bridges can be used to replace cysteine partners. For example, such a library based on an α-conotoxin can be composed using the following oligonucleotide inserts:

| | |
|---|---|
| α-Conotoxin-Based Random | |
| Non-Cysteine/Salt Bridged Peptide Libraries: | |
| ATG (NNN)₁₋₂ GAN AAA NNN CCC GCC AAA GGG (NNN)₄ GAN (NNN)₀₋₄ TGA | (SEQ ID NOS: 40, 41) |
| AGA AGA | |
| M X E/D R/K X P A R/K G X E/D X * | |

Analogous libraries based on the μ- and ω-conotoxins incorporate complementarily charged residues at respective cysteine partners, in such a way that the salt bridges formed can fulfill the structural roles of the endogenous disulfide bonds in these conotoxin backbone families.

Stacking of aromatic rings is another strategy for the replacement of structural cysteines in this format. Again, using the α-conotoxin backbone as an example:

| | |
|---|---|
| α-Conotoxin-Based Random | |
| Peptide Libraries Constraining Stacked Aromatics: | |
| ATG (NNN)₁₋₂ TGG TGT NNN CCC GCC TGG GGG (NNN)₄ TGT (NNN)₀₋₄ TGA | (SEQ ID NOS: 42, 43) |
| M X W C X P A W G X C X * | |
| ATG (NNN)₁₋₂ TGT TGG NNN CCC GCC TGT GGG (NNN)₄ TGG (NNN)₀₋₄ TGA | (SEQ ID NOS: 44, 45) |
| M X C W X P A C G X W X * | |
| ATG (NNN)₁₋₂ TGG TGG NNN CCC GCC TGG GGG (NNN)₄ TGG (NNN)₀₋₄ TGA | (SEQ ID NOS: 46, 47) |
| M X W W X P A W G X W X * | |

In these libraries, codons encoding one or more pairs of aromatic amino acids replace one or more cysteine pairs in the backbone. Although tryptophan (W) is shown in this example, all possible combinations of aromatic amino acids (i.e. tryptophan, tyrosine, or phenylalanine) are also contemplated. In addition, the same strategy is applicable to libraries based on the μ- and ω-conotoxin family backbones.

In one embodiment, the receptor binding activity and specificity of the identified conotide(s) is compared to a naturally occurring conotoxin. In another embodiment, the conotide is assayed for its ability to alter a cell phenotype, e.g., when expressed in a cell. This process identifies conotides with desired biochemical or biological activities, along with improvement of other chemical or physiological properties. In addition, this process allows the systematic derivation of a structure/activity relationship profile that informs the transformation of other conotoxins into conotides, or the design of random peptide library schemes with improved properties, as described below.

### Example 7

### Use of Intein-Mediated Splicing to Generate and Phenotypically Screen Conotide Libraries

The substitution strategies used for the libraries described above describes the generation of linear peptide libraries that can be expressed inside cells for phenotypic screening. This invention also encompasses the generation and screening of libraries derived from the conotoxin backbones, but that are spontaneously cyclized intracellularly through intein mediated splicing.

In all examples below, the nucleotide sequences shown are inserted in frame with both upstream Ic sequences and downstream In sequences, resulting in the production of a self-processing fusion protein composed of Ic-Conotoxin Derived Library Peptide-In. In using the pIMPS vector, oligonucleotide primers are designed such that an Mfe I compatible cloning site is present upstream of the library insert, and a Hind III compatible cloning site downstream of the library insert (see FIG. 17).

In this approach, intein-mediated cyclization facilitates the ability to eliminate structurally important cysteines, and, using the substitution strategies described above (i.e., amino acid substitutions that lead to salt bridge formation, stacking of aromatic amino acid residues, etc.), leads to stable, structurally constrained and organized cyclic amino acids that feature privileged conotoxin-like backbone structures, but that do not rely upon cysteines for their structural integrity and function, and that are present intracellularly, where they have access to a milieu of novel targets. Although only libraries based on themes of the α-conotoxin backbone are shown herein, the invention also encompasses these strategies applied to the generation and screening of libraries based on the μ- and ω-conotoxin family backbone, using, for example, the basic formula I_{C}-conotide insert (e.g., SEQ ID NOS: 34-47)-I_{N}.

In summary, libraries generated in this manner use conotoxin peptide backbone structures, further informed and improved through identification and systematic replacement of critical but potentially labile or unstable secondary structures (i.e. cysteine replacement and formation of a cyclically contiguous amide backbone) to screen and identify conotides that modulate categories of targets known to be susceptible to the activities of naturally occurring conotoxin peptides, as well as novel classes of targets, including intracellular or other novel targets for which a phenotypic or functional activity in mammalian cells is known. This includes all varieties of signal transduction cascades, metabolic regulatory systems, susceptibility to infection or pathogenesis of viruses, bacteria, or toxins thereof.

### Example 8

### Methods and Compositions for Producing Aptamer Libraries Fused to Hedgehog for Sterol Conjugation and Membrane Localization

In this example, methods and compositions for producing peptide aptamers that undergo hedgehog polypeptide-mediated sterol conjugation are described.

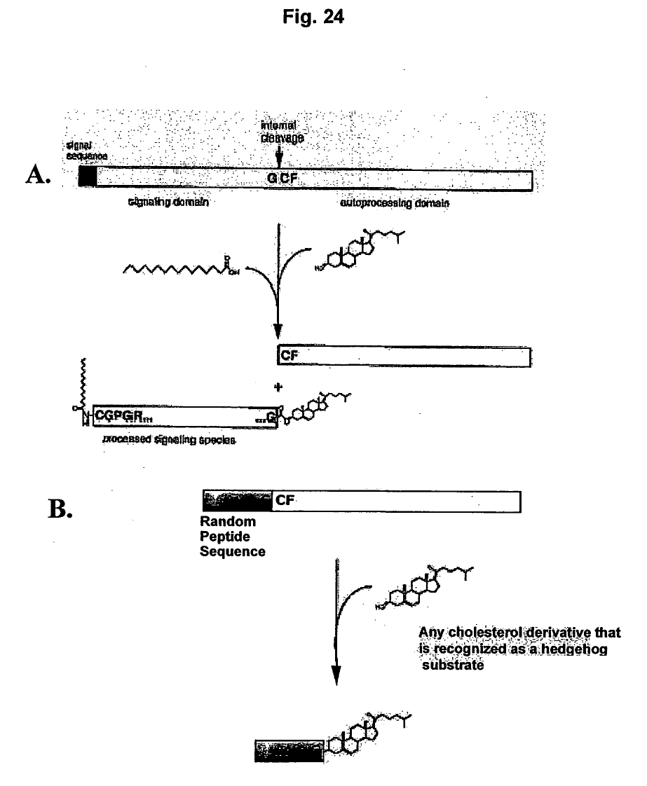

The aptamer libraries of the invention can be engineered to take advantage of the sterol conjugation domain of the hedgehog polypeptide. Previous experiments examining the mechanism of hedgehog autocleavage have demonstrated that although the integrity of the C-terminal region of the hedgehog protein (i.e., Hh-C domain) is critical for function, most of the N-terminal domain (ie., Hh-N domain) is dispensable (FIG. 24A).

Accordingly, aptamer peptide libraries can be constructed in which the coding sequences for random peptides are fused in-frame with the essential C-terminal region of hedgehog polypeptides such that hedgehog splicing results in the covalent modification of cholesterol with random peptides (see FIG. 24B). Moreover, this esterification reaction can occur with cholesterol derivatives and other sterols, e.g., 7-dehydrocholesterol, 5-androsten-3β-ol (Mann and Beachy, 2000)). Accordingly, this same library of fusion polypeptides can be used to produce a library of peptides conjugated to any cholesterol or sterol molecule in which such splicing can occur.

The utility of such a library is that cholesterol-conjugated peptides can be localized to the cell membrane. Accordingly, directed localization of these peptide/sterol conjugates can facilitate the identification of molecules that act through targets localized at the cell surface (e.g. ion channels or membrane transporters, signal transduction receptors, or effectors of receptor mediated signal transduction). Alternatively, cholesterol or sterol-conjugated peptides can be discovered that affect various aspects of cholesterol metabolism, both with respect to lipid metabolism and homeostasis (i.e. LDL, HDL, etc.) and synthesis or activities of various cholesterol derived sterol hormones. In other specific aspects of the invention, libraries affecting phenotypes associated with the functions of cellular proteins with cholesterol sensing activity (e.g., SCAP, HMG CoA reductase, patched, dispatched) or cholesterol or sterol metabolism (e.g. SREBP, ACAT1, ACAT2, CETP), ABC1 mediated cholesterol transport, or other intracellular cholesterol trafficking or localization, can be used.

In addition, multiple functions have been ascribed to lipid rafts and caveolae, cholesterol-rich sub-compartments of the plasma membrane (reviewed in Galbiati et al., 2001). These include, but are not limited to, modulation of the activities of signaling proteins, including oncogenes (e.g. ras, raf, rac, rho) and G-protein coupled receptors or their effectors, ion channels, receptor kinases or effectors of their activity, protein import or export, intracellular lipid or protein compartmentalization, as well as viral entry or egress.

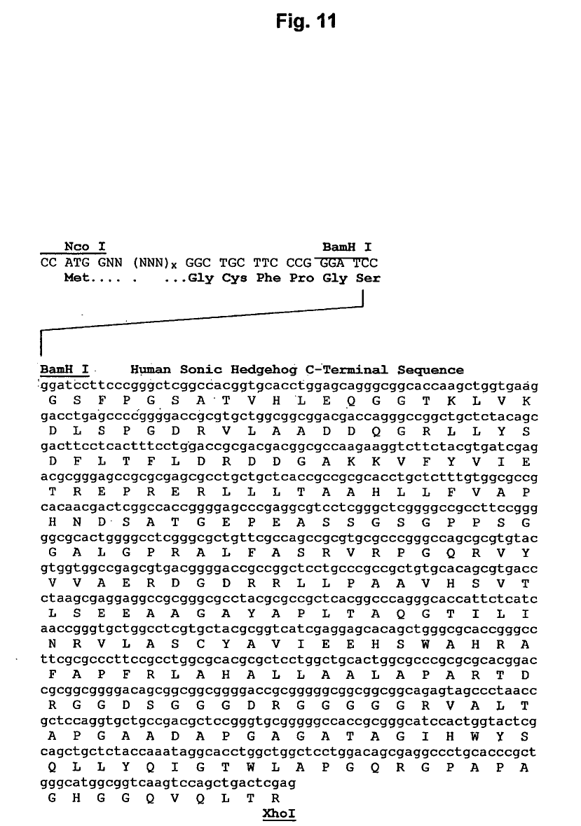

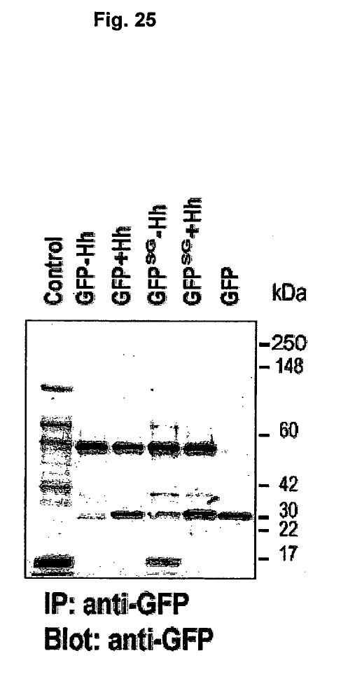

An exemplary aptamer/hedgehog expression vector was constructed as follows. Briefly, a cDNA fragment encoding the carboxy terminus of human sonic hedgehog (SEQ ID NO: 48) was cloned into the BamH I and Xho I sites of the pMFG expression vector (other species of hedgehog polypeptide can also be used). The resultant hedgehog cleavage and corresponding cloning sites for the aptamer sequence is illustrated in FIG. 11 and the sequence of the essential hedgehog polypeptide region (human sonic hedgehog) is provided in SEQ ID NO: 49. Since splicing activity of this minimal C-terminal hedgehog domain coupled to heterologous upstream peptide or protein sequences has not been previously examined or demonstrated, a proof-of-concept experiment was performed in which a gene cassette encoding the GFP protein was cloned upstream of and in frame with the C-terminal hedgehog domain. This plasmid, encoding the fusion protein in the pMFG vector, was transfected into mammalian cells. Cleavage and processing was assessed by immunoblot analysis of transfected cells (FIG. 25). These results demonstrate the presence of an intracellular cleaved GFP in cells expressing a splicing-competent GFP-Shh fusion protein. Importantly, spliced GFP protein in the hedgehog-processing samples had slightly slower mobility in SDS-PAGE gel analysis than an unmodified intracellularly expressed GFP protein, consistent with the covalent attachment of cholesterol to the cleaved GFP product of the intracellularly processed GFP-Shh fusion protein.

In a manner analogous to the introduction of GFP coding sequences upstream of the C-Shh coding sequences in the experiment described above, oligonucleotides encoding random amino acid sequences can be inserted, and expressed intracellularly to yield spontaneously processed cholesterol-ligated peptide libraries. The length of this random coding region is predetermined in the design of the oligonucleotides. In addition, specific features of peptides or proteins may be incorporated into the design of such libraries and their coding sequences. These include such elements as SH2 or SH3 domains, or libraries that fix certain elements of these domains (i.e. one or more amino acid residues identified as consensus sequences), while randomizing the remaining positions. Such an approach allows for the exploration of additional sequences that improve the binding, activity, or specificity against a given target, while in other cases, such a strategy expands the range of potential targets that can be addressed using a given library. The foregoing steps result in a retroviral library in which sequences encoding a random peptide of determined length is expressed in frame with the C-terminal domain of a selected hedgehog polypeptide. The species of origin or the exact identity of the hedgehog polypeptide is not critical, as long as the polypeptide selected retains the ability to spontaneously auto-process and conjugate a sterol (e.g., to a peptide).

The oligonucleotide library can then be packaged into retroviruses by co-transfection into EcoPac cells with the pVSV-G vector as described herein. The resulting retroviral library can be used to transduce a plurality of test cells and phenotypic selection is carried out. Cells expressing a peptide-hedgehog fusion that is processed to form a cholesterol-peptide conjugate that manifested a desired phenotypic can then be identified.

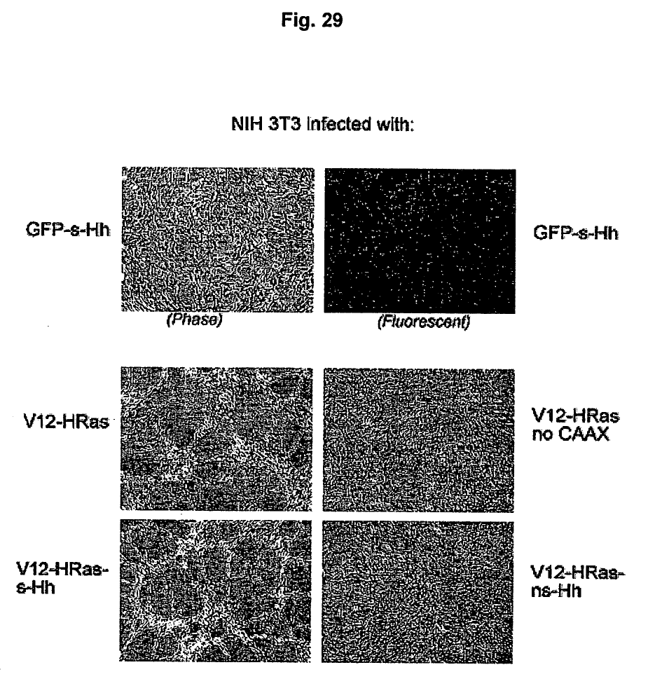

As a proof of principle for this approach, the green fluorescent protein (GFP) was fused with the catalytic domain of hedgehog (human) having either a functional or non-functional cleavage site (e.g., the functional cleavage site has the sequence Ser-Gly-Gly-Cys-Phe whereas the nonfunctional cleavage site has the sequence Ser-Gly-Gly-Ala-Phe where the absence of a Cys destroys the cleavage site) and cloned into a retroviral expression vector. Constructs were packaged into ecotropic retroviruses and correct protein expression and effective cleavage and sterol conjugation, based on altered mobility, was confirmed by immunoblot. Supernatants of the transfected cells bearing retroviral particles containing the constructs were also used to infect NIH 3T3 cells using standard techniques. Cells were then washed and examined for phenotypic changes. Cells infected by virus containing a GFP hedgehog fusion fluoresced showing that the GFP sterol conjugated by hedgehog was functionally active. (FIG. 29).

To confirm that biologically functional sterol conjugations can be made, cells having an activated Ras, Ras lacking a membrane targeting domain (CAAX), and Ras lacking a membrane targeting domain but fused to the catalytic domain of hedgehog, were examined for their ability to transform cells using the above approach. the activated form of Ras (V12Ras) is strongly transforming whereas Ras lacking a membrane targeting domain (CAAX) is not. However, Ras lacking the membrane targeting domain but fused to hedgehog was sterol conjugated, targeted to a membrane, and rescued for its transforming activity (see FIG. 29). Controls indicated that this restoration of a biologically functional sterol conjugation, depended on a functional hedgehog protein. Further, immunoblotting of cholesterol rich membrane fractions confirmed that activated Ras and Ras sterol conjugated by hedgehog where correctly targeted to the membrane whereas Ras lacking a membrane targeting domain (CAAX) and Ras lacking a membrane targeting domain (CAAX) fused to a nonfunctional hedgehog protein where not.

An additional use of the library is as an inducible system, in which although the peptide-hedgehog fusion protein is expressed intracellularly, expression is initially carried out in “cholesterol starved” cells. Such starvation is sufficient to halt self-processing of the native hedgehog precursor protein (Guy et al., PNAS, 97:7307-12 (2000)). In the absence of cholesterol, the cells retain viability, but autoprocessing stops. Thus, at a desired time or circumstance, cholesterol, a specified sterol, or a mixture of sterols is then introduced to the media and the cell population expressing the peptide-hedgehog library initiates processing, sterol conjugation, and membrane targeting of the fused polypeptide, e.g., peptide aptamer.

In another application of the inducible system, prokaryotic cells may be used because they have the advantage of lacking any potential substrate sterols. Accordingly, the sterol conjugation of a given polypeptide by hedgehog can be precisely controlled and will only occur upon addition of an exogenous sterol (e.g., cholesterol). Other advantages for sterol conjugating peptides in prokaryotic cells include the fast generation time of prokaryotic cells, the ease of their manipulation, their ability to produced large amounts of heterologous proteins or peptides, and their ability to be arrayed (e.g. 96 well or other multiwell plate formats) to form vast libraries in either the conjugated or unconjugated form for various screening applications as described herein.

A typical application using prokaryotic cells can be performed as follows. Briefly, prokaryotic expression vectors (e.g., comprising a pET vector backbone) encoding proteins or peptides fused to a sterol conjugation domain (e.g., hedgehog ) are introduced into a suitable strain of prokaryotic cells (e.g., BL21 cells, see e.g., Studier et al., Methods Enzymol. 185:60-89 (1990) and Ausabel et al., Current Protocols in Molecular Biology, John Wiley, Pub., (2002) for an extended list of suitable prokaryotic expression hosts). Then, upon exposure to a sterol, the sterol conjugation domain carries out an autoprocessing reaction in the cells that results in the covalent conjugation of a sterol molecule (e.g., cholesterol) to the protein or peptide (e.g. a random peptide aptamer). In one approach, a single sterol is added to a culture of prokaryotic cells expressing the random peptide coding sequences expressed in frame with the sterol conjugation domain. This results in a library of peptides all conjugated to an identical sterol. In another approach, two or more sterols are added to a mixed culture (or separate cultures) such that a library of peptides fused to different sterols is obtained.

Typically, the resultant sterol conjugated peptide libraries are isolated by either arranging for their secretion into the media or by lysing the cells expressing the peptides. The resultant sterol conjugated peptides are then purified to a desired homogeneity for further applications using standard techniques (e.g., by engineering an immunotag in the peptide, e.g., HIS tag for capture using metal chelating resins or cysteine capture using thiol reactive capture).

### Example 9

### Methods and Compositions for Increasing Expression of Retroviral Libraries and Efficiency of Phenotypic Screening in Transduced Mammalian Cells

In this example, methods and compositions for producing high expression retroviral vectors for aptamer peptide expression and screening, are described.

Typically, upon entry of a retrovirus into a host mammalian cell, a DNA copy of the RNA genome is generated by reverse transcription, and this proviral form is stably integrated into the host cell genome. Frequently, however, an integrated retrovirus may fail to express (be transcriptionally inert). Moreover, it is well established that within a given clonal cell population of transduced cells, although a given proviral integrant may be transcriptionally active early after integration, gene expression may decrease significantly as the cell passes through subsequent cell divisions or differentiation.

The invention provides for the use of chromatin insulator elements to optimize the establishment of populations of cells expressing retroviral libraries early after transduction and proviral integration, as well as their utility in perpetuating expression throughout the growth of these cells, and the use of these cells in phenotypic selection assays either in tissue culture or in animals. The chromatin insulator is a genetic element that is engineered into the retroviral library vector itself, and serves to augment and perpetuate expression of the retroviral library inserts at each site of integration within a plurality of cells, and throughout the continued growth or differentiation of the cells. This is of particular use in perpetuated expression in transgenic mouse models, or in experimental systems where expression in myeloid or lymphoid cell systems is desired.

In brief, a fragment such as that described in Reitman, et al. (Mol. Cell. Biol., 10:2774-2786 (1990)), which comprises the element provided in SEQ ID NO: 50, is introduced at or near the LTR region of the retroviral vector as described by, e.g., Emery et al., (Proc. Natl. Acad. Sci. USA 97: 9150-9155 (2000)) and in U.S. Pat. No. 5,610,053.

The modified retroviral vector encoding the insulator element(s) is then used to express one or more aptamers in the test cell line without the disadvantage of poor expression (e.g., transcriptional silencing). Accordingly, the inherent complexity and sensitivity of the aptamer library screen is improved such that every recipient test cell receives an actively expressed aptamer whose potential alteration of the cell phenotype can be detected.

### Example 10

### Methods and Compositions for Producing Cells with High Transduction Efficiency for High Throughput Screening of Retroviral Libraries

In this example, the genetic engineering of a cell to express an ecotropic receptor for improved infectivity by a retroviral vector, is described.

The receptor for ecotropic murine retroviruses has been identified and cloned. The receptor protein, known as ATRC-1, is a membrane spanning cationic amino acid transporter. Although expressed ubiquitously in mammalian cells, differences exist between the human and mouse proteins that render the murine version of the gene specific for murine retroviruses; ecotropic murine retroviruses do not infect human cells.

The present invention provides for the use of the ATRC-1 receptor in two general ways: (i) to improve infectivity of mouse or rat cells by ecotropic murine retroviruses by establishing mouse cell lines or transgenic animals overexpressing the receptor; and (ii) expansion of the host range of retroviral libraries in ecotropic murine retrovirus vectors by expression of the mouse receptor in human cells or cells of other species not normally infected by ecotropic murine retroviruses.

Accordingly, efficiency is improved in terms of the number of transduced and integrated viral genomes per infected cell, and/or by increasing the number of individual cells in which retroviral transduction occurs. Moreover, the phenotypic screening of random peptides or RNA aptamers in mammalian cells requires very high efficiency in transduction of the retroviral library containing the sequences to be screened. This frequently a limiting step in the ability to carry out such screens in a manner that allows expression and examination of large libraries in a way that ensures as thorough a survey as possible of the complexity of the library in each phenotypic screen. Still further, another advantage is that multiple retroviruses can be used to transduce and integrate into a given cell, effectively allowing for the screening of multiple candidates aptamers per cell.

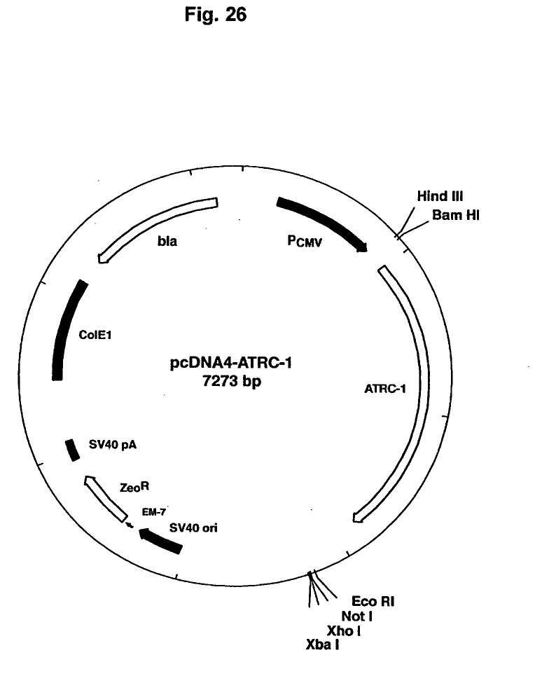

In brief, the selected cell line to be used for phenotypic screening is transfected with a vector encoding the ATRC-1 receptor (Accession No. M26687; FIG. 26; SEQ ID NO: 51-52; see also, Sommerfelt, J. Gen. Virol. 80: 3049-3064 (1999); Albritton et al. Cell 57: 659-666 (1989); Albritton et al., J. Virol. 67: 2091-2096 (1993)). Preferably, the receptor is stably integrated into the genome of the test cell, is stably expressed after many cell passages, and has no phenotypic consequences for the cell. The receptor is introduced into the cell using standard nucleic acid transfer techniques and the vector used to express the receptor can be, e.g., a retroviral vector or recombinant expression vector.

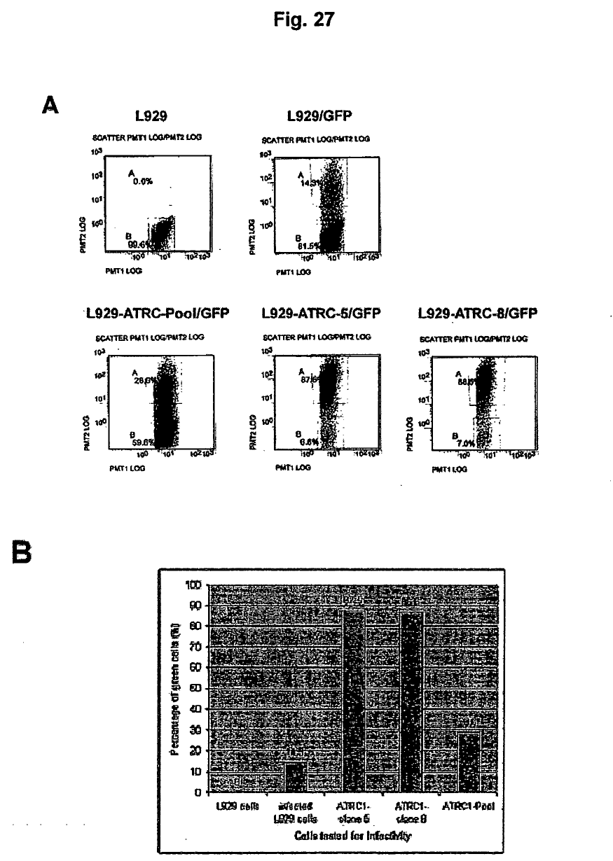

Use of ATRC-1 to improve the transduction efficiency of ecotropic retrovirus in mouse cell lines is demonstrated in FIG. 27. In this experiment, mouse L929 cells, which undergo low transduction efficiency using ecotropic retrovirus, were transfected with a plasmid expressing the ATRC-1 protein using the CMV enhancer/promoter. The expression vector also contains a zeomycin resistance selectable marker. Following transfection of the ATRC-1 expression plasmid and selection for zeomycin resistant cells, resistant cells were collected, and are referred to as the “pools” in this figure. Selection of clonal cells derived from this mixture were also generated. These clonal lines, along with the cell pool and mock transfected cells were tested for transduction efficiency using a recombinant ecotropic virus encoding GFP. Following transduction (and selection using zeomycin), efficiency was assessed both with respect to the percentage of cells expressing GFP, and the relative brightness of GFP expressing cells as compared top a control transduced with vector alone using FACS (FIG. 27A-B). The latter parameter is an approximate indication of the average number of transduction events per cell genome (i.e. copy number per cell). L929-ATRC-5 and L929-ATRC-8 are clonal cell lines that were highly transducible subclones selected from the original transfection pool of zeomycin resistant transfectants. L929-ATRC-Pool cells represent a mixed population of cells that were transfected and with pcDNA4-ATRC-1 and selected for zeomycin resistance.

Control experiments in which human 293T cells that are not transducible using ecotropic retrovirus, demonstrate that stable expression of mouse ATRC-1 in these cells results in a population of cells that are transducible with almost 100% efficiency using a recombinant ecotropic retrovirus expressing GFP.

### Example 11

### Methods for Screening Peptide Aptamers Capable of Modulating Apoptosis in a Human Myeloid Leukemia

In this example, methods for screening peptide aptamers capable of modulating apoptosis in human myeloid leukemia HL-60 cells are described.

HL-60 cells are a well characterized human myeloid leukemia cell line in which apoptosis is inducible. These cells also grow in suspension, and their apoptotic response to multiple stimuli has been characterized (reviewed in Darzynkiewicz et al., 1992). Accordingly, in one approach, cells pre-cleared of spontaneously apoptotic cells are contacted with an aptamer library encoded in an expressible form on a plasmid, preferably a retrovirally derived vector, that can efficiently enter the cell and express a particular aptamer. Then, HL-60 cells in which apoptosis has been induced by an expressed aptamer, are identified. Any art recognized FACS or panning strategies can be used for detecting the approximate 100-1000 apoptotic cells per 10 million cells that represent a desirable level of sensitivity and selectivity required for the apoptotic aptamer screen.

After selection of apoptotic cells is achieved, the aptamers that induce apoptosis in HL-60 cells, are then tested for their ability cause apoptosis in non-cancerous human cells, such as primary fibroblasts, with a preferably result being that the selected aptamer works preferentially in only cancerous cells.

Each aptamer identified in the HL-60 screen is then tested in a diverse panel of human cancer cell lines. This aspect of the invention allows for the identification of a set of aptamers sufficient to induce apoptosis in as many different types of cancer cells as possible.

In the initial screening steps, apoptotic cells are identified using APOPTEST™ or an analogous method, both before and after induction of aptamer expression. Briefly, in contrast to TUNEL staining methods for identifying apoptotic cells, which identify apoptosis by end-labeling DNA fragments that arise late, APOPTEST™ stains cells early in apoptosis, and does not require fixation of the cell sample (Martin et al., 1994). This method uses an annexin V antibody to detect cell membrane re-configuration that is characteristic of cells undergoing apoptosis. Apoptotic cells stained in this manner can then sorted either by fluorescence activated cell sorting (FACS), or by adhesion and panning using immobilized annexin V antibodies.

Retroviral sequences in cells identified and segregated in this manner can be amplified by PCR, and the aptamers can be recloned and validated. In later rounds of aptamer re-screening and validation, at the point where aptamers are being re-tested individually, other methods of apoptosis can be employed as a counterscreen. These include such methods as TUNEL staining or propidium iodide staining. This is necessary to ensure that the selected phenotype is in fact apoptosis, and not an aptamer-induced alteration in membrane metabolism. Validated apoptotic aptamers are then expressed in a variety of non-cancerous cells and other cancer cell lines to determine their specificity and range of action. Aptamers can be identified that do not induce apoptosis in non-tumor cells, although they can also be evaluated for their ability to induce apoptosis in other cell lines, as described herein.

The invention also encompasses screens that can be conducted for identification of aptamers that augment the sensitivity of cancer cells to radiation or cancer chemotherapeutic agents. In addition, aptamers can act synergistically with the apoptotic response to these agents, either by impacting the same pathways, or by targeting novel but parallel cellular responses to these agents. In each case, aptamer library expression can be induced in a population of transduced cells, followed by treatment with an agent known to induce apoptosis, but at a dose below the threshold for this response.

### Example 12

### Methods for Screening Peptide Aptamers Capable of Modulating a Cellular Phenotype Using a Peptide Library that can be Inducibly Expressed or Which can be Functionally Activated

In this example, methods for screening peptide aptamers capable of modulating apoptosis in human myeloid leukemia HL-60 cells, whereby the peptide aptamers can be inducibly expressed, are described.

This assay is performed in a manner similar to the assay described above, with the advantage that the expression of the peptide aptamer is inducible. In this variation of the screen, it is important to discriminate between spontaneous apoptosis and aptamer induced apoptosis by inducing aptamer expression at a given time, and identifying the cells that have undergone apoptosis subsequent to this induction. A number of commercially available transcriptional regulation systems exist that regulate transcription upon exposure or removal of specific compounds (e.g. ecdysone or tetracycline). However, these systems require multiple time consuming modifications of the host cell, including integration and stable expression of several plasmids harboring the numerous components of the system. Expression of these various required components can be unstable and unreliable. Moreover, such reagents are not amenable to use in high-throughput systems (i.e. generation and screening of large libraries): populations of cells in which appropriate regulation is achieved cannot be derived en masse. Instead, for expression of any given protein using these systems, it is often necessary to screen numerous individual clones to identify a given clonal cell population in which desired regulation of expression (i.e. acceptable shut-off and induction) has been achieved.

One approach to achieving wholesale and reliable regulation of the peptide within each individual transduced cell is through the use of regulatory elements that are integrated into the retroviral vector itself, and do not require the co-expression and activity of secondary activator or repressor proteins. One such approach is exemplified in a manner described by Werstuck and Green (1998), in which an RNA element that binds an exogenous ligand is introduced into the 5′ UTR of a gene. When cells expressing RNA containing this regulatory element are exposed to this cell-permeable ligand (in this case a Hoechst dye), translation of a downstream reporter is drastically reduced. By incorporating such a feature in the 5′UTR of retroviral vectors encoding random peptide libraries in mammalian cells, such a regulatory element allows for coordinated and controlled repression/induction of library expression within a population of transduced cells.

Alternatively, instead of regulating expression of the expressed library proteins per se, activity of the proteins or precursors proteins may be coordinately regulated. For example, in use of the intein mediated cyclization technology described above, “crippled” inteins that retain critical splicing residues, but do not undergo autonomous Ic-In association sufficient to sponsor efficient spontaneous splicing, may be fused to protein sequences known to carry out inducible protein-protein interaction through the activity of extraneous ligands. For example, the FK506 binding domain of FKBP12, when expressed within both the Ic and In elements of a crippled Ic-pep-In precursor, can undergo inducible splicing upon introduction of FK1012, a dimeric form of FK506 that has been shown to mediate such interactions in other systems (Rollins et al., 2000). Other protein/ligand systems that can be used include, but are not limited to, the growth hormone-inducible system as described (Guo et al., Science, 288:2042-2045 (2000).

Accordingly, an inducible retroviral peptide aptamer library can be used to transduce ten to 100 million cells growing in suspension. For example, HL-60 cells can be used since they have robust growth in suspension, and have well characterized responses to various apoptotic stimuli, which can be useful in examining the apoptotic phenotypes. Apoptosis resulting from activity of peptides encoded by the transduced library can be distinguished from spontaneous apoptosis by presorting and removing cells undergoing apoptosis prior to induction of library expression or activity, either by washing cells to remove the translational-repressing RNA binding agent (allowing translation of the aptamer peptide) or by introduction of a dimerization-inducing ligand that prompts intein splicing and production of library peptide cyclization. (In the latter case, the system has the additional advantage of selecting for peptides that have activity only when they are present in a cyclic form.) Cells are then re-sorted to identify cells in which apoptosis occurred following aptamer expression.

### Example 13

### Methods for Screening Peptide Aptamers Capable of Modulating Apoptosis Using Growth Factor Dependent Cells

In this example, methods for screening peptide aptamers capable of modulating apoptosis using the BaF3 cell line are described.

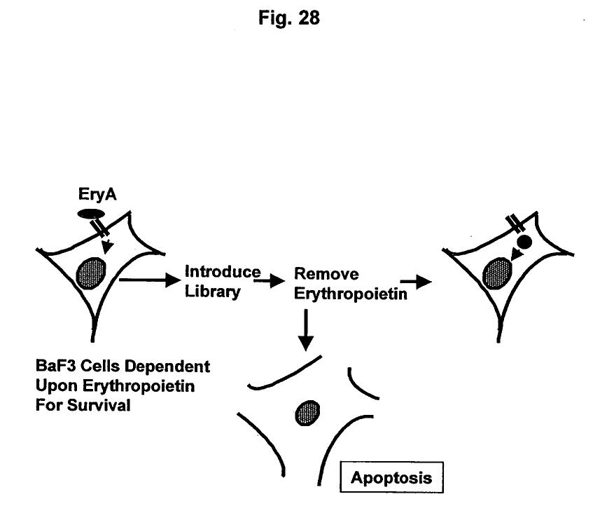

To identify and catalog aptamers that carry out signaling via various signal transduction pathways, a well established growth factor dependent (IL-3) BaF3 cell-based assay system can be employed (see, e.g., Kitamura et al., 1995). BaF/3 cells require continuous stimulation of certain pathways in order to forgo apoptosis: removal or attenuation of some certain stimulatory signals or signaling cascades, results in BaF/3 apoptosis. For example, withdrawal of IL-3 induces apoptosis of BaF/3 cells. Erythropoietin signaling can substitute for that of IL-3 in BaF/3 cells engineered to express the erythropoietin receptor (EpoR). In these cells, the erythropoietin mediated signal cascade is sufficient to rescue cells from apoptotic death in the absence of IL-3 growth factor. In this screen, a retroviral library is used to infect mouse BaF/3 cells, which normally undergo apoptosis upon withdrawal of IL-3. BaF/3 cells stably expressing EpoR are used to screen and identify retrovirus encoded peptide aptamers that abrogate apoptosis of BaF3 cells in response to withdrawal of erythropoietin. (Growth and library selection are carried out in the absence of IL-2.) A schematic summary of this selection strategy is shown in FIG. 28. Aptamer coding sequences from the surviving cells are then amplified by PCR, recloned into a mammalian expression vector, and re-screened by reintroduction into naive BaF3 cells, and the process is repeated iteratively.

This assay system allows for the discovery of active aptamers, and phenotypes associated with aptamer expression can be easily deconvoluted. For example, this abrogation of the BaF/3 apoptotic phenotype is not observed in the presence of activated forms of the abl oncogene. Accordingly, expression of aptamers that stimulate Abl kinase activity, or the activity of appropriate downstream components that signal transduction pathway, can result in cell survival; aptamers that cause survival of BaF3 cells through stimulation of Abl can be identified by the susceptibility of these cells to Novartis ST1571, a specific inhibitor of the Abl kinase that is in clinical use for the treatment of some leukemias (Carroll et al., 1997). The survival phenotype of these cells can the be reversed by the ST157 1 kinase inhibitor, whereas aptamer mediators of Jak2 or STAT5 activity can be identified by examination of these proteins and their activities in aptamer expressing cells (Nosaka et al., 1999; McCubrey et al., 2000).

In another selection scheme, a library of random peptides is transduced into a population of myeloid or lymphoid precursor cells from mice that are defective for development or differentiation. Aptamers can then be expressed ectopically in erythropoietin receptor knockout mice. These mice are deficient for erythropoiesis, which is reconstituted by activation of the Abl kinase (Ghaffari et al., 1999).

Aptamers that specifically substitute for erythropoietic signals can be used as lead compounds for the development of small molecule drugs for therapeutic use in the treatment of anemia in kidney dialysis and cytotoxic chemotherapeutic treatments.

Further, if desired, aptamers identified in the BaF3 anti-apoptotic screen can also be tested for their effects on hematopoietic stem cell development and differentiation.

Accordingly, this system allows for the identification of active aptamers capable of modulating apoptosis and a method for understanding their mode of action.

### Example 14

### Methods for Screening Peptide Aptamers Capable of Modulating Intracellular Signaling Cascades

In this example, methods for screening peptide aptamers capable of modulating intracellular signaling cascades are described.

In general, signaling cascades refer to networks of molecular interactions and activities through which an environmental or developmental stimulus is received and interpreted by a cell. This carefully orchestrated molecular response is a designated sequence of events that ultimately leads to an alteration in cellular metabolism or function. G protein coupled receptors (GPCRs) are a large and growing gene family of transmembrane proteins. To date, over 1000 GPCRs have been cloned. These receptors are classified both by the types of extracellular signals to which they respond (e.g. photons, odors, ions, monoanines, or peptides), and by the particular trimeric G protein effector complex that mediates intracellular transmission and amplification of receptor signaling. Ligand mediated signaling through these receptors results in a broad spectrum of responses.

The present invention provides aptamer libraries that can be screened for members that modulate a cellular response analogous to that resulting from ligand engagement by a given receptor, or that inhibit such a response, in the presence of ligand. The invention also provides methods for screening libraries to identify aptamers that abrogate, attenuate, or alter the specificity of receptor mediated signaling that occurs upon binding of the receptor by a cognate (endogenous or exogenous) ligand. Receptor tyrosine kinase signaling cascades, as well as receptor mediated signaling cascades that mediate signaling through src family kinases, are other pathways that can also be targeted using this system.

### Example 15

### Methods for Screening Peptide Aptamers Capable of Modulating Protein Transport and Trafficking

In this example, methods for screening peptide aptamers capable of modulating protein transport and trafficking are described.

The present invention provides methods for identifying aptamers that can affect trafficking of specific proteins to the cell surface. This has particular utility in cases where misdirection of proteins is associated with disease. Aptamer libraries can be introduced into clonal cell lines stably expressing the mislocalized protein. Cells expressing aptamers that affect membrane localization of the desired protein can be identified by staining non-permeabilized cells with a specifically reactive antibody. Positively staining cells can then be physically separated by either fluorescence activated cell sorting (FACS) or other appropriate art recognized techniques. Aptamers can be identified that correct the mislocalization or induce the relocalization of any protein at the cell surface, including various receptors and channels, antigens, or proteins involved in the immune response. In the latter case, involving modulation of antigen presentation in an immune response, an aptamer can either augment an immune response to specific infections, especially in immunocompromised individuals, or attenuate certain aspects of immunity that can be beneficial in autoimmune syndromes or conditions.

An example of a particular application of the foregoing methods can be for the development of therapeutics for hemochromatosis, an autosomal recessive disorder in which approximately 95% of the non-functional protein encoded by mutant alleles is no longer directed to the cell surface (Waheed et al., 1997). This leads to an alteration of iron transport in certain intestinal cells of individuals homozygous for this mutant allele, and chronic accumulation of iron in the serum to levels that lead to long-term organ toxicity. Hemochromatosis is, in fact, the most common hereditary disorder among Caucasians, affecting up to one in every two hundred Americans, and leading to liver, kidney, and other organ failure, the etiology of which, had not been previously appreciated. Analogous screens can be carried out to identify modulators of transport of disease-associated alleles of the cystic fibrosis (CF) chloride channel protein, which, unlike its non-disease associated counterparts, is not efficiently transported to the surface of the cell.

Still another application of the foregoing methods is the following. Many viruses, including HIV infected T cells in which the HIV nef gene product down-regulates MHC-mediated antigen presentation (reviewed in Collins and Baltimore, 1999), various herpesviruses, including cytomegalovirus (CMV) (del Val et al., 1997; Kleijnen et al., 1997), and papillomavirus, actively suppress antigen presentation as a means of eluding or evading immune recognition and response (reviewed in McFadden and Kane, 1994). Clonal cell lines either chronically infected by these viruses, or constitutively expressing virus encoded proteins that affect these functions involving protein transport or trafficking, can be infected with a retroviral aptamer library, and cells in which antigen presentation was augmented or reconstituted as measured by, e.g., FACS, can be scored using the subsequent steps described above, and a candidate aptamer that can modulate the pathway can be identified.

In other embodiments, cells can be identified for their ability to express or not express specific markers characteristic of certain types of immune cells (e.g. markers associated with T cell subtypes).

### Example 16

### Methods for Screening Peptide Aptamers Capable of Modulating Cell Adhesion

In this example, methods for screening peptide aptamers capable of modulating cell adhesion are described.

Cell adhesion is an important element of development and the immune response. Cell surface adhesion molecules function both as mediators of physical association between cells and as important sensors and transmitters of intracellular signals. For example, the integrin proteins of leukocytes and neutrophils serve as adhesive molecules that immobilize these cells to sites of localized immune or allergic response, and in turn, trigger intracellular responses upon adherence (i.e. degranulation). The cell sorting protocols described herein can be easily adapted for panning, in which aptamers that can modulate (e.g., induce certain adhesive properties in a cell) are identified.

### Example 17

### Methods for Screening Peptide Aptamers Capable of Modulating Membrane Transport

In this example, methods for screening peptide aptamers capable of modulating membrane transport are described.

Membrane transport of ions and other ligands plays an important role in many physiological processes and disease states. For example, ATP cassette transport proteins have a wide variety of functions, including mediating efflux of drugs. The human multiple drug receptor membrane protein (MDR) presents a significant clinical problem in patients undergoing cancer chemotherapy, by efficiently pumping cancer therapy drugs out of cancer cells, thereby limiting their efficacy. Other members of this family are associated with peroxisomes, mutant forms of which are associated with disease, including adenoleukodystrophy. Art recognized dyes exist that can be used to identify cells in which these transporters are unable to mediate efflux of certain types of compounds.

Accordingly, these techniques can be used to screen retroviral peptide aptamer libraries when used to infect clonal cell lines that endogenously overexpress an MDR or other ATP cassette protein, or in which this gene or a mutant form is stably expressed. Cells expressing aptamers capable of increasing dye retention in these cells can be sorted, and the sequence of the encoded aptamer can be determined.

### Example 18

### Methods for Screening Peptide Aptamers Capable of Modulating Cell Motility and Chemotaxis

In this example, methods for screening peptide aptamers capable of modulating cell motility and chemotaxis are described.

Neutrophils are among the first leukocytic cells to migrate into tissues in response to invading pathogens or other initiators of inflammatory injury. One of the first steps of neutrophil involvement in acute inflammation is chemotaxis, directed movement toward chemotactic agents, such as complement fragments (C5a), cytokines (IL-8), leukotrienes, and bacteria-derived peptides such as formyl-methionine-leucine-phenylalanine (fMLP). Inhibition of this chemoattractive response is an effective means of abrogating inflammation, especially in diseases like asthma and the chronic inflammation associated with cystic fibrosis.

Accordingly, cell lines stably expressing a chemotactic receptor can be infected with a retroviral peptide aptamer library, and migration toward a specific chemoattractant can be measured using art recognized transwell assays in which the cells are placed in an upper chamber, and the chemoattractant is placed in a lower chamber. After a time sufficient for transmigration of the chemotactic cells across the chamber barrier, cells remaining in the upper chambers can be pooled, grown out, and re-assayed serially until a population of truly non-responsive cells is identified.

Thus, using this approach, which can be readily adapted to a high throughput format, aptamers that can modulate cell motility and/or chemotaxis can be identified.

### Example 19

### Methods for Screening Peptide Aptamers Capable of Modulating Viral Resistance

In this example, methods for screening peptide aptamers capable of modulating viral resistance, are described.

In particular, using split inteins, a random cyclic peptide library was engineered by placing random oligonucleotides between the coding sequences of I_{C} and I_{N} intein domains and the library was then expressed in cells. The peptides expressed in cells are then determined if they perturb the course of a viral infection. As a model system, a virus was used that promotes certain cell death and belongs to a family that has been implicated as being pathogenic in humans. Specifically, the encephalomyocarditis virus (EMCV), a cardiovirus of the picornavirus family, was used. EMCV is a cardiovirus of the picornavirus family. Its single, positive-stranded RNA genome encodes a large open reading frame that, upon infection, is translated into a single polyprotein. This polyprotein is subsequently processed via a series of proteolysis events into its component proteins. EMCV can infect a wild-range of cells with a preference for rodents. Infections by EMCV induce a massive programmed cell death, also named apoptosis. The screen is performed, to test whether cyclic peptides can interfere with any viral associated processes, e.g., viral infection: membrane attachment, reverse translation of the genomic RNA, inhibition of antiviral responses of the cells, inhibition of the apoptotic machinery or production of new viral particles.

The assay was performed as described above and number of colonies resistant to viral mediated apoptosis were enumerated. Significantly more colonies are observed on plates containing cells transduced with the retroviral peptide library than in the control plates (GFP retrovirus) indicating that peptide aptamers that confer viral resistance are present in the library. The peptide aptamers are recovered from the cells as described, e.g., in Example 1.

Publications Referred to in the Specification

Aravind, et al., Trends Biochem Sci. 24:47-53 (1999)
Balis, Oncologist 3:V (1998)
Baserga, Cell 79:927-930 (1994)
Blind, Proc. Natl. Acad. Sci. USA. 96: 3606-3610 (1999)
Boland, et al., Proc Natl Acad Sci USA. 96:14675-14677 (1999)
Burns et al., Proc Natl Acad Sci USA. 90: 8033-8037 (1993)
Carroll et al., Blood 90:4947-52 (1997)
Cohen et al., Proc. Natl. Acad. Sci. USA. 95:14272-14277 (1998)
Colas et al., Nature 380:548-550 (1996)
Collins et al., Immunol. Rev. 168:65-74 (1999)
Curt, Oncologist 1: II-III (1996)
Darzynkiewicz et al., Cytometry 13:795-808 (1992)
del Val et al., J. Exp. Med. 76:729-38 (1992)
Evan et al., Cell 69:119-128 (1994)
Fisher et al., Science 286:2507-2510 (1999)
Gerszten et al., Proc. Natl. Acad. Sci. USA. 96:8567-8572 (1999)
Ghaffari et al., Proc. Natl. Acad. Sci. USA. 96:13186-131901(1999)
Gui et al., Science 288: 2042-2045 (2000)
Hanrahan et al., Cell 100:57-70 (2000).
Harrington et al., Curr. Opin. Genet. Dev. 4:120-129 (1994).
Ikushima et al., Proc Natl Acad Sci USA. 94:2609-2614 (1997).
Kitamura et al., Proc Natl Acad Sci USA. 92:9146-9150 (1995).
Klausner et al., AACR-NCI-EORTC International Conference. Washington, D.C., (Nov. 16, 1999)
Kolonin et al., Proc. Natl. Acad. Sci. USA. 95:14266-14271 (1998).
Li et al., Curr. Opin. Cell Biol. 11:261-266 (1999)
Martin et al., J. Exp. Med. 182:1545-1556 (1994)
McCubrey et al., Leukemia 14:9-21 (2000)
McFadden et al., Adv. Cancer Res. 63: 117-209 (1994)
McLafferty et al., Gene 128:29-36 (1993)
Monks et al., J. Nat. Cancer Inst. 83:757-766 (1991)
Norman et al., Science 285:591-595 (1999)
Nosaka et al., EMBO J. 18:4754-4765 (1999)
Osborne et al., Curr. Opin. Chem. Biol. 1:5-9 (1997)
Pear et al., Proc Natl Acad Sci USA. 90:8392-8396 (1993)
Perler et al., NAR 25:1087-1093 (1997)
Polyak et al., Nature 389:300-305 (1997)
Schlessinger, Cell 100:293-296 (2000)
Schlesinger et al., Frontiers in Bioscience 3:1181-1186 (1998)
Sellers et al., Journal Clin. Invest. 104:1655-1661 (1999)
Shaw et al., Proc Natl Acad Sci USA. 89: 4495-9 (1992)
Shi et al., Proc. Natl. Acad. Sci. USA. 96:10033-10038 (1999)
Shih et al., Cell 29:161-169(1982)
Tan et al., J. Am. Chem Soc. 120:8565-8566 (1998)
Tao et al., Proc. Natl. Acad. Sci. USA. 97:783-786 (2000)
Yonish-Rouach et al., Nature 352:345-7 (1991)
Verma et al., Nature 389:239-242 (1997)
Vojtek et al., J. Biol. Chem. 273:19925-19928 (1998)
Waheed et al., Proc. Natl. Acad. Sci. USA. 94: 12384-12389 (1997)
Zujewski et al., J. Clin. Oncol. 18:927 (2000)
   Equivalents

Those skilled in the art will recognize, or be able to ascertain using no more than routine experimentation, many equivalents to the specific embodiments of the invention described herein. Such equivalents are intended to be encompassed by the following claims.

1. A method of identifying a peptide aptamer capable of modifying a cell phenotype comprising:
a) contacting a first sample of cells with a library of expressible nucleic acid sequences encoding random peptide aptamers linked to a fusion moiety;
b) selecting at least one cell having an altered phenotype compared to the phenotype of the cell prior to the contacting step (a); and
c) identifying one or more peptide aptamers expressed in the selected cell.

2. The method of claim 1 further comprising amplifying the nucleic acid sequences encoding the one or more peptide aptamers identified in step c) and repeating steps a)-c) using the amplified nucleic sequences as the library of expressible nucleic acid sequences specified in step a).

3. The method of claim 2, wherein steps a)-c) are repeated two or more times.

4. The method of claim 1 further comprising genetically modifying the first sample of cells to express a receptor not naturally expressed by the cells.

5. The method of claim 4, wherein the receptor is an ecotropic receptor.

6. The method of claim 1 wherein a sample of cells expresses an ecotropic receptor.

7. The method of claim 1, further comprising expressing the one or more peptide aptamers identified in step c) in a second sample of cells, and identifying a change in the phenotype of the second sample of cells.

8. The method of claim 7, wherein the cell type of the first and second sample of cells differs.

9. The method of claim 7, further comprising contacting the second sample of cells with a pathogen before or after expression of the one or more peptide aptamers in the second sample of cells.

10. The method of claim 7, further comprising contacting the second sample of cells with an agent which stimulates signal transduction before or after expressing the one or more peptide aptamers in the second sample of cells.

11. The method of claim 7, wherein the change in the phenotype of the cells comprises a change in the expression levels of an endogenous gene or gene products associated with said phenotype.

12. The method of claim 7, wherein the change in the phenotype of the cells is selected from the group consisting of a change in levels of apoptosis, signal transduction, protein trafficking, cell adhesion, membrane transport, cell motility, viral resistance, metabolic state, or cellular differentiation, as compared to a control cell.

13. The method of claim 7, wherein said cellular differentiation is selected from the group consisting of the induction of hair growth, promotion of hair growth, hair follicle differentiation, hair follicle differentiation development, melanogenesis, hair shaft elongation, skin cell differentiation, and a shift in the hair growth cycle from telogen (resting stage) to anagen (growing stage).

14. The method of claim 4, wherein the cells of the second sample contain a reporter gene.

15. The method of claim 14, wherein the change in phenotype of the cells comprises a change in the expression levels of the reporter gene.

16. The method of claim 1, wherein the altered phenotype is associated with a change in levels of apoptosis, signal transduction, protein trafficking, cell adhesion, membrane transport, cell motility, viral resistance metabolic state, or cellular differentiation as compared to a control cell.

17. The method of claim 16, wherein the change in levels of apoptosis is measured using immunohistochemistry.

18. The method of claim 1, wherein the altered phenotype is associated with a change in levels of signal transduction.

19. The method of claim 1, wherein the change in levels of signal transduction is primarily mediated by a receptor or downstream effectors of a receptor.

20. The method of claim 1, wherein the change in levels of signal transduction is primarily mediated by an exogenous receptor or downstream effectors of an exogenous receptor.

21-204. (Cancelled)


SEQUENCE LISTING (346 sequences)

SEQ ID NO: 1 - protein, 9 aa, Artificial Sequence
  Description of Artificial Sequence Synthetic peptide
SXXXGPXXX

SEQ ID NO: 2 - DNA, 6870 bp, Artificial Sequence
  Description of Artificial Sequence Synthetic construct
aagctagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatggcaatattcctgttaccaaagctagtataaataaaaatagataaacgtggaaattacttagagtttctgtcattaacgtttccttcctcagttgacaacataaatgcgctgctgagaagccagtttgcatctgtcaggatcaatttcccattatgccagtcatattaattactagtcaattagttgatttttatttttgacatatacatgtgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgcgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatggttaaagttatcggtcgtcgttccctcggagtgcaaagaatatttgatattggtcttccccaagaccataattttctgctagccaatggggcgatcgcccacaattgttttaacggatcccccgggctgcaggaattcgatatcaagctttggcaccgaaattttaaccgttgagtacggcccattgcccattggcaaaattgtgagtgaagaaattaattgttctgtgtacagtgttgatccagaagggagagtttacacccaggcgatcgcccaatggcatgaccggggagagcaggaagtattggaatatgaattggaagatggttcagtaatccgagctacctctgaccaccgctttttaaccaccgattatcaactgttggcgatcgaagaaatttttgctaggcaactggacttgttgactttagaaaatattaagcaaactgaagaagctcttgacaaccatcgtcttccctttccattacttgacgctgggacaattaaataagtcgacctcgagatcccccagatcccgcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 3 - DNA, 1391 bp, Artificial Sequence
  Description of Artificial Sequence Synthetic construct
tctagagccgccatggttaaagttatcggtcgtcgttccctcggagtgcaaagaatatttgatattggtcttccccaagaccataattttctgctagccaatggggcgatcgcccacaattgttttaacggatcccatcatcatcatcatcatggtatggctagcatgactggtggacagcaaatgggtcgggatctgtacgacgatgacgataaggtacctaggatcgaacaaaaactcatctcagaagaggatctgggcatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccggcacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacggatcccccgggctgcaggaattcgatatcaagcttgattgcctcagttttggcaccgaaattttaaccgttgagtacggcccattgcccattggcaaaattgtgagtgaagaaattaattgttctgtgtacagtgttgatccagaagggagagtttacacccaggcgatcgcccaatggcatgaccggggagagcaggaagtattggaatatgaattggaagatggttcagtaatccgagctacctctgaccaccgatttttaaccacgattatcaactgttggcgatcgaagaaatttttgctaggcaactggacttgttgactttagaaaatattaagcaaactgaagaagctcttgacaaccatcgtcttccctttccattacttgacgctgggacaattaaataagtcgac

SEQ ID NO: 4 - protein, 461 aa, Artificial Sequence
  Description of Artificial Sequence Synthetic construct
SRAAMVKVIGRRSLGVQRIFDIGLPQDHNFLLANGAIAHNCFNGSHHHHHHGMASMTGGQQMGRDLYDDDDKVPRIEQKLISEEDLGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYGSPGLQEFDIKLDCLSFGTEILTVEYGPLPIGKIVSEEINCSVYSVDPEGRVYTQAIAQWHDRGEQEVLEYELEDGSVIRATSDHRFLTTIINCWRSKKFLLGNWTCLKILSKLKKLLTTIVFPFHYLTLGQLNKS

SEQ ID NO: 5 - DNA, 11 bp, Artificial Sequence
  Description of Artificial Sequence Synthetic construct
tgyytaagctt

SEQ ID NO: 6 - DNA, 15 bp, Artificial Sequence
  Description of Artificial Sequence Synthetic construct
aattsynnntgyyta

SEQ ID NO: 7 - DNA, 108 bp, Artificial Sequence
  Description of Artificial Sequence Synthetic construct
gaattctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgcttaagctt

SEQ ID NO: 8 - protein, 35 aa, Artificial Sequence
  Description of Artificial Sequence Synthetic construct
NSXXXXXXXXXXXXXXXXXXXXXXXXXXXXXXCLS

SEQ ID NO: 9 - DNA, 46 bp, Artificial Sequence
  Description of Artificial Sequence Synthetic primer
gccgtctagagccgccatggttaaagttatcggtcgtcgttccctc

SEQ ID NO: 10 - DNA, 46 bp, Artificial Sequence
  Description of Artificial Sequence Synthetic primer
gctagtcgacttatttaattgtcccagcgtcaagtaatggaaaggg

SEQ ID NO: 11 - protein, 10 aa, Artificial Sequence
  Description of Artificial Sequence Synthetic peptide
EQKLISEEDL

SEQ ID NO: 12 - DNA, 36 bp, Artificial Sequence
  Description of Artificial Sequence Synthetic construct
aatnnnnnnnnnnnnggnccnnnnnnnnnnnnnnnn

SEQ ID NO: 13 - DNA, 36 bp, Artificial Sequence
  Description of Artificial Sequence Synthetic construct
agctnnnnnnnnnnnnnnnnggnccnnnnnnnnnnn

SEQ ID NO: 14 - protein, 10 aa, Artificial Sequence
  Description of Artificial Sequence Synthetic construct
XXXXGPXXXX

SEQ ID NO: 15 - DNA, 36 bp, Artificial Sequence
  Description of Artificial Sequence Synthetic construct
aatttgttcctgtacgggccctggaaagacttgtta

SEQ ID NO: 16 - DNA, 36 bp, Artificial Sequence
  Description of Artificial Sequence Synthetic construct
agcttaacaagtctttccagggcccgtacaggaaca

SEQ ID NO: 17 - protein, 10 aa, Artificial Sequence
  Description of Artificial Sequence Synthetic construct
LFLYGPWKDL

SEQ ID NO: 18 - DNA, 36 bp, Artificial Sequence
  Description of Artificial Sequence Synthetic construct
aatttgttcctgtacgccgcctggaaagacttgtta

SEQ ID NO: 19 - DNA, 38 bp, Artificial Sequence
  Description of Artificial Sequence Synthetic construct
aaagcttaacaagtctttccaggcggcgtacaggaaca

SEQ ID NO: 20 - protein, 11 aa, Artificial Sequence
  Description of Artificial Sequence Synthetic peptide
LFLYXXWKDLL

SEQ ID NO: 21 - DNA, 36 bp, Artificial Sequence
  Description of Artificial Sequence Synthetic construct
aattcgttcctgtacgccgcctggaaagactgctta

SEQ ID NO: 22 - DNA, 36 bp, Artificial Sequence
  Description of Artificial Sequence Synthetic construct
agcttaagcagtctttccaggcggcgtacaggaacg

SEQ ID NO: 23 - protein, 11 aa, Artificial Sequence
  Description of Artificial Sequence Synthetic peptide
SFLYXXWKDCL

SEQ ID NO: 24 - DNA, 1251 bp, Artificial Sequence
  Description of Artificial Sequence Synthetic construct
atggttaaagttatcggtcgtcgttccctcggagtgcaaagaatatttgatattggtcttccccaagaccataattttctgctagccaatggggcgatcgcccacaattctgaagaggacttgggaggcggtgccatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccggcacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggtggcggtgaacagaagctcatctgcttaagctttggcaccgaaattttaaccgttgagtacggcccattgcccattggcaaaattgtgagtgaagaaattaattgttctgtgtacagtgttgatccagaagggagagtttacacccaggcgatcgcccaatggcatgaccggggagagcaggaagtattggaatatgaattggaagatggttcagtaatccgagctacctctgaccaccgctttttaaccaccgattatcaactgttggcgatcgaagaaatttttgctaggcaactggacttgttgactttagaaaatattaagcaaactgaagaagctcttgacaaccatcgtcttccctttccattacttgacgctgggacaattaaataa

SEQ ID NO: 25 - protein, 416 aa, Artificial Sequence
  Description of Artificial Sequence Synthetic construct
MVKVIGRRSLGVQRIFDIGLPQDHNFLLANGAIAHNSEEDLGGGAMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYKGGGGEQKLICLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 26 - protein, 13 aa, Artificial Sequence
  Description of Artificial Sequence Synthetic peptide
ECCNPACGRHYSC

SEQ ID NO: 27 - DNA, 45 bp, Artificial Sequence
  Description of Artificial Sequence Synthetic construct
tgctgcaacccggcgtgcggtcgtcattatagctgcggtagcgaa

SEQ ID NO: 28 - protein, 15 aa, Artificial Sequence
  Description of Artificial Sequence Synthetic construct
CCNPACGRHYSCGSE

SEQ ID NO: 29 - DNA, 45 bp, Artificial Sequence
  Description of Artificial Sequence Synthetic construct
tgcggtcgtcattatagctgcggtagcgaatgctgcaacccggcg

SEQ ID NO: 30 - protein, 15 aa, Artificial Sequence
  Description of Artificial Sequence Synthetic construct
CGRHYSCGSECCNPA

SEQ ID NO: 31 - DNA, 45 bp, Artificial Sequence
  Description of Artificial Sequence Synthetic construct
tcgtgcggtagcgaatgctgcaacccggcgtgcggtcgtcattat

SEQ ID NO: 32 - protein, 15 aa, Artificial Sequence
  Description of Artificial Sequence Synthetic construct
SCGSECCNPACGRHY

SEQ ID NO: 33 - protein, 13 aa, Artificial Sequence
  Description of Artificial Sequence Synthetic construct
ICCNPACGPLYSC

SEQ ID NO: 34 - DNA, 60 bp, Artificial Sequence
  Description of Artificial Sequence Synthetic construct
atgnnnnnntgttgtnnncccgcctgtgggnnnnnnnnnnnntgtnnnnnnnnnnnntga

SEQ ID NO: 35 - protein, 19 aa, Artificial Sequence
  MOD_RES (2)..(3): Variable amino acid
MXXCCXPACGXXXXCXXXX

SEQ ID NO: 36 - DNA, 69 bp, Artificial Sequence
  Description of Artificial Sequence Synthetic construct
atgnnnnnntgttgtnnnnnncccccctgtnnnnnnnnnnnntgtnnncccnnnnnntgttgtnnntga

SEQ ID NO: 37 - protein, 22 aa, Artificial Sequence
  MOD_RES (2)..(3): Variable amino acid
MXXCCXXPPCXXXXCXPXXCCX

SEQ ID NO: 38 - DNA, 105 bp, Artificial Sequence
  Description of Artificial Sequence Synthetic construct
atgtgtnnnnnnnnnnnnnnnnnncttnnnnnnnnnnnnnnnnnntgttgtnnnnnnnnntgtnnnnnnnnnnnnnnnnnntgtnnnnnnnnnnnnnnnnnntga

SEQ ID NO: 39 - protein, 34 aa, Artificial Sequence
  MOD_RES (3)..(8): Variable amino acid
MCXXXXXXLXXXXXXCCXXXCXXXXXXCXXXXXX

SEQ ID NO: 40 - DNA, 60 bp, Artificial Sequence
  Description of Artificial Sequence Synthetic construct
atgnnnnnnganarannncccgccaaagggnnnnnnnnnnnnrrnnnnnnnnnnnnntga

SEQ ID NO: 41 - protein, 19 aa, Artificial Sequence
  MOD_RES (2)..(2): Variable amino acid
MXXXXXPAKGXXXXXXXXX

SEQ ID NO: 42 - DNA, 60 bp, Artificial Sequence
  Description of Artificial Sequence Synthetic construct
atgnnnnnntggtgtnnncccgcctgggggnnnnnnnnnnnntgtnnnnnnnnnnnntga

SEQ ID NO: 43 - protein, 19 aa, Artificial Sequence
  MOD_RES (2)..(3): Variable amino acid
MXXWCXPAWGXXXXCXXXX

SEQ ID NO: 44 - DNA, 60 bp, Artificial Sequence
  Description of Artificial Sequence Synthetic construct
atgnnnnnntgttggnnncccgcctgtgggnnnnnnnnnnnntggnnnnnnnnnnnntga

SEQ ID NO: 45 - protein, 19 aa, Artificial Sequence
  MOD_RES (2)..(3): Variable amino acid
MXXCWXPACGXXXXWXXXX

SEQ ID NO: 46 - DNA, 60 bp, Artificial Sequence
  Description of Artificial Sequence Synthetic construct
atgnnnnnntggtggnnncccgcctgggggnnnnnnnnnnnntggnnnnnnnnnnnntga

SEQ ID NO: 47 - protein, 19 aa, Artificial Sequence
  MOD_RES (2)..(3): Variable amino acid
MXXWWXPAWGXXXXWXXXX

SEQ ID NO: 48 - DNA, 805 bp, Homo sapiens
  CDS (1)..(804)
ggatccttcccggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctggctcctggacagcgaggccctgcacccgctgggcatggcggtcaagtccagctgactcgag

SEQ ID NO: 49 - protein, 268 aa, Homo sapiens
GSFPATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLAPGQRGPAPAGHGGQVQLTR

SEQ ID NO: 50 - DNA, 242 bp, Gallus gallus
agggacagcccccccccaaagcccccagggatgtaattacgtccctcccccgctagggcagcagcgagccgcccggggctccggtccggtccggcgctcccccgcatccccgagccggcagcgtgcggggacagcccgggcacggggaaggtggcacgggatcgctttcctctgaacgcttctcgctgctctttgagcctgcagacacctggggggatacggggaaaaaagctttaggctga

SEQ ID NO: 51 - DNA, 1869 bp, Mus musculus
atgggctgcaaaaacctgctcggtctgggccagcagatgctgcgccggaaggtggtggactgcagccgggaggagagccggctgtcccgctgcctcaacacctatgacctggtagctcttggggtgggcagcaccttgggcgctggtgtctatgtcctagccggtgccgtggcccgtgaaaatgctggccctgccatcgtcatctccttcttgattgctgctctcgcctccgtgctggccggcctgtgctacggcgagtttggtgcccgtgtccccaagacgggctcagcctacctctacagctacgtgacggtgggggagctttgggccttcatcactggctggaacctgattctctcctacatcatcggtacttcaagcgtggcaagagcctggagtgcgacttttgacgagctgataggcaagcccatcggagagttctcacgtcagcacatggccctgaatgctcctggggtgctggcccaaaccccggacatatttgctgtgattataattatcatcttaacaggactgttaactcttggcgtgaaggagtcagccatggtcaacaaaattttcacctgtatcaatgtcctggtcttgtgcttcatcgtggtgtccgggttcgtgaaaggctccattaaaaactggcagctcacggagaaaaatttctcctgtaacaacaacgacacaaacgtgaaatacggtgagggagggtttatgccctttggattctctggtgtcctgtcaggggcagcgacctgcttttatgccttcgtgggctttgactgcatcgccaccacaggggaagaagtcaagaacccccagaaggccattcctgtgggcatcgtggcgtccctcctcatttgcttcatagcgtactttggcgtgtccgccgctctcacgctcatgatgccttacttctgcctggacatcgacagcccgctgcctggtgccttcaagcaccagggctgggaagaagctaagtacgcagtggccattggctctctctgcgcactttccaccagtctcctaggctccatgtttcccatgccccgagttatctatgccatggctgaagatggactactgtttaaatttttggccaaaatcaacaataggaccaaaacacccgtaatcgccactgtgacctcaggcgccattgctgctgtgatggccttcctctttgaactgaaggacctggtggacctcatgtccattggcactctcctggcttactctttggtggctgcctgtgttttggtcttacggtaccagccagaacaacctaatctggtataccagatggccagaaccaccgaggagctagatcgagtagatcagaatgagctggtcagtgccagtgaatcacagacaggctttttaccggtagccgagaagttttctctgaaatccatcctctcacccaagaacgtggagccctccaaattctcagggctaattgtgaacatttcagccggcctcctagccgctcttatcatcaccgtgtgcattgtggccgtgcttggaagagaggccctggccgaagggacactgtgggcagtctttgtaatgacagggtcagtcctcctctgcatgctggtgacaggcatcatctggagacagcctgagagcaagaccaagctctcatttaaggtaccctttgtccccgtacttcctgtcttgagcatcttcgtgaacatctatctcatgatgcagctggaccagggcacgtgggtccggtttgcagtgtggatgctgataggtttcaccatctatttcggttatgggatctggcacagtgaggaagcgtccctggctgctggccaggcaaagactcctgacagcaacttggaccagtgcaaatga

SEQ ID NO: 52 - protein, 622 aa, Mus musculus
MGCKNLLGLGQQMLRRKVVDCSREESRLSRCLNTYDLVALGVGSTLGAGVYVLAGAVARENAGPAIVISFLIAALASVLAGLCYGEFGARVPKTGSAYLYSYVTVGELWAFITGWNLILSYIIGTSSVARAWSATFDELIGKPIGEFSRQHMALNAPGVLAQTPDIFAVIIIIILTGLLTLGVKESAMVNKIFTCINVLVLCFIVVSGFVKGSIKNWQLTEKNFSCNNNDTNVKYGEGGFMPFGFSGVLSGAATCFYAFVGFDCIATTGEEVKNPQKAIPVGIVASLLICFIAYFGVSAALTLMMPYFCLDIDSPLPGAFKHQGWEEAKYAVAIGSLCALSTSLLGSMFPMPRVIYAMAEDGLLFKFLAKINNRTKTPVIATVTSGAIAAVMAFLFELKDLVDLMSIGTLLAYSLVAACVLVLRYQPEQPNLVYQMARTTEELDRVDQNELVSASESQTGFLPVAEKFSLKSILSPKNVEPSKFSGLIVNISAGLLAALIITVCIVAVLGREALAEGTLWAVFVMTGSVLLCMLVTGIIWRQPESKTKLSFKVPFVPVLPVLSIFVNIYLMMQLDQGTWVRFAVWMLIGFTIYFGYGIWHSEEASLAAGQAKTPDSNLDQCK

SEQ ID NO: 53 - protein, 6 aa, Artificial Sequence
  Description of Artificial Sequence Synthetic peptide
GCFPGS

SEQ ID NO: 54 - protein, 6 aa, Artificial Sequence
  Description of Artificial Sequence Synthetic 6xHis tag
HHHHHH

SEQ ID NO: 55 - protein, 5 aa, Artificial Sequence
  Description of Artificial Sequence Synthetic peptide
SGGCF

SEQ ID NO: 56 - protein, 5 aa, Artificial Sequence
  Description of Artificial Sequence Synthetic peptide
SGGAF

SEQ ID NO: 57 - protein, 13 aa, Artificial
  Description of Artificial Sequence Synthetic peptide
CFAKGTNVLMADG

SEQ ID NO: 58 - protein, 13 aa, Artificial
  Description of Artificial Sequence Synthetic peptide
CFTKGTQVMMADG

SEQ ID NO: 59 - protein, 13 aa, Artificial
  Description of Artificial Sequence Synthetic peptide
CLTSDHTVLTTRG

SEQ ID NO: 60 - protein, 13 aa, Artificial
  Description of Artificial Sequence Synthetic peptide
CISKFSHIMWSHV

SEQ ID NO: 61 - protein, 13 aa, Artificial
  Description of Artificial Sequence Synthetic peptide
CISGDSLISLAST

SEQ ID NO: 62 - protein, 13 aa, Artificial
  Description of Artificial Sequence Synthetic peptide
CLAEGTRIFDPVT

SEQ ID NO: 63 - protein, 13 aa, Artificial
  Description of Artificial Sequence Synthetic peptide
CMNYSTRVTLADG

SEQ ID NO: 64 - protein, 13 aa, Artificial
  Description of Artificial Sequence Synthetic peptide
CLTADARINVKGK

SEQ ID NO: 65 - protein, 13 aa, Artificial
  Description of Artificial Sequence Synthetic peptide
CVSGNSLVRLLFG

SEQ ID NO: 66 - protein, 13 aa, Artificial
  Description of Artificial Sequence Synthetic peptide
CVTGDALVRLPFG

SEQ ID NO: 67 - protein, 13 aa, Artificial
  Description of Artificial Sequence Synthetic peptide
CVTGDALVRLPFG

SEQ ID NO: 68 - protein, 13 aa, Artificial
  Description of Artificial Sequence Synthetic peptide
CLTGDALVRLPFG

SEQ ID NO: 69 - protein, 13 aa, Artificial
  Description of Artificial Sequence Synthetic peptide
CITGDALVALPEG

SEQ ID NO: 70 - protein, 13 aa, Artificial
  Description of Artificial Sequence Synthetic peptide
SILPNEWLPIIEN

SEQ ID NO: 71 - protein, 13 aa, Artificial
  Description of Artificial Sequence Synthetic peptide
SILPEEWVPLIKN

SEQ ID NO: 72 - protein, 13 aa, Artificial
  Description of Artificial Sequence Synthetic peptide
SILPEEWLPVLEE

SEQ ID NO: 73 - protein, 13 aa, Artificial
  Description of Artificial Sequence Synthetic peptide
SILPDEYLTIIEE

SEQ ID NO: 74 - protein, 13 aa, Artificial
  Description of Artificial Sequence Synthetic peptide
SVSGESEIIIRQN

SEQ ID NO: 75 - protein, 13 aa, Artificial
  Description of Artificial Sequence Synthetic peptide
CHPADTKVVVKGK

SEQ ID NO: 76 - protein, 13 aa, Artificial
  Description of Artificial Sequence Synthetic peptide
CHPKGTKVVVKGK

SEQ ID NO: 77 - protein, 13 aa, Artificial
  Description of Artificial Sequence Synthetic peptide
CVPPDTLLILENG

SEQ ID NO: 78 - protein, 13 aa, Artificial
  Description of Artificial Sequence Synthetic peptide
CLTSNSKILTDDG

SEQ ID NO: 79 - protein, 13 aa, Artificial
  Description of Artificial Sequence Synthetic peptide
CLMPHEKVLTEYG

SEQ ID NO: 80 - protein, 13 aa, Artificial
  Description of Artificial Sequence Synthetic peptide
SVDYNEPIIIKEN

SEQ ID NO: 81 - protein, 13 aa, Artificial
  Description of Artificial Sequence Synthetic peptide
CIEGDAKILTDRG

SEQ ID NO: 82 - protein, 13 aa, Artificial
  Description of Artificial Sequence Synthetic peptide
SLGRDELIFIKEG

SEQ ID NO: 83 - protein, 13 aa, Artificial
  Description of Artificial Sequence Synthetic peptide
SLPYDEKILIFEN

SEQ ID NO: 84 - protein, 13 aa, Artificial
  Description of Artificial Sequence Synthetic peptide
SLPYEEKIIIKEG

SEQ ID NO: 85 - protein, 13 aa, Artificial
  Description of Artificial Sequence Synthetic peptide
CVDGDTTVLLDGK

SEQ ID NO: 86 - protein, 13 aa, Artificial
  Description of Artificial Sequence Synthetic peptide
CFHPDEVLFIDRG

SEQ ID NO: 87 - protein, 13 aa, Artificial
  Description of Artificial Sequence Synthetic peptide
CLNANTEILQESG

SEQ ID NO: 88 - protein, 13 aa, Artificial
  Description of Artificial Sequence Synthetic peptide
CLHPDTYVILPDG

SEQ ID NO: 89 - protein, 13 aa, Artificial
  Description of Artificial Sequence Synthetic peptide
CLTPDTYVVLGDG

SEQ ID NO: 90 - protein, 13 aa, Artificial
  Description of Artificial Sequence Synthetic peptide
CLTGDTKVIVNGE

SEQ ID NO: 91 - protein, 13 aa, Artificial
  Description of Artificial Sequence Synthetic peptide
SVSKDTPILVKID

SEQ ID NO: 92 - protein, 13 aa, Artificial
  Description of Artificial Sequence Synthetic peptide
CLTGDAKITLPDE

SEQ ID NO: 93 - protein, 13 aa, Artificial
  Description of Artificial Sequence Synthetic peptide
MLSENTTILMANG

SEQ ID NO: 94 - protein, 14 aa, Artificial
  Description of Artificial Sequence Synthetic peptide
LLKFTCNATHELVV

SEQ ID NO: 95 - protein, 14 aa, Artificial
  Description of Artificial Sequence Synthetic peptide
LMDFTVSADHKLIL

SEQ ID NO: 96 - protein, 14 aa, Artificial
  Description of Artificial Sequence Synthetic peptide
GVDLFVTPNHRMYV

SEQ ID NO: 97 - protein, 14 aa, Artificial
  Description of Artificial Sequence Synthetic peptide
EKYLELTSNHKILT

SEQ ID NO: 98 - protein, 14 aa, Artificial
  Description of Artificial Sequence Synthetic peptide
GRTIKATANHRFLT

SEQ ID NO: 99 - protein, 14 aa, Artificial
  Description of Artificial Sequence Synthetic peptide
GAIVWATPDHKVLT

SEQ ID NO: 100 - protein, 14 aa, Artificial
  Description of Artificial Sequence Synthetic peptide
KSQFAATPNHLIRT

SEQ ID NO: 101 - protein, 14 aa, Artificial
  Description of Artificial Sequence Synthetic peptide
GRALEATGNHQFLV

SEQ ID NO: 102 - protein, 14 aa, Artificial
  Description of Artificial Sequence Synthetic peptide
GYEITGTSNHPLLC

SEQ ID NO: 103 - protein, 14 aa, Artificial
  Description of Artificial Sequence Synthetic peptide
GYEVTGTANHPLLC

SEQ ID NO: 104 - protein, 14 aa, Artificial
  Description of Artificial Sequence Synthetic peptide
GYEVTGTANHPLLC

SEQ ID NO: 105 - protein, 14 aa, Artificial
  Description of Artificial Sequence Synthetic peptide
GYEVTGTSNHPVLC

SEQ ID NO: 106 - protein, 14 aa, Artificial
  Description of Artificial Sequence Synthetic peptide
GLRVTGTANHPLLC

SEQ ID NO: 107 - protein, 14 aa, Artificial
  Description of Artificial Sequence Synthetic peptide
GRKINITAGHSLFT

SEQ ID NO: 108 - protein, 14 aa, Artificial
  Description of Artificial Sequence Synthetic peptide
GRKITITEGHSLFV

SEQ ID NO: 109 - protein, 14 aa, Artificial
  Description of Artificial Sequence Synthetic peptide
GRRIKITSGHSLFS

SEQ ID NO: 110 - protein, 14 aa, Artificial
  Description of Artificial Sequence Synthetic peptide
GRTIKVTRGHSLFK

SEQ ID NO: 111 - protein, 14 aa, Artificial
  Description of Artificial Sequence Synthetic peptide
SWYIDVTEDHSLIG

SEQ ID NO: 112 - protein, 14 aa, Artificial
  Description of Artificial Sequence Synthetic peptide
INGLKCTPNHKLPV

SEQ ID NO: 113 - protein, 14 aa, Artificial
  Description of Artificial Sequence Synthetic peptide
VNGLKCTPNHKIPL

SEQ ID NO: 114 - protein, 14 aa, Artificial
  Description of Artificial Sequence Synthetic peptide
PEEIILTPEHPVYA

SEQ ID NO: 115 - protein, 14 aa, Artificial
  Description of Artificial Sequence Synthetic peptide
GRVLEGSKDHPVLT

SEQ ID NO: 116 - protein, 14 aa, Artificial
  Description of Artificial Sequence Synthetic peptide
WHSITTTPEHPFLT

SEQ ID NO: 117 - protein, 14 aa, Artificial
  Description of Artificial Sequence Synthetic peptide
NKKVRVTRSHSVFT

SEQ ID NO: 118 - protein, 14 aa, Artificial
  Description of Artificial Sequence Synthetic peptide
KDTIKITPDHKFPV

SEQ ID NO: 119 - protein, 14 aa, Artificial
  Description of Artificial Sequence Synthetic peptide
GTSIIVTEDHSLFN

SEQ ID NO: 120 - protein, 14 aa, Artificial
  Description of Artificial Sequence Synthetic peptide
GKKVRVTGDHSVFT

SEQ ID NO: 121 - protein, 14 aa, Artificial
  Description of Artificial Sequence Synthetic peptide
GREITATPYHSFVI

SEQ ID NO: 122 - protein, 14 aa, Artificial
  Description of Artificial Sequence Synthetic peptide
GREIIATEDHPFYT

SEQ ID NO: 123 - protein, 14 aa, Artificial
  Description of Artificial Sequence Synthetic peptide
GREIKITKDHPVVI

SEQ ID NO: 124 - protein, 14 aa, Artificial
  Description of Artificial Sequence Synthetic peptide
GLEITTTPNHIFLV

SEQ ID NO: 125 - protein, 14 aa, Artificial
  Description of Artificial Sequence Synthetic peptide
FSELITTGEHKLFV

SEQ ID NO: 126 - protein, 14 aa, Artificial
  Description of Artificial Sequence Synthetic peptide
NYELKATPDHCLLV

SEQ ID NO: 127 - protein, 14 aa, Artificial
  Description of Artificial Sequence Synthetic peptide
GRELKVTTYHPLLI

SEQ ID NO: 128 - protein, 14 aa, Artificial
  Description of Artificial Sequence Synthetic peptide
GGYIELTGNHSIMM

SEQ ID NO: 129 - protein, 14 aa, Artificial
  Description of Artificial Sequence Synthetic peptide
GREIEATGDHKFLT

SEQ ID NO: 130 - protein, 14 aa, Artificial
  Description of Artificial Sequence Synthetic peptide
RLALQCTAGHKLSV

SEQ ID NO: 131 - protein, 9 aa, Artificial
  Description of Artificial Sequence Synthetic peptide
LLGLWIGDG

SEQ ID NO: 132 - protein, 9 aa, Artificial
  Description of Artificial Sequence Synthetic peptide
LLGTWAGIG

SEQ ID NO: 133 - protein, 9 aa, Artificial
  Description of Artificial Sequence Synthetic peptide
FFGLWIANG

SEQ ID NO: 134 - protein, 9 aa, Artificial
  Description of Artificial Sequence Synthetic peptide
LLGHLIGDG

SEQ ID NO: 135 - protein, 9 aa, Artificial
  Description of Artificial Sequence Synthetic peptide
LLGYLIGDG

SEQ ID NO: 136 - protein, 9 aa, Artificial
  Description of Artificial Sequence Synthetic peptide
VLGSLMGDG

SEQ ID NO: 137 - protein, 9 aa, Artificial
  Description of Artificial Sequence Synthetic peptide
LLGLWLGDG

SEQ ID NO: 138 - protein, 9 aa, Artificial
  Description of Artificial Sequence Synthetic peptide
LFGAFISGG

SEQ ID NO: 139 - protein, 9 aa, Artificial
  Description of Artificial Sequence Synthetic peptide
LLGAFISEG

SEQ ID NO: 140 - protein, 9 aa, Artificial
  Description of Artificial Sequence Synthetic peptide
HLGAFISEG

SEQ ID NO: 141 - protein, 9 aa, Artificial
  Description of Artificial Sequence Synthetic peptide
LLGAFISEG

SEQ ID NO: 142 - protein, 9 aa, Artificial
  Description of Artificial Sequence Synthetic peptide
LLGYYVSEG

SEQ ID NO: 143 - protein, 9 aa, Artificial
  Description of Artificial Sequence Synthetic peptide
LLGYYVSEG

SEQ ID NO: 144 - protein, 9 aa, Artificial
  Description of Artificial Sequence Synthetic peptide
LLGYYVSEG

SEQ ID NO: 145 - protein, 9 aa, Artificial
  Description of Artificial Sequence Synthetic peptide
FLGFFVTRG

SEQ ID NO: 146 - protein, 9 aa, Artificial
  Description of Artificial Sequence Synthetic peptide
LVGLIVGDG

SEQ ID NO: 147 - protein, 9 aa, Artificial
  Description of Artificial Sequence Synthetic peptide
LAGILLAEG

SEQ ID NO: 148 - protein, 9 aa, Artificial
  Description of Artificial Sequence Synthetic peptide
LIGILLAEG

SEQ ID NO: 149 - protein, 9 aa, Artificial
  Description of Artificial Sequence Synthetic peptide
LLGFAFGDG

SEQ ID NO: 150 - protein, 9 aa, Artificial
  Description of Artificial Sequence Synthetic peptide
FAGVMFGDG

SEQ ID NO: 151 - protein, 9 aa, Artificial
  Description of Artificial Sequence Synthetic peptide
ILGYIIAEG

SEQ ID NO: 152 - protein, 9 aa, Artificial
  Description of Artificial Sequence Synthetic peptide
LGGAVLSDG

SEQ ID NO: 153 - protein, 9 aa, Artificial
  Description of Artificial Sequence Synthetic peptide
FLGLFVAEG

SEQ ID NO: 154 - protein, 9 aa, Artificial
  Description of Artificial Sequence Synthetic peptide
LIGAFLSEG

SEQ ID NO: 155 - protein, 9 aa, Artificial
  Description of Artificial Sequence Synthetic peptide
FIGIYLAEG

SEQ ID NO: 156 - protein, 9 aa, Artificial
  Description of Artificial Sequence Synthetic peptide
LIGYYLSEG

SEQ ID NO: 157 - protein, 9 aa, Artificial
  Description of Artificial Sequence Synthetic peptide
FIGYFIGDG

SEQ ID NO: 158 - protein, 9 aa, Artificial
  Description of Artificial Sequence Synthetic peptide
IIGYIIGDG

SEQ ID NO: 159 - protein, 9 aa, Artificial
  Description of Artificial Sequence Synthetic peptide
FAGLVLGDG

SEQ ID NO: 160 - protein, 9 aa, Artificial
  Description of Artificial Sequence Synthetic peptide
WLGYFIGDG

SEQ ID NO: 161 - protein, 9 aa, Artificial
  Description of Artificial Sequence Synthetic peptide
MLGLYVAEG

SEQ ID NO: 162 - protein, 9 aa, Artificial
  Description of Artificial Sequence Synthetic peptide
LLGFIIGDG

SEQ ID NO: 163 - protein, 9 aa, Artificial
  Description of Artificial Sequence Synthetic peptide
MLGLWLGDG

SEQ ID NO: 164 - protein, 8 aa, Artificial
  Description of Artificial Sequence Synthetic peptide
VKNIPSFL

SEQ ID NO: 165 - protein, 8 aa, Artificial
  Description of Artificial Sequence Synthetic peptide
VKSIPQHI

SEQ ID NO: 166 - protein, 8 aa, Artificial
  Description of Artificial Sequence Synthetic peptide
NKYLPDWV

SEQ ID NO: 167 - protein, 8 aa, Artificial
  Description of Artificial Sequence Synthetic peptide
EKFVPNQV

SEQ ID NO: 168 - protein, 8 aa, Artificial
  Description of Artificial Sequence Synthetic peptide
EKTIPNWF

SEQ ID NO: 169 - protein, 8 aa, Artificial
  Description of Artificial Sequence Synthetic peptide
TKRLPAWI

SEQ ID NO: 170 - protein, 8 aa, Artificial
  Description of Artificial Sequence Synthetic peptide
DKLVPDWL

SEQ ID NO: 171 - protein, 8 aa, Artificial
  Description of Artificial Sequence Synthetic peptide
DTYVPEWM

SEQ ID NO: 172 - protein, 8 aa, Artificial
  Description of Artificial Sequence Synthetic peptide
DKAVPEWL

SEQ ID NO: 173 - protein, 8 aa, Artificial
  Description of Artificial Sequence Synthetic peptide
DKSVPEWL

SEQ ID NO: 174 - protein, 8 aa, Artificial
  Description of Artificial Sequence Synthetic peptide
NKRIPSVI

SEQ ID NO: 175 - protein, 8 aa, Artificial
  Description of Artificial Sequence Synthetic peptide
NKRVPEVI

SEQ ID NO: 176 - protein, 8 aa, Artificial
  Description of Artificial Sequence Synthetic peptide
NKRIPEFV

SEQ ID NO: 177 - protein, 8 aa, Artificial
  Description of Artificial Sequence Synthetic peptide
KKHIPEEL

SEQ ID NO: 178 - protein, 8 aa, Artificial
  Description of Artificial Sequence Synthetic peptide
RRKIPEFM

SEQ ID NO: 179 - protein, 8 aa, Artificial
  Description of Artificial Sequence Synthetic peptide
VKEIMDNI

SEQ ID NO: 180 - protein, 8 aa, Artificial
  Description of Artificial Sequence Synthetic peptide
RSRIPEKI

SEQ ID NO: 181 - protein, 8 aa, Artificial
  Description of Artificial Sequence Synthetic peptide
IYKIPEWI

SEQ ID NO: 182 - protein, 8 aa, Artificial
  Description of Artificial Sequence Synthetic peptide
NIKIPQIL

SEQ ID NO: 183 - protein, 8 aa, Artificial
  Description of Artificial Sequence Synthetic peptide
NKRIPSII

SEQ ID NO: 184 - protein, 8 aa, Artificial
  Description of Artificial Sequence Synthetic peptide
SRKIPSEI

SEQ ID NO: 185 - protein, 8 aa, Artificial
  Description of Artificial Sequence Synthetic peptide
NKNSPEFI

SEQ ID NO: 186 - protein, 8 aa, Artificial
  Description of Artificial Sequence Synthetic peptide
NKEIPSIL

SEQ ID NO: 187 - protein, 8 aa, Artificial
  Description of Artificial Sequence Synthetic peptide
TKKIAEFV

SEQ ID NO: 188 - protein, 8 aa, Artificial
  Description of Artificial Sequence Synthetic peptide
RKLIPLTY

SEQ ID NO: 189 - protein, 8 aa, Artificial
  Description of Artificial Sequence Synthetic peptide
NKNIPPQM

SEQ ID NO: 190 - protein, 8 aa, Artificial
  Description of Artificial Sequence Synthetic peptide
NKNIDAFC

SEQ ID NO: 191 - protein, 8 aa, Artificial
  Description of Artificial Sequence Synthetic peptide
NERTPEFV

SEQ ID NO: 192 - protein, 8 aa, Artificial
  Description of Artificial Sequence Synthetic peptide
IFSLPESY

SEQ ID NO: 193 - protein, 8 aa, Artificial
  Description of Artificial Sequence Synthetic peptide
KVRIPKEI

SEQ ID NO: 194 - protein, 8 aa, Artificial
  Description of Artificial Sequence Synthetic peptide
NKRIPDII

SEQ ID NO: 195 - protein, 8 aa, Artificial
  Description of Artificial Sequence Synthetic peptide
GYNIPKWI

SEQ ID NO: 196 - protein, 8 aa, Artificial
  Description of Artificial Sequence Synthetic peptide
EKQIPEFM

SEQ ID NO: 197 - protein, 10 aa, Artificial
  Description of Artificial Sequence Synthetic peptide
FLAGLIDSDG

SEQ ID NO: 198 - protein, 10 aa, Artificial
  Description of Artificial Sequence Synthetic peptide
LIAGLVDAAG

SEQ ID NO: 199 - protein, 10 aa, Artificial
  Description of Artificial Sequence Synthetic peptide
FLRHLWSTDG

SEQ ID NO: 200 - protein, 10 aa, Artificial
  Description of Artificial Sequence Synthetic peptide
LLFGLFESDG

SEQ ID NO: 201 - protein, 10 aa, Artificial
  Description of Artificial Sequence Synthetic peptide
LQRAVYLGDG

SEQ ID NO: 202 - protein, 10 aa, Artificial
  Description of Artificial Sequence Synthetic peptide
LIGGLVDADG

SEQ ID NO: 203 - protein, 10 aa, Artificial
  Description of Artificial Sequence Synthetic peptide
FLQALFEGEG

SEQ ID NO: 204 - protein, 10 aa, Artificial
  Description of Artificial Sequence Synthetic peptide
FLQALFEGDG

SEQ ID NO: 205 - protein, 10 aa, Artificial
  Description of Artificial Sequence Synthetic peptide
FLQALFEGDG

SEQ ID NO: 206 - protein, 10 aa, Artificial
  Description of Artificial Sequence Synthetic peptide
FLQALFEGDG

SEQ ID NO: 207 - protein, 10 aa, Artificial
  Description of Artificial Sequence Synthetic peptide
FLEAYFTGDG

SEQ ID NO: 208 - protein, 10 aa, Artificial
  Description of Artificial Sequence Synthetic peptide
FLEGYFIGDG

SEQ ID NO: 209 - protein, 10 aa, Artificial
  Description of Artificial Sequence Synthetic peptide
FLEGYFIGDG

SEQ ID NO: 210 - protein, 10 aa, Artificial
  Description of Artificial Sequence Synthetic peptide
FLRGLFSADG

SEQ ID NO: 211 - protein, 10 aa, Artificial
  Description of Artificial Sequence Synthetic peptide
VLRGFFEGDG

SEQ ID NO: 212 - protein, 10 aa, Artificial
  Description of Artificial Sequence Synthetic peptide
ILRGFFEGDG

SEQ ID NO: 213 - protein, 10 aa, Artificial
  Description of Artificial Sequence Synthetic peptide
RLVGYFLSEG

SEQ ID NO: 214 - protein, 10 aa, Artificial
  Description of Artificial Sequence Synthetic peptide
FLAGLFGADG

SEQ ID NO: 215 - protein, 10 aa, Artificial
  Description of Artificial Sequence Synthetic peptide
FIKGYFDADG

SEQ ID NO: 216 - protein, 10 aa, Artificial
  Description of Artificial Sequence Synthetic peptide
FIDGIFNGKD

SEQ ID NO: 217 - protein, 10 aa, Artificial
  Description of Artificial Sequence Synthetic peptide
LIAGFVDGDG

SEQ ID NO: 218 - protein, 10 aa, Artificial
  Description of Artificial Sequence Synthetic peptide
FLGGLISGDG

SEQ ID NO: 219 - protein, 10 aa, Artificial
  Description of Artificial Sequence Synthetic peptide
LIKGYIDGDG

SEQ ID NO: 220 - protein, 10 aa, Artificial
  Description of Artificial Sequence Synthetic peptide
LIRGYFDGDG

SEQ ID NO: 221 - protein, 10 aa, Artificial
  Description of Artificial Sequence Synthetic peptide
RIVGHVMGDG

SEQ ID NO: 222 - protein, 10 aa, Artificial
  Description of Artificial Sequence Synthetic peptide
FLKGLFRGDG

SEQ ID NO: 223 - protein, 10 aa, Artificial
  Description of Artificial Sequence Synthetic peptide
LIAGLFDSDG

SEQ ID NO: 224 - protein, 10 aa, Artificial
  Description of Artificial Sequence Synthetic peptide
YLRGIFDAEG

SEQ ID NO: 225 - protein, 10 aa, Artificial
  Description of Artificial Sequence Synthetic peptide
LIAGYFDTDG

SEQ ID NO: 226 - protein, 10 aa, Artificial
  Description of Artificial Sequence Synthetic peptide
FLRAYFDCDG

SEQ ID NO: 227 - protein, 10 aa, Artificial
  Description of Artificial Sequence Synthetic peptide
FLKGLADGDS

SEQ ID NO: 228 - protein, 10 aa, Artificial
  Description of Artificial Sequence Synthetic peptide
FLRGLFGADG

SEQ ID NO: 229 - protein, 10 aa, Artificial
  Description of Artificial Sequence Synthetic peptide
FLAGLIDSDG

SEQ ID NO: 230 - protein, 19 aa, Artificial
  Description of Artificial Sequence Synthetic peptide
TIHTSVRDGLVSLARSLGL

SEQ ID NO: 231 - protein, 19 aa, Artificial
  Description of Artificial Sequence Synthetic peptide
TSFRHVARGLVKIAHSLGI

SEQ ID NO: 232 - protein, 19 aa, Artificial
  Description of Artificial Sequence Synthetic peptide
STSERFANDVSRLALHAGT

SEQ ID NO: 233 - protein, 19 aa, Artificial
  Description of Artificial Sequence Synthetic peptide
TSSEKLAKDVQSLLLKLGI

SEQ ID NO: 234 - protein, 19 aa, Artificial
  Description of Artificial Sequence Synthetic peptide
TTSEQLAHQIHWLLLRYGV

SEQ ID NO: 235 - protein, 19 aa, Artificial
  Description of Artificial Sequence Synthetic peptide
FSLEEYLKALTPLVLAIWY

SEQ ID NO: 236 - protein, 19 aa, Artificial
  Description of Artificial Sequence Synthetic peptide
FASRELLEDVRQLAIGCGL

SEQ ID NO: 237 - protein, 19 aa, Artificial
  Description of Artificial Sequence Synthetic peptide
TLSERLAADVQQMLLEFGV

SEQ ID NO: 238 - protein, 19 aa, Artificial
  Description of Artificial Sequence Synthetic peptide
TVSKQLAMDVQQMLLEFGV

SEQ ID NO: 239 - protein, 19 aa, Artificial
  Description of Artificial Sequence Synthetic peptide
TRSGRLAKDIQQMLLEFGV

SEQ ID NO: 240 - protein, 19 aa, Artificial
  Description of Artificial Sequence Synthetic peptide
TRSRQLAIDVQQMLLEFGV

SEQ ID NO: 241 - protein, 19 aa, Artificial
  Description of Artificial Sequence Synthetic peptide
TKSELLANQLVFLLNSLGI

SEQ ID NO: 242 - protein, 19 aa, Artificial
  Description of Artificial Sequence Synthetic peptide
TKSELLVNGLVLLLNSLGV

SEQ ID NO: 243 - protein, 19 aa, Artificial
  Description of Artificial Sequence Synthetic peptide
NEKRALANQLVLLLNSVGV

SEQ ID NO: 244 - protein, 19 aa, Artificial
  Description of Artificial Sequence Synthetic peptide
AKDEKYLNQLMILFNLVGI

SEQ ID NO: 245 - protein, 19 aa, Artificial
  Description of Artificial Sequence Synthetic peptide
NIDADFLREVRKLLWIVGI

SEQ ID NO: 246 - protein, 19 aa, Artificial
  Description of Artificial Sequence Synthetic peptide
TKNEWKIKLVSKLLSQLGI

SEQ ID NO: 247 - protein, 19 aa, Artificial
  Description of Artificial Sequence Synthetic peptide
TNNYDKIKFIASLLDRLGI

SEQ ID NO: 248 - protein, 19 aa, Artificial
  Description of Artificial Sequence Synthetic peptide
TTSEILMNQLRLISLRLGF

SEQ ID NO: 249 - protein, 19 aa, Artificial
  Description of Artificial Sequence Synthetic peptide
ENILEFLNEIKLLLAEFDI

SEQ ID NO: 250 - protein, 19 aa, Artificial
  Description of Artificial Sequence Synthetic peptide
SASKEFIEGLSILLLRFEI

SEQ ID NO: 251 - protein, 19 aa, Artificial
  Description of Artificial Sequence Synthetic peptide
FVSKELAEDVIFLLLQIKE

SEQ ID NO: 252 - protein, 19 aa, Artificial
  Description of Artificial Sequence Synthetic peptide
SSHIKKIEGLIVGLYRLGI

SEQ ID NO: 253 - protein, 19 aa, Artificial
  Description of Artificial Sequence Synthetic peptide
TTSEQLLGQLHLLLSDLGM

SEQ ID NO: 254 - protein, 19 aa, Artificial
  Description of Artificial Sequence Synthetic peptide
TTSETLRDTLCLALKILGI

SEQ ID NO: 255 - protein, 19 aa, Artificial
  Description of Artificial Sequence Synthetic peptide
SNSKELIDGIAILLARFNI

SEQ ID NO: 256 - protein, 19 aa, Artificial
  Description of Artificial Sequence Synthetic peptide
YSFEVRKKSLCILLKALGC

SEQ ID NO: 257 - protein, 19 aa, Artificial
  Description of Artificial Sequence Synthetic peptide
TVSKKMAESLLILLQLLGI

SEQ ID NO: 258 - protein, 19 aa, Artificial
  Description of Artificial Sequence Synthetic peptide
SISEKLVEQLQFVLLRFGI

SEQ ID NO: 259 - protein, 19 aa, Artificial
  Description of Artificial Sequence Synthetic peptide
MTSKCFIKEIQFLLLRFGI

SEQ ID NO: 260 - protein, 19 aa, Artificial
  Description of Artificial Sequence Synthetic peptide
SKRRDVLEKIGIYLNSIGI

SEQ ID NO: 261 - protein, 19 aa, Artificial
  Description of Artificial Sequence Synthetic peptide
TASKEMAEDLVYALLRFGI

SEQ ID NO: 262 - protein, 19 aa, Artificial
  Description of Artificial Sequence Synthetic peptide
SKSDNLLIDTVWLARISGI

SEQ ID NO: 263 - protein, 19 aa, Artificial
  Description of Artificial Sequence Synthetic peptide
DKTLEFFEEVKKMLEEFEV

SEQ ID NO: 264 - protein, 19 aa, Artificial
  Description of Artificial Sequence Synthetic peptide
TVYSSIMDGIVHISRSLGM

SEQ ID NO: 265 - protein, 14 aa, Artificial
  Description of Artificial Sequence Synthetic peptide
YGITLSDDSDHQFL

SEQ ID NO: 266 - protein, 14 aa, Artificial
  Description of Artificial Sequence Synthetic peptide
YGITLAEETDHQFL

SEQ ID NO: 267 - protein, 14 aa, Artificial
  Description of Artificial Sequence Synthetic peptide
PVYCLTGPNNVFYV

SEQ ID NO: 268 - protein, 14 aa, Artificial
  Description of Artificial Sequence Synthetic peptide
NVFDFAANPIPNFI

SEQ ID NO: 269 - protein, 14 aa, Artificial
  Description of Artificial Sequence Synthetic peptide
EVFDLTVPGPHNFV

SEQ ID NO: 270 - protein, 14 aa, Artificial
  Description of Artificial Sequence Synthetic peptide
RTFDLEVEELHTLV

SEQ ID NO: 271 - protein, 14 aa, Artificial
  Description of Artificial Sequence Synthetic peptide
NRFDIEVEGNHNYF

SEQ ID NO: 272 - protein, 14 aa, Artificial
  Description of Artificial Sequence Synthetic peptide
PTYDIQVVGLENFV

SEQ ID NO: 273 - protein, 14 aa, Artificial
  Description of Artificial Sequence Synthetic peptide
PVFSLHVDTEDHSF

SEQ ID NO: 274 - protein, 14 aa, Artificial
  Description of Artificial Sequence Synthetic peptide
PVYSLRVDTDDHAF

SEQ ID NO: 275 - protein, 14 aa, Artificial
  Description of Artificial Sequence Synthetic peptide
PVYSLRVDTDDHSF

SEQ ID NO: 276 - protein, 14 aa, Artificial
  Description of Artificial Sequence Synthetic peptide
PVYSLRVDSEDHAF

SEQ ID NO: 277 - protein, 14 aa, Artificial
  Description of Artificial Sequence Synthetic peptide
PVYSLRVDTADHAF

SEQ ID NO: 278 - protein, 14 aa, Artificial
  Description of Artificial Sequence Synthetic peptide
YVYDLSVEDNENFL

SEQ ID NO: 279 - protein, 14 aa, Artificial
  Description of Artificial Sequence Synthetic peptide
YVYDLSVDEDENFL

SEQ ID NO: 280 - protein, 14 aa, Artificial
  Description of Artificial Sequence Synthetic peptide
YVYDLSVEDNENFL

SEQ ID NO: 281 - protein, 14 aa, Artificial
  Description of Artificial Sequence Synthetic peptide
YVYDLSVEDNENFL

SEQ ID NO: 282 - protein, 14 aa, Artificial
  Description of Artificial Sequence Synthetic peptide
YVYDIEVEETHRFF

SEQ ID NO: 283 - protein, 14 aa, Artificial
  Description of Artificial Sequence Synthetic peptide
KVYDLTLEGTPYYF

SEQ ID NO: 284 - protein, 14 aa, Artificial
  Description of Artificial Sequence Synthetic peptide
EVYDLTLEGRPYYF

SEQ ID NO: 285 - protein, 14 aa, Artificial
  Description of Artificial Sequence Synthetic peptide
FVYNLEVEDDSSYV

SEQ ID NO: 286 - protein, 14 aa, Artificial
  Description of Artificial Sequence Synthetic peptide
KLYDVGIVSKEHNF

SEQ ID NO: 287 - protein, 14 aa, Artificial
  Description of Artificial Sequence Synthetic peptide
YVYDLTTETHNFIA

SEQ ID NO: 288 - protein, 14 aa, Artificial
  Description of Artificial Sequence Synthetic peptide
YAYDLTVPNAENFV

SEQ ID NO: 289 - protein, 14 aa, Artificial
  Description of Artificial Sequence Synthetic peptide
EVYNITVKAENEFN

SEQ ID NO: 290 - protein, 14 aa, Artificial
  Description of Artificial Sequence Synthetic peptide
YVYDLSVEDNENFI

SEQ ID NO: 291 - protein, 14 aa, Artificial
  Description of Artificial Sequence Synthetic peptide
YVYDISVEGTENFI

SEQ ID NO: 292 - protein, 14 aa, Artificial
  Description of Artificial Sequence Synthetic peptide
YVYDISVEGLETFT

SEQ ID NO: 293 - protein, 14 aa, Artificial
  Description of Artificial Sequence Synthetic peptide
YVYDITTISETHNF

SEQ ID NO: 294 - protein, 14 aa, Artificial
  Description of Artificial Sequence Synthetic peptide
YVYSVETENSLLIT

SEQ ID NO: 295 - protein, 14 aa, Artificial
  Description of Artificial Sequence Synthetic peptide
YAYDIELPDDGSNS

SEQ ID NO: 296 - protein, 14 aa, Artificial
  Description of Artificial Sequence Synthetic peptide
YVYDLEVEDYHNFI

SEQ ID NO: 297 - protein, 14 aa, Artificial
  Description of Artificial Sequence Synthetic peptide
YVYDLSIKHNQNFI

SEQ ID NO: 298 - protein, 14 aa, Artificial
  Description of Artificial Sequence Synthetic peptide
TIYDLHVPRYHNFI

SEQ ID NO: 299 - protein, 14 aa, Artificial
  Description of Artificial Sequence Synthetic peptide
FVYDVSVPNNEMFF

SEQ ID NO: 300 - protein, 14 aa, Artificial
  Description of Artificial Sequence Synthetic peptide
DVYDITCHKDPSFI

SEQ ID NO: 301 - protein, 14 aa, Artificial
  Description of Artificial Sequence Synthetic peptide
TVYGLTIEGHKNFL

SEQ ID NO: 302 - protein, 8 aa, Artificial
  Description of Artificial Sequence Synthetic peptide
NQVVVHNC

SEQ ID NO: 303 - protein, 8 aa, Artificial
  Description of Artificial Sequence Synthetic peptide
NMALVHNC

SEQ ID NO: 304 - protein, 8 aa, Artificial
  Description of Artificial Sequence Synthetic peptide
KAVWTGNS

SEQ ID NO: 305 - protein, 8 aa, Artificial
  Description of Artificial Sequence Synthetic peptide
NNIIVHNS

SEQ ID NO: 306 - protein, 8 aa, Artificial
  Description of Artificial Sequence Synthetic peptide
NDIIVHNS

SEQ ID NO: 307 - protein, 8 aa, Artificial
  Description of Artificial Sequence Synthetic peptide
EGVVVHNC

SEQ ID NO: 308 - protein, 8 aa, Artificial
  Description of Artificial Sequence Synthetic peptide
DGVMVHNS

SEQ ID NO: 309 - protein, 8 aa, Artificial
  Description of Artificial Sequence Synthetic peptide
NGIVAHNS

SEQ ID NO: 310 - protein, 8 aa, Artificial
  Description of Artificial Sequence Synthetic peptide
NGFISHNT

SEQ ID NO: 311 - protein, 8 aa, Artificial
  Description of Artificial Sequence Synthetic peptide
NGFVSHNT

SEQ ID NO: 312 - protein, 8 aa, Artificial
  Description of Artificial Sequence Synthetic peptide
NGFVSHNT

SEQ ID NO: 313 - protein, 8 aa, Artificial
  Description of Artificial Sequence Synthetic peptide
NGFVSHNT

SEQ ID NO: 314 - protein, 8 aa, Artificial
  Description of Artificial Sequence Synthetic peptide
NGFVSHNT

SEQ ID NO: 315 - protein, 8 aa, Artificial
  Description of Artificial Sequence Synthetic peptide
GLLYAHNS

SEQ ID NO: 316 - protein, 8 aa, Artificial
  Description of Artificial Sequence Synthetic peptide
GFLYAHNS

SEQ ID NO: 317 - protein, 8 aa, Artificial
  Description of Artificial Sequence Synthetic peptide
GLVYAHNS

SEQ ID NO: 318 - protein, 8 aa, Artificial
  Description of Artificial Sequence Synthetic peptide
NNIYAHNS

SEQ ID NO: 319 - protein, 8 aa, Artificial
  Description of Artificial Sequence Synthetic peptide
NNILVHNT

SEQ ID NO: 320 - protein, 8 aa, Artificial
  Description of Artificial Sequence Synthetic peptide
NGILTHNS

SEQ ID NO: 321 - protein, 8 aa, Artificial
  Description of Artificial Sequence Synthetic peptide
NGILTHNS

SEQ ID NO: 322 - protein, 8 aa, Artificial
  Description of Artificial Sequence Synthetic peptide
VSGTLHNC

SEQ ID NO: 323 - protein, 8 aa, Artificial
  Description of Artificial Sequence Synthetic peptide
NSIVVHNC

SEQ ID NO: 324 - protein, 8 aa, Artificial
  Description of Artificial Sequence Synthetic peptide
NGIVVHNT

SEQ ID NO: 325 - protein, 8 aa, Artificial
  Description of Artificial Sequence Synthetic peptide
GGFVLHNT

SEQ ID NO: 326 - protein, 8 aa, Artificial
  Description of Artificial Sequence Synthetic peptide
TPIVVFNC

SEQ ID NO: 327 - protein, 8 aa, Artificial
  Description of Artificial Sequence Synthetic peptide
TGILCHNT

SEQ ID NO: 328 - protein, 8 aa, Artificial
  Description of Artificial Sequence Synthetic peptide
GFICLHNT

SEQ ID NO: 329 - protein, 8 aa, Artificial
  Description of Artificial Sequence Synthetic peptide
DGVLTHNT

SEQ ID NO: 330 - protein, 8 aa, Artificial
  Description of Artificial Sequence Synthetic peptide
NGFLTGNC

SEQ ID NO: 331 - protein, 8 aa, Artificial
  Description of Artificial Sequence Synthetic peptide
YGILIHNC

SEQ ID NO: 332 - protein, 8 aa, Artificial
  Description of Artificial Sequence Synthetic peptide
NGFIVHNS

SEQ ID NO: 333 - protein, 8 aa, Artificial
  Description of Artificial Sequence Synthetic peptide
NLIINHNS

SEQ ID NO: 334 - protein, 8 aa, Artificial
  Description of Artificial Sequence Synthetic peptide
NGVISHNC

SEQ ID NO: 335 - protein, 8 aa, Artificial
  Description of Artificial Sequence Synthetic peptide
LPTILHNT

SEQ ID NO: 336 - protein, 8 aa, Artificial
  Description of Artificial Sequence Synthetic peptide
VPILLHNS

SEQ ID NO: 337 - protein, 8 aa, Artificial
  Description of Artificial Sequence Synthetic peptide
NGFVSHNC

SEQ ID NO: 338 - protein, 5 aa, Artificial Sequence
  Description of Artificial Sequence Synthetic peptide
SEEDL

SEQ ID NO: 339 - protein, 5 aa, Artificial Sequence
  Description of Artificial Sequence Synthetic peptide
EQKLI

SEQ ID NO: 340 - protein, 5 aa, Artificial Sequence
  Description of Artificial Sequence Synthetic peptide
CGPGR

SEQ ID NO: 341 - protein, 10 aa, Artificial Sequence
  Description of Artificial Sequence Synthetic peptide
LFLYGPWKDL

SEQ ID NO: 342 - protein, 15 aa, Artificial Sequence
  Description of Artificial Sequence Synthetic peptide
CGRHYSCGSECCNPA

SEQ ID NO: 343 - protein, 15 aa, Artificial Sequence
  Description of Artificial Sequence Synthetic peptide
SCGSECCNPACGRHY

SEQ ID NO: 344 - protein, 15 aa, Artificial Sequence
  Description of Artificial Sequence Synthetic peptide
CCNPACGRHYSCGSE

SEQ ID NO: 345 - DNA, 29 bp, Homo sapiens
  CDS (3)..(29)
  modified_base (7)..(11): a, c, g, or t
ccatggnnnnnggctgcttcccgggatcc

SEQ ID NO: 346 - protein, 9 aa, Homo sapiens
  MOD_RES (2): Val, Ala, Asp, or Gly
MXXGCFPGS